(19) United States
(12) Patent Application Publication
(10) Pub. No.: US 2025/0009823 A1
(43) Pub. Date: Jan. 9, 2025

(54) ONCOLYTIC VIRUS REGIMENS FOR THE TREATMENT OF CANCER

(71) Applicant: Istari Oncology, Inc., Morrisville, NC (US)

(72) Inventors: Daniel Corum, Morrisville, NC (US); Shannon Morris, Morrisville, NC (US)

(73) Assignee: Istari Oncology, Inc., Morrisville, NC (US)

(21) Appl. No.: 18/778,670

(22) Filed: Jul. 19, 2024

Related U.S. Application Data
(63) Continuation of application No. PCT/US23/11176, filed on Jan. 19, 2023.
(60) Provisional application No. 63/414,808, filed on Oct. 10, 2022.
(60) Provisional application No. 63/341,330, filed on May 12, 2022.
(60) Provisional application No. 63/310,008, filed on Feb. 14, 2022.
(60) Provisional application No. 63/301,008, filed on Jan. 19, 2022.

(51) Int. Cl. A61K 35/768; A61K 39/395; A61P 35/00

(52) U.S. Cl. CPC A61K 35/768; A61K 39/3955; A61P 35/00

(57) ABSTRACT

The addition of lerapolturev in a very specific dosage regimen in combination with a checkpoint inhibitor provides superior results in the treatment of a cancer or a tumor. The unexpected discovery is that the specifically-timed administration of selectively high-dosed lerapolturev in combination with an immune checkpoint inhibitor has a profound effect on the immune cells in the cancer microenvironment.

### CROSS-REFERENCE TO RELATED APPLICATIONS

This application is a continuation of International Patent Application No. PCT/US2023/011176, filed Jan. 19, 2023, which claims the benefit of and priority to U.S. Provisional Application No. 63/301,008 filed Jan. 19, 2022, U.S. Provisional Application No. 63/310,008 filed Feb. 14, 2022, U.S. Provisional Application No. 63/341,330 filed May 12, 2022, and U.S. Provisional Application No. 63/414,808 filed Oct. 10, 2022. The entirety of each of these applications is hereby incorporated by reference herein for all purposes.

### INCORPORATION BY REFERENCE

The contents of the xml file named “21081-032WO1_ST26.xml” which was created on Jan. 2, 2023, and is 11.3 KB in size, are hereby incorporated by reference in their entirety.

### FIELD OF THE INVENTION

This invention relates to improved methods for treating cancer that alter the tumor microenvironment in a manner that promotes a pro-inflammatory microenvironment in multiple cancer types through the administration of a high dose of an oncolytic virus in combination or alteration with an immune checkpoint inhibitor (ICI).

### BACKGROUND OF THE INVENTION

Cancer is among the leading causes of death worldwide (WHO Global Health Estimates (2019)). In 2020, an estimated 19.2 million new cases of cancer were diagnosed and approximately 10 million people died from the disease (The Global Cancer Observatory (2020)). By 2040, the number of new cases and deaths are expected to nearly double (The Global Cancer Observatory (2018)).

A particularly devastating cancer is metastatic melanoma, often a fatal disease, with less than 28% of patients surviving 5 years (Cancer Statistics Review, 1975-2015). However, many patients are diagnosed with early-stage disease, where if well managed, the chance of cure can be quite high. Unfortunately, patients who recur or are diagnosed with advanced disease suffer a more aggressive disease course, making the likelihood of cure much less. For patients with Stage III disease, over 70% of patients' first recurrence was regional/nodal or systemic (Romano et al. J Clin Oncol Off J Am Soc Clin Oncol. 28(18):3042-7(2010)), meaning localized therapies (e.g., surgery or isolated limb perfusion) are likely to have limited effect. Therefore, systemic therapies are often indicated, to mitigate further systemic spread and extend life.

A major breakthrough in the field of cancer therapy in the past decade has been the introduction of T cell targeting immunomodulators which regulate the blockade of immune checkpoint molecules programmed cell death protein 1 (PD-1), programmed death-ligand-1 (PD-L1) and cytotoxic T-lymphocyte-associated protein 4 (CTLA-4). The PD-1, PD-L1, and CTLA-4 immune checkpoint molecules function as physiological brakes on unrestrained cytotoxic T cell effector function. PD-1 (CD279) is an inhibitor transmembrane protein expressed on T cells, B cells, Natural Killer cells (NKs), and Myeloid-Derived Suppressor Cells (MDSCs). PD-L1 (CD274) is expressed on the surface of multiple tissue types, including many tumor cells and hematopoietic cells. CTLA-4 (CD152) is a B7/Cd28 family member and mediates immunosuppression by indirectly diminishing signaling through the co-stimulatory receptor CD28. Immune checkpoint blockade has been found to unleash T cell activity to promote a systemic anti-tumor response by the host immune system, demonstrating remarkable success in clinical trials (Wolchok et al. N Engl J Med. 369:122-33(2013); Topalian et al. N Engl J Med. 366:2443-54(2012); Brahmer et al. N Engl J Med. 366:2455-2465(2012); Robert et al. Lancet. 384:1109-17(2014); Ribas et al. JAMA. 315:1600-09(2016); Powles et al. Nature. 515:558-62(2014); Brahmer et al. J Clin Oncol. 28:3167-75(2010); Topalian et al. J Clin Oncol. 32:1020-30(2014); Hamid et al. N Engl J Med. 369:134-44(2013); Garon et al. N Engl J Med. 372:2018-28(2015)).

A human IgG1 K anti-CTLA-4 monoclonal antibody used as an immune checkpoint inhibitor (ICI), ipilimumab (sold under the brand name YERVOY®), was approved by the United States (US) Food and Drug Administration (FDA) in 2011 for the treatment of unresectable or metastatic melanoma. Ipilimumab remains the only FDA-approved CTLA-4 inhibitor. Since the approval of ipilimumab by the FDA in 2011, six more monoclonal antibody ICIs have been approved for cancer therapy: PD-1 inhibitors nivolumab (OPDIVO®), pembrolizumab (KEYTRUDA®), cemiplimab (LIBTAYO®) and PD-L1 inhibitors atezolizumab (TECENTRIQ®), avelumab (BEVENCIO®), and durvalumab (IMFINZI®). Immunomodulatory ICIs have become some of the most widely prescribed anticancer therapies. International recognition of these breakthroughs in cancer therapy was recognized in 2018, when the researchers James Allison and Tasuku Honjo, credited with pioneering the use of ICIs to treat cancers, were awarded the Nobel Prize in Medicine (Ledford et al. Nature. 562(7725):20-21(2018)).

FDA approved ICIs which target PD-1/PD-L1/CTLA-4 pathways can be used alone or in combination in the front-line setting. When used as a monotherapy, fewer than 40% of patients respond to PD-1 inhibitor therapy, suggesting that greater than 60% of the general population has primary PD-1 resistance (Robert et al. N Engl J Med. 372(26):2521-32(2015); Robert et al. N Engl J Med. 372(4):320-30(2015)). For example, of melanoma patients who do respond to PD-1 inhibitor therapy, up to 25% of this subpopulation develop an acquired resistance (Ribas et al. JAMA 315(15):1600-09(2016)). Given this, researchers contemplated combining different ICIs together could overcome primary or acquired resistance to ICI therapy. For example, the combined blockade of PD-1 and CTLA-4 can increase the response rate to approximately 50%, demonstrating an OS advantage that led to FDA approval of the combination. Clinical trials demonstrate this regimen, however, is associated with significant toxicity as over half of all patients experienced Grade 3/4 adverse events (Aes) (Larkin et al. N Engl J Med. 373(1):23-34(2015)). Indeed, a significant proportion of melanoma patients do not respond to currently approved ICI immunotherapy combinations even when accepting the risk for significant toxicity. This suggests alternative therapies need to be identified that safely trigger and activate the immune system to generate an anti-tumor immune response.

A major recent development in this area has been the use of oncolytic viruses, which can provoke a systemic anti-tumor response, and depending on the virus, can directly target and kill tumor cells. One such oncolytic virus, talimogene laherparepvec (T-VEC, IMLYGIC®), a modified herpes simplex type 1 virus expressing human granulocyte macrophage colony-stimulating factor (GM-CSF), was approved by the FDA in 2015 to treat melanoma. IMLYGIC® treatment importantly demonstrated increased durable response rate (DRR, defined as the percent of patients with complete response (CR) or partial response (PR) maintained continuously for a minimum of 6 months) compared to patients administered GM-CSF alone. In the IMLYGIC® arm the DRR was 16.3%, whereas only 2.1% of patients in the GM-CSF arm demonstrated a DRR. However, no statistically significant difference in overall survival (OS) was observed between the IMLYGIC® and the GM-CSF arms. The median OS in the overall study population was 22.9 months in the IMLYGIC arm and 19.0 months in the GM-CSF arm (p=0.116) (Andtbacka et al. J Clin Oncol. 33(25):2780-8(2015)). Despite these results which demonstrate oncolytic viruses indeed affect cancer growth, oncolytic viral therapies have not shown comprehensive efficacy when used alone, and IMLYGIC® remains the only oncolytic virus approved by the FDA for the treatment of cancer.

Recently, trials were designed to add two divergent treatment strategies together: ICI immunotherapy and oncolytic viral therapy, in the hopes of enhancing the anti-tumor response compared to either agent alone. Data from a Phase 1b trial investigating IMLYGIC® in combination with PD-1 inhibitor pembrolizumab in advanced PD-1 naïve melanoma showed that greater than 60% of patients achieved an objective response (OR), with multiple responses ongoing at the time of trial study publication (median OS and progression-free survival not reached) (Ribas et al. Cell. 170(6):1109-19(2017)). Response to the combination therapy was associated with a comprehensive immune response, as increased CD8+ T-cell infiltration and increased expression of PD-L1 (the immunosuppressive ligand of PD-1) was observed in the tumor microenvironment (TME) following treatment. These observations suggest an oncolytic virus in combination with an PD-1 inhibitor could potentially overcome mechanisms of PD-1 inhibitor resistance. Despite these encouraging data, safety concerns remain a vital consideration, as treatment with the combination was associated with several side effects including fever, chills, fatigue and one case of cytokine release syndrome (CRS) resulting in hospitalization. These side effects are not among the AEs normally attributable to pembrolizumab monotherapy treatment. Aes associated with oncolytic viral therapy also are of concern, as herpetic infections have been reported in patients administered IMLYGIC®. Whether these results can be recapitulated in an PD-1 inhibitor resistant population is currently under investigation.

Recently, researchers have attempted to determine whether there is any potential for clinical benefit from anti-PD-1 re-challenge in patients who previously experienced clinical progression on PD-1/PD-L1 inhibitor therapy or from treating beyond progression (TBP) in patients that are currently failing an PD-1/PD-L1 inhibitor regimen. No study has formally addressed whether PD-1 inhibitor re-challenge following ICI discontinuation is beneficial (Saleh et al. Immunotherapy. 10(5):345-7(2018)), which suggests this approach may not viable.

An FDA meta-analysis of melanoma clinical trials that allowed continued PD-1 inhibitor therapy beyond RECIST-defined progression has shown that TBP with a PD-1 inhibitor can result in an ORR of up to 19% (Beaver et al. Lancet Oncol. 19(2):229-39(2018)). However, 66% of TBP patients in this analysis progressed at their first post-treatment visit, with a median TBP duration of 1.41 months (IQR: 0.69-4.86 months). Of these patients with RECIST-defined progressive disease (PD) at their first post-treatment scan, approximately 36% discontinued due to the appearance of a new lesion without target lesion progression. This result is indicative of an immunoprogressive phenomenon which may result in subsequent lesion contraction.

In addition, the protocols allowing TBP within this meta-analysis had specific criteria with which to identify the patients who were allowed to continue PD-1 inhibitor treatment. These criteria included: absence of clinical progression (including laboratory values), no decline in performance status, and absence of disease progression in areas requiring immediate medical treatment (Beaver et al. Lancet Oncol. 19(2):229-39(2018)). Given that the median time to response for PD-1 inhibitor treatment is between 2-3 months (Weber et al. Lancet Oncol. 16(4):375-84(2015); Hamid et al. Ann Oncol. 30(4):582-8(2019)), it is possible that TBP in this select, well-performing population extended the time for patients to achieve a treatment response. In contrast, nearly two-thirds of patients within the TBP cohort would have been excluded had the definition of PD-1/PD-L1 inhibitor resistance from the Society for Immunotherapy of Cancer (SITC) Immunotherapy Resistance Task force been applied to the population of this meta-analysis study (i.e., PD confirmation by repeat imaging at least 4 weeks later) (Kluger et al. J Immunother Cancer. 8(1):e000398(2020)). Thus, it can be estimated that a true ORR for PD-1 inhibitor TBP in patients per the SITC Immunotherapy Resistance Task Force definition is no greater than 6%. Therefore, ICI re-challenge therapy likely poses significant limitations as a long-term cancer strategy.

Alternatively, it was hypothesized that ICI therapy to multiple immune checkpoints may overcome the limitations of targeting a single checkpoint. For example, a recent retrospective analysis study investigated whether the CTLA-4 inhibitor ipilimumab in combination with PD-1 inhibitor treatment achieved improved outcomes in an PD-1 inhibitor refractory population relative to ipilimumab alone. The ORR in the combination arm was 31% vs 12% for ipilimumab alone, and OS at 1-year for the combination was also superior versus ipilimumab alone. In addition, the FDA approved the combination of PD-1 inhibitor nivolumab and LAG-3 inhibitor relatlimab (OPDUALAG™) for the treatment of unresectable or metastatic melanoma in March 2022, showing a slight benefit in progression-free survival in the frontline setting (Tawbi, H. A. et al. Relatlimab and Nivolumab versus Nivolumab in Untreated Advanced Melanoma. N Engl J Med. 386:24-34(2022); U.S. Food & Drug Administration. FDA approves Opdualag for unresectable or metastatic melanoma. (Mar. 21, 2022); NCT03470922). These data suggest the addition of new immunotherapies which target multiple immune checkpoints in cohorts of patients failing PD-1 inhibitor therapy improves outcomes when compared to a switch strategy (Pires Da Silva et al. J Clin Oncol. 38(15_suppl):10005(2020)). However, the risk to patient safety remains a primary concern as Grade 3 or greater AEs occur at levels that exceed that of patients receiving a single immune checkpoint inhibitor alone. For example, in Grade 3 or greater AEs occurred in over 30% of the patients receiving ipilimumab, whether alone or in combination. Patients receiving relatlimab in combination with nivolumab nearly doubled the development of Grade 3 or 4 treatment-related AEs compared with patients receiving nivolumab alone (Tawbi, H. A. et al. Relatlimab and Nivolumab versus Nivolumab in Untreated Advanced Melanoma. N Engl J Med. 386:24-34(2022)).

Taken together, there is significant interest in the development of novel immunotherapeutic agents that can safely increase the anti-tumor response for a substantial proportion of cancer patients that are unable to benefit from current FDA approved therapies.

### SUMMARY OF THE INVENTION

The present invention provides improved methods to treat cancer in a human patient comprising frequent administration to the patient of a high dose of a chimeric poliovirus construct comprising a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame (a “chimeric poliovirus”) intratumorally, optionally in combination with an immune checkpoint inhibitor (ICI) using an induction/maintenance schedule of injections. As provided herein, the induction phase comprises the frequent administration of the chimeric poliovirus at a high dose intratumorally, for example at a total dose of between about 2.0×10⁸ TCID₅₀ and about 5.0×10⁹ TCID₅₀, for example up to about 1.6×10⁹ TCID₅₀, into up to 6-10 tumor lesions once a week for, for example four to ten weeks. In some embodiments, an effective amount of the ICI is administered according to its standard therapeutic protocol, for example once every two weeks, once every three weeks, once every four weeks, or once every 6 weeks, during the induction phase. Following the induction phase, a maintenance phase is initiated wherein the chimeric poliovirus is administered once every two weeks, once every three weeks, once every four weeks, or once every 6 weeks along with an effective amount of an ICI. This dosing protocol shows improved efficacy in the treatment of cancers, including those being TBP as determined, for example, by RECIST 1.1 guidelines. In particular embodiments, the tumor is a melanoma.

In some embodiments, the chimeric poliovirus is administered at a total dose at each administration during the induction and maintenance phases of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the chimeric poliovirus is injected at a total dose of up to 2.0×10¹⁰ TCID₅₀. In some embodiments, the induction phase lasts between 4 weeks and 10 weeks. In some embodiments, the chimeric poliovirus is administered once a week during the induction phase, and the induction phase lasts at least 4 weeks, at least 5 weeks, at least 6 weeks, at least 7 weeks, at least 8 weeks, at least 9 weeks, or up to 10 weeks. In some embodiments, the induction phase lasts 4 weeks. In some embodiments, the induction phase lasts 5 weeks. In some embodiments, the induction phase lasts 6 weeks. In some embodiments, the induction phase lasts 7 weeks. In some embodiments, the induction phase lasts 8 weeks. In some embodiments, the chimeric poliovirus is injected intratumorally into up to 10 tumor lesions per administration. In some embodiments, the chimeric poliovirus is lerapolturev (also known as PVSRIPO). In some embodiments, the tumor is melanoma.

In some embodiments, an effective amount of an ICI can be administered during the induction phase, during the maintenance phase, or during both the induction phase and the maintenance phase. In some embodiments, the ICI is selected from a programmed cell death protein 1 (PD-1) inhibitor, a programmed death-ligand-1 (PD-L1) inhibitor, a cytotoxic T-lymphocyte-associated protein 4 (CTLA-4) inhibitor, a T cell immunoreceptor with Ig and ITIM domains (TIGIT) inhibitor, a T-cell immunoglobulin mucin-3 (TIM-3) inhibitor, or a Lymphocyte-activation gene 3 (LAG-3) inhibitor, a programmed death-ligand 2 (PD-L2) inhibitor, a V-domain Ig suppressor of T-cell activation (VISTA) inhibitor, B7-H3/CD276 inhibitor, an indoleamine 2,3-dioxygenase (IDO) inhibitor, killer immunoglobulin-like receptor (KIR) inhibitor, an carcinoembryonic antigen cell adhesion molecule (CEACAM) inhibitor against molecules such as CEACAM-1, CEACAM-3, and CEACAM-5, a sialic acid-binding immunoglobulin-like lectin 15 (Siglec-15) inhibitor, a CD47 inhibitor, a CD39 inhibitor, or a B and T lymphocyte attenuator (BTLA) protein inhibitor, or a combination thereof.

Importantly, the improved treatment methods described herein increase the efficacy and effectiveness of lerapolturev treatment, including when used in combination with immune checkpoint blockade, including overcoming primary or acquired resistance to previously administered ICIs, and/or reduce or delay the onset of resistance to ICIs, resulting in an extended efficacy of an anti-cancer regimen. The improved treatment methods described herein block tumor cell immune effector signal downregulation to prevent tumor immune escape, and the administration of an effective amount of a chimeric poliovirus construct and an effective amount of an ICI are capable of synergizing to reverse and/or significantly delay the growth of tumors (see, e.g., FIG. 1B-1C, 4A-4E) and/or the development of ICI therapy resistance. The improved treatment methods described herein comprising increased dose frequency and concentration also delay the growth of tumors (FIG. 1D), which is further enhanced when combined with immune checkpoint blockade (see, e.g., FIG. 1F, 1H). Furthermore, the improved treatment methods described herein provide enhanced therapeutic efficacy through the regulation of T cells, including activation of cytotoxic CD8+ T-cell function and maturation into memory CD8+ T-cells. Importantly, the improved treatment methods described herein exhibited substantial viral replication of lerapolturev in macrophages and T cells (see, e.g., FIG. 7A 7E), immune cells that are instrumental in carrying out a targeted anti-tumor immune response.

The improved treatment methods described herein provide significant anti-tumor potency and measurable reductions in tumor progression in humans (see, e.g., FIG. 4A-4B, 5A-5G, 6A-6C). These substantial tumor reductions also coincided with clinically beneficial responses in human patients (see, e.g., Table 4, FIG. 4A-4D, 6A-6B), and in one case, a human patient with a previously unresponsive, recurrent melanoma of 10 years having a complete response with no observable disease remaining (see, e.g., FIG. 5F). The results are seen in patients that include those who previously demonstrated minimal disease burden reduction in lower-dosed and less frequent lerapolturev administration schedules.

Furthermore, the improved treatment methods described herein exhibit an abscopal effect wherein uninjected lesions exhibited clinically meaningful responses as well as those lesions injected, suggestive of a systemic activation of the immune system triggered by the administration of the improved treatment methods as described herein (FIG. 8).

In one aspect, provided herein is a method of treating a human patient having a solid tumor, wherein the treatment comprises:
administering to one or more tumor lesions of the patient an effective amount of a chimeric poliovirus once per week for 7 weeks and once every 3 weeks thereafter; and,
administering to the patient an effective amount of an immune checkpoint inhibitor (ICI) once every 3 weeks. In some embodiments, the method is administered until disease progression or death. In some embodiments, the chimeric poliovirus is lerapolturev. In some embodiments, the ICI is a PD-1 inhibitor. In some embodiments, the PD-1 inhibitor is pembrolizumab. In some embodiments, the chimeric poliovirus is lerapolturev and the ICI is pembrolizumab. In some embodiment, the chimeric poliovirus is administered to at least two, at least three, at least four, at least five, at least six tumor lesions, at least seven tumor lesions, at least eight tumor lesions, at least nine tumor lesions, or up to ten tumor lesions. In some embodiment, the chimeric poliovirus is administered to up to six tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered to up to ten tumor legions. In some embodiments, the chimeric poliovirus is administered at a total dose at each administration of between about 1.6×10⁹ TCID₅₀ to about 2.0×10¹⁰ TCID₅₀, into up to 10 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, a period of at least one week, at least 2 weeks, at least 3 weeks, at least 4 weeks, at least one month, at least 2 months, at least 3 months, at least 4 months, or at least 6 months separate the first administration of the chimeric poliovirus and/or ICI following the end of the 7-weeks. In some embodiments, one or more additional ICIs are administered. In some alternative embodiments, an ICI is not administered. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI following evidence of apparent disease progression. In some embodiments, disease progression is determined by RECIST 1.1 guidelines. In some embodiments, the tumor is a melanoma.

In an alternative aspect, provided herein is a method of treating a human patient having a solid tumor, wherein the treatment comprises:
administering to one or more tumor lesions of the patient an effective amount of a chimeric poliovirus once per week for 7 weeks and once every 3 weeks thereafter; and,
administering to the patient an effective amount of an immune checkpoint inhibitor (ICI) once every 6 weeks. In some embodiments, the method is administered until disease progression or death. In some embodiments, the chimeric poliovirus is lerapolturev. In some embodiments, the ICI is a PD-1 inhibitor. In some embodiments, the PD-1 inhibitor is pembrolizumab. In some embodiments, the chimeric poliovirus is lerapolturev and the ICI is pembrolizumab. In some embodiment, the chimeric poliovirus is administered to at least two, at least three, at least four, at least five, at least six tumor lesions, at least seven tumor lesions, at least eight tumor lesions, at least nine tumor lesions, or up to ten tumor lesions. In some embodiment, the chimeric poliovirus is administered to up to six tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered to up to ten tumor legions. In some embodiments, the chimeric poliovirus is administered at a total dose at each administration of between about 1.6×10⁹ TCID₅₀ to about 2.0×10¹⁰ TCID₅₀, into up to 10 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, a period of at least one week, at least 2 weeks, at least 3 weeks, at least 4 weeks, at least one month, at least 2 months, at least 3 months, at least 4 months, or at least 6 months separate the first administration of the chimeric poliovirus and/or ICI following the end of the 7-weeks. In some embodiments, one or more additional ICIs are administered. In some alternative embodiments, an ICI is not administered. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI following evidence of apparent disease progression. In some embodiments, disease progression is determined by RECIST 1.1 guidelines. In some embodiments, the tumor is a melanoma.

In another alternative aspect, provided herein is a method of treating a human patient having a solid tumor, wherein the treatment comprises:
administering to one or more tumor lesions of the patient an effective amount of a chimeric poliovirus once per week for 7 weeks and once every 4 weeks thereafter, and,
administering to the patient an effective amount of an immune checkpoint inhibitor (ICI) once every 4 weeks. In some embodiments, the method is administered until disease progression or death. In some embodiments, the chimeric poliovirus is lerapolturev. In some embodiments, the ICI is a PD-1 inhibitor. In some embodiments, the PD-1 inhibitor is nivolumab. In some embodiments, the chimeric poliovirus is lerapolturev and the ICI is nivolumab. In some embodiment, the chimeric poliovirus is administered to at least two, at least three, at least four, at least five, at least six tumor lesions, at least seven tumor lesions, at least eight tumor lesions, at least nine tumor lesions, or up to ten tumor lesions. In some embodiment, the chimeric poliovirus is administered to up to six tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered to up to ten tumor legions. In some embodiments, the chimeric poliovirus is administered at a total dose at each administration of between about 1.6×10⁹ TCID₅₀ to about 2.0×10¹⁰ TCID₅₀, into up to 10 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, a period of at least one week, at least 2 weeks, at least 3 weeks, at least 4 weeks, at least one month, at least 2 months, at least 3 months, at least 4 months, or at least 6 months separate the first administration of the chimeric poliovirus and/or ICI following the end of the 7-weeks. In some embodiments, one or more additional ICIs are administered. In some alternative embodiments, an ICI is not administered. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI following evidence of apparent disease progression. In some embodiments, disease progression is determined by RECIST 1.1 guidelines. In some embodiments, the tumor is melanoma.

In another alternative aspect, provided herein is a method of treating a human patient having a solid tumor, wherein the treatment comprises:
administering to one or more tumor lesions of the patient an effective amount of a chimeric poliovirus once per week for 7 weeks and once every 4 weeks thereafter; and,
administering to the patient an effective amount of an immune checkpoint inhibitor (ICI) once every 2 weeks. In some embodiments, the maintenance phase is administered until disease progression or death. In some embodiments, the chimeric poliovirus is lerapolturev. In some embodiments, the ICI is a PD-1 inhibitor. In some embodiments, the PD-1 inhibitor is nivolumab. In some embodiments, the chimeric poliovirus is lerapolturev and the ICI is nivolumab. In some embodiment, the chimeric poliovirus is administered to at least two, at least three, at least four, at least five, at least six tumor lesions, at least seven tumor lesions, at least eight tumor lesions, at least nine tumor lesions, or up to ten tumor lesions. In some embodiment, the chimeric poliovirus is administered to up to six tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered to up to ten tumor legions. In some embodiments, the chimeric poliovirus is administered at a total dose at each administration of between about 1.6×10⁹ TCID₅₀ to about 2.0×10¹⁰ TCID₅₀, into up to 10 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, a period of at least one week, at least 2 weeks, at least 3 weeks, at least 4 weeks, at least one month, at least 2 months, at least 3 months, at least 4 months, or at least 6 months separate the first administration of the chimeric poliovirus and/or ICI following the end of the 7-weeks. In some embodiments, one or more additional ICIs are administered. In some alternative embodiments, an ICI is not administered. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI following evidence of apparent disease progression. In some embodiments, disease progression is determined by RECIST 1.1 guidelines. In some embodiments, the tumor is melanoma.

In one aspect, provided herein is a method of treating a human patient having a solid tumor, wherein the treatment comprises an induction phase and a maintenance phase;
the induction phase comprising:
   administering to one or more tumor lesions of the patient an effective amount of a chimeric poliovirus once per week for up to 7 weeks, and,
   administering an effective amount of an immune checkpoint inhibitor (ICI) once every three weeks during the induction phase;
the maintenance phase comprising:
   administering to one or more tumor lesions of the patient an effective amount of the chimeric poliovirus, wherein the chimeric poliovirus is administered once every three weeks,
   administering an effective amount of the ICI, wherein the ICI is administered every three weeks, and,
wherein the maintenance phase is administered following the cessation of the induction phase. In some embodiments, the maintenance phase is administered until disease progression or death. In some embodiments, the chimeric poliovirus and ICI are administered on the same day during the maintenance phase. In some embodiments, the chimeric poliovirus is lerapolturev. In some embodiments, the ICI is a PD-1 inhibitor. In some embodiments, the PD-1 inhibitor is pembrolizumab. In some embodiments, the chimeric poliovirus is lerapolturev and the ICI is pembrolizumab. In some embodiments, the chimeric poliovirus is administered to at least two, at least three, at least four, at least five, at least six, at least seven, at least eight, at least nine, or up to ten tumor lesions. In some embodiments, the chimeric poliovirus is administered to up to six tumor lesions. In some embodiments, the chimeric poliovirus is administered to up to ten tumor lesions. In some embodiments, the chimeric poliovirus is administered at a total dose at each administration of between about 2.0×10⁸ TCID₅₀ and about 2.0×10¹⁰ TCID₅₀, into up to 10 tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and about 6 weeks prior to the first administration of the chimeric poliovirus. In some embodiments, one or more additional ICIs are administered during the induction phase and/or maintenance phase. In some alternative embodiments, an ICI is not administered during either the induction phase or the maintenance phase. In some alternative embodiments, an ICI is not administered during the induction phase. In some alternative embodiments, an ICI is not administered during the maintenance phase. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI during the induction phase and/or maintenance phase following evidence of apparent disease progression. In some embodiments, disease progression is determined by RECIST 1.1 guidelines. In some embodiments, the tumor is melanoma.

In one alternative aspect, provided herein is a method of treating a human patient having a solid tumor, wherein the treatment comprises an induction phase and a maintenance phase;
the induction phase comprising:
   administering to one or more tumor lesions of the patient an effective amount of a chimeric poliovirus once per week for up to 7 weeks, and,
   administering an effective amount of an immune checkpoint inhibitor (ICI) once every four weeks during the induction phase;
the maintenance phase comprising:
   administering to one or more tumor lesions of the patient an effective amount of the chimeric poliovirus, wherein the chimeric poliovirus is administered once every four weeks, and,
   administering an effective amount of the ICI, wherein the ICI is administered every four weeks, and,
wherein the maintenance phase is administered following the cessation of the induction phase. In some embodiments, the maintenance phase is administered until disease progression or death. In some embodiments, the chimeric poliovirus and ICI are administered on the same day during the maintenance phase. In some embodiments, the chimeric poliovirus is lerapolturev. In some embodiments, the ICI is a PD-1 inhibitor. In some embodiments, the PD-1 inhibitor is nivolumab. In some embodiments, the chimeric poliovirus is lerapolturev and the ICI is nivolumab. In some embodiment, the chimeric poliovirus is administered to at least two, at least three, at least four, at least five, at least six tumor lesions, at least seven tumor lesions, at least eight tumor lesions, at least nine tumor lesions, or up to ten tumor lesions. In some embodiment, the chimeric poliovirus is administered to up to six tumor lesions. In some embodiments, the chimeric poliovirus is administered at a total dose at each administration of between about 2.0×10⁸ TCID₅₀ and about 2.0×10¹⁰ TCID₅₀, into up to 10 tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, one or more additional ICIs are administered during the induction phase and/or maintenance phase. In some alternative embodiments, an ICI is not administered during either the induction phase or the maintenance phase. In some alternative embodiments, an ICI is not administered during the induction phase. In some alternative embodiments, an ICI is not administered during the maintenance phase. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI during the induction phase and/or maintenance phase following evidence of apparent disease progression. In some embodiments, disease progression is determined by RECIST 1.1 guidelines. In some embodiments, the tumor is melanoma.

In one alternative aspect, provided herein is a method of treating a human patient having a solid tumor, wherein the treatment comprises an induction phase and a maintenance phase;
the induction phase comprising:
   administering to one or more tumor lesions of the patient an effective amount of a chimeric poliovirus once per week for up to 7 weeks, and,
   administering an effective amount of an immune checkpoint inhibitor (ICI) once every two weeks during the induction phase;
the maintenance phase comprising:
   administering to one or more tumor lesions of the patient an effective amount of the chimeric poliovirus, wherein the chimeric poliovirus is administered once every four weeks, and,
   administering an effective amount of the ICI, wherein the ICI is administered every two weeks, and,
wherein the maintenance phase is administered following the cessation of the induction phase. In some embodiments, the maintenance phase is administered until disease progression or death. In some embodiments, the chimeric poliovirus is lerapolturev. In some embodiments, the ICI is a PD-1 inhibitor. In some embodiments, the PD-1 inhibitor is nivolumab. In some embodiments, the chimeric poliovirus is lerapolturev and the ICI is nivolumab. In some embodiment, the chimeric poliovirus is administered to at least two, at least three, at least four, at least five, at least six tumor lesions, at least seven tumor lesions, at least eight tumor lesions, at least nine tumor lesions, or up to 10 tumor lesions. In some embodiment, the chimeric poliovirus is administered to up to six tumor lesions. In some embodiments, the chimeric poliovirus is administered at a total dose at each administration of between about 2.0×10⁸ TCID₅₀ and about 2.0×10¹⁰ TCID₅₀, into up to 10 tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, one or more additional ICIs are administered during the induction phase and/or maintenance phase. In some alternative embodiments, an ICI is not administered during either the induction phase or the maintenance phase. In some alternative embodiments, an ICI is not administered during the induction phase. In some alternative embodiments, an ICI is not administered during the maintenance phase. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI during the induction phase and/or maintenance phase following evidence of apparent disease progression. In some embodiments, disease progression is determined by RECIST 1.1 guidelines. In some embodiments, the tumor is melanoma.

In one alternative aspect, provided herein is a method of treating a human patient having a solid tumor, wherein the treatment comprises an induction phase and a maintenance phase;
the induction phase comprising:
   administering to one or more tumor lesions of the patient an effective amount of a chimeric poliovirus once per week for 7 weeks, and,
   administering an effective amount of an immune checkpoint inhibitor (ICI) once every 6 weeks during the induction phase;
the maintenance phase comprising:
   administering to one or more tumor lesions of the patient an effective amount of the chimeric poliovirus, wherein the chimeric poliovirus is administered once every three weeks, and,
   administering an effective amount of the ICI, wherein the ICI is administered every 6 weeks, and,
wherein the maintenance phase is administered following the cessation of the induction phase. In some embodiments, the maintenance phase is administered until disease progression or death. In some embodiments, the chimeric poliovirus is lerapolturev. In some embodiments, the ICI is a PD-1 inhibitor. In some embodiments, the PD-1 inhibitor is nivolumab. In some embodiments, the chimeric poliovirus is lerapolturev and the ICI is nivolumab. In some embodiment, the chimeric poliovirus is administered to at least two, at least three, at least four, at least five, at least six tumor lesions, at least seven tumor lesions, at least eight tumor lesions, at least nine tumor lesions, or up to ten tumor lesions. In some embodiment, the chimeric poliovirus is administered to up to six tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered to up to ten tumor legions. In some embodiments, the chimeric poliovirus is administered at a total dose at each administration of between about 1.6×10⁹ TCID₅₀ to about 2.0×10¹⁰ TCID₅₀, into up to 10 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, one or more additional ICIs are administered during the induction phase and/or maintenance phase. In some alternative embodiments, an ICI is not administered during either the induction phase or the maintenance phase. In some alternative embodiments, an ICI is not administered during the induction phase. In some alternative embodiments, an ICI is not administered during the maintenance phase. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI during the induction phase and/or maintenance phase following evidence of apparent disease progression. In some embodiments, disease progression is determined by RECIST 1.1 guidelines. In some embodiments, the tumor is melanoma.

In another alternative aspect, provided herein is a method of treating a human patient having a solid tumor, wherein the treatment comprises an induction phase and a maintenance phase;
the induction phase comprising two 21-day cycles, each 21-day cycle comprising:
   administering to one or more tumor lesions of the patient an effective amount of a chimeric poliovirus on days 1, 8, and 15 of each 21-day induction cycle, and,
   administering an effective amount of an ICI on day 1 of each 21-day induction cycle;
the maintenance phase comprising one or more 21-day cycles, each 21-day cycle comprising:
   administering to one or more tumor lesions of the patient an effective amount of the chimeric poliovirus on day 1 of each 21-day maintenance cycle, and,
   administering an effective amount of an ICI on day 1 of each 21-day maintenance cycle.
wherein the maintenance phase is administered following the cessation of the induction phase. In some embodiments, the chimeric poliovirus is lerapolturev. In some embodiments, the ICI is a PD-1 inhibitor. In some embodiments, the PD-1 inhibitor is pembrolizumab. In some embodiments, the chimeric poliovirus is lerapolturev and the ICI is pembrolizumab. In some embodiment, the chimeric poliovirus is administered to at least two, at least three, at least four, at least five, at least six tumor lesions, at least seven tumor lesions, at least eight tumor lesions, at least nine tumor lesions, or up to ten tumor lesions. In some embodiments, the chimeric poliovirus is administered to up to six tumor lesions. In some embodiments, the chimeric poliovirus is administered to up to ten tumor lesions. In some embodiments, the chimeric poliovirus is administered at a total dose at each administration of between about 2.0×10⁸ TCID₅₀ and about 2.0×10¹⁰ TCID₅₀, into up to 10 tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, one or more additional ICIs are administered during the induction phase and/or maintenance phase. In some alternative embodiments, an ICI is not administered during either the induction phase or the maintenance phase. In some alternative embodiments, an ICI is not administered during the induction phase. In some alternative embodiments, an ICI is not administered during the maintenance phase. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI during the induction phase and/or maintenance phase following evidence of apparent disease progression. In some embodiments, disease progression is determined by RECIST 1.1 guidelines. In some embodiments, the tumor is melanoma.

In another alternative aspect, provided herein is a method of treating a human patient having a solid tumor, wherein the treatment comprises an induction phase and a maintenance phase;
the induction phase comprising a first 28-day cycle and a second 28-day cycle,
the first 28-day cycle comprising:
   administering to one or more tumor lesions of the patient an effective amount of a chimeric poliovirus on days 1, 8, 15, and 22 of the first 28-day induction cycle,
   administering an effective amount of an ICI on day 1 of the first 28-day induction cycle;
the second 28-day cycle comprising:
   administering to one or more tumor lesions of the patient an effective amount of a chimeric poliovirus on days 1, 8, and 15 of the second 28-day induction cycle,
   administering an effective amount of an ICI on day 1 of the second 28-day induction cycle;
the maintenance phase comprising one or more 28-day cycles, each 28-day cycle comprising:
   administering to one or more tumor lesions of the patient an effective amount of the chimeric poliovirus on day 1 of each 28-day maintenance cycle,
   administering an effective amount of an immune checkpoint inhibitor (ICI) on day 1 of each 28-day maintenance cycle, and,
wherein the maintenance phase is administered following the cessation of the induction phase. In some embodiments, the chimeric poliovirus is lerapolturev. In some embodiments, the ICI is a PD-1 inhibitor. In some embodiments, the PD-1 inhibitor is nivolumab. In some embodiments, the chimeric poliovirus is lerapolturev and the ICI is nivolumab. In some embodiment, the chimeric poliovirus is administered to at least two, at least three, at least four, at least five, at least six, at least seven, at least eight, at least nine, or up to ten tumor lesions. In some embodiments, the chimeric poliovirus is administered to up to six tumor lesions. In some embodiments, the chimeric poliovirus is administered to up to ten tumor lesions. In some embodiments, the chimeric poliovirus is administered at a total dose at each administration of between about 2.0×10⁸ TCID₅₀ and about 2.0×10¹⁰ TCID₅₀, into up to 10 tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, one or more additional ICIs are administered during the induction phase and/or maintenance phase. In some alternative embodiments, an ICI is not administered during either the induction phase or the maintenance phase. In some alternative embodiments, an ICI is not administered during the induction phase. In some alternative embodiments, an ICI is not administered during the maintenance phase. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI during the induction phase and/or maintenance phase following evidence of apparent disease progression. In some embodiments, disease progression is determined by RECIST 1.1 guidelines. In some embodiments, the tumor is melanoma.

In another alternative aspect, provided herein is a method of treating a human patient having a solid tumor, wherein the treatment comprises an induction phase and a maintenance phase;
the induction phase comprising two 21-day cycles,
   the first 21-day cycle comprising:
   administering to one or more tumor lesions of the patient an effective amount of a chimeric poliovirus on days 1, 8, and 15 of each 21-day induction cycle, and,
   administering an effective amount of an immune checkpoint inhibitor (ICI) on day 1 of each 21-day induction cycle, and,
   the second 21-day cycle comprising:
   administering to one or more tumor lesions of the patient an effective amount of a chimeric poliovirus on days 1, 8, and 15 of each 21-day induction cycle;
the maintenance phase comprising one or more 21-day maintenance cycles, comprising:
   administering to one or more tumor lesions of the patient an effective amount of the chimeric poliovirus on day 1 of each 21-day maintenance cycle,
   administering an effective amount of an immune checkpoint inhibitor (ICI) on day 1 of every other 21-day maintenance cycle and,
wherein, the maintenance phase is administered following the cessation of the induction phase. In some embodiments, the chimeric poliovirus is lerapolturev. In some embodiments, the ICI is a PD-1 inhibitor. In some embodiments, the PD-1 inhibitor is pembrolizumab. In some embodiments, the chimeric poliovirus is lerapolturev and the ICI is pembrolizumab. In some embodiment, the chimeric poliovirus is administered to at least two, at least three, at least four, at least five, at least six tumor lesions, at least seven tumor lesions, at least eight tumor lesions, at least nine tumor lesions, or up to ten tumor lesions. In some embodiments, the chimeric poliovirus is administered to up to six tumor lesions. In some embodiments, the chimeric poliovirus is administered to up to ten tumor lesions. In some embodiments, the chimeric poliovirus is administered at a total dose at each administration of between about 2.0×10⁸ TCID₅₀ and about 2.0×10¹⁰ TCID₅₀, into up to 10 tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, one or more additional ICIs are administered during the induction phase and/or maintenance phase. In some alternative embodiments, an ICI is not administered during either the induction phase or the maintenance phase. In some alternative embodiments, an ICI is not administered during the induction phase. In some alternative embodiments, an ICI is not administered during the maintenance phase. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI during the induction phase and/or maintenance phase following evidence of apparent disease progression. In some embodiments, disease progression is determined by RECIST 1.1 guidelines. In some embodiments, the tumor is melanoma.

In another aspect, provided herein is a method of treating a human patient having a solid tumor, wherein the treatment comprises an induction phase and a maintenance phase;
the induction phase comprising a first 28-day cycle and a second 28-day cycle,
the first 28-day cycle comprising:
   administering to one or more tumor lesions of the patient an effective amount of a chimeric poliovirus on days 1, 8, 15, and 22 of the first 28-day induction cycle,
   administering an effective amount of an immune checkpoint inhibitor (ICI) on day 1 and day 15 of the first 28-day induction cycle;
the second 28-day cycle comprising:
   administering to one or more tumor lesions of the patient an effective amount of a chimeric poliovirus on days 1, 8, and 15 of the second 28-day induction cycle,
   administering an effective amount of an immune checkpoint inhibitor (ICI) on day 1 and day 15 of the second 28-day induction cycle;
the maintenance phase comprising one or more 28-day cycles, each 28-day cycle comprising:
   administering to one or more tumor lesions of the patient an effective amount of the chimeric poliovirus on day 1 of each 28-day maintenance cycle,
   administering an effective amount of an immune checkpoint inhibitor (ICI) on day 1 and day 15 of each 28-day maintenance cycle, and,
wherein the maintenance phase is administered following the cessation of the induction phase. In some embodiments, the chimeric poliovirus is lerapolturev. In some embodiments, the ICI is a PD-1 inhibitor. In some embodiments, the PD-1 inhibitor is nivolumab. In some embodiments, the chimeric poliovirus is lerapolturev and the ICI is nivolumab. In some embodiment, the chimeric poliovirus is administered to at least two, at least three, at least four, at least five, at least six tumor lesions, at least seven tumor lesions, at least eight tumor lesions, at least nine tumor lesions, or up to ten tumor lesions. In some embodiment, the chimeric poliovirus is administered to up to six tumor lesions. In some embodiments, the chimeric poliovirus is administered to up to ten tumor lesions. In some embodiments, the chimeric poliovirus is administered at a total dose at each administration of between about 2.0×10⁸ TCID₅₀ and about 2.0×10¹⁰ TCID₅₀, into up to 10 tumor lesions. In some embodiments, the chimeric poliovirus is administered at a total dose at each administration of between about 2.0×10⁸ TCID₅₀ and about 5.0×10⁹ TCID₅₀, for example between about 2.67×10⁸ TCID₅₀ to about 1.6×10⁹ TCID₅₀, into up to 6 tumor lesions. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. In some embodiments, the patient is administered a poliovirus vaccine between about 1 week and 6 weeks prior to the first administration of the chimeric poliovirus. In some embodiments, one or more additional ICIs are administered during the induction phase and/or maintenance phase. In some alternative embodiments, an ICI is not administered during either the induction phase or the maintenance phase. In some alternative embodiments, an ICI is not administered during the induction phase. In some alternative embodiments, an ICI is not administered during the maintenance phase. In some embodiments, the patient's solid tumor, prior to the initiation of the induction phase, has progressed following receiving an ICI. In some embodiments, the patient's solid tumor, prior to the initiation of the induction phase, has progressed following receiving a MEK inhibitor. In some embodiments, the patient's solid tumor, prior to the initiation of the induction phase, has progressed following receiving a BRAK inhibitor. In some embodiments, the patient is administered one or more doses of lerapolturev and, optionally, an ICI during the induction phase and/or maintenance phase following evidence of apparent disease progression. In some embodiments, disease progression is determined by RECIST 1.1 guidelines. In some embodiments, the tumor is melanoma.

In some embodiments, the chimeric poliovirus administered in the methods described herein is lerapolturev, also known as PVSRIPO, a chimeric poliovirus construct comprising a Sabin type I strain of poliovirus with a human rhinovirus 2 (HRV2) internal ribosome entry site (IRES) in said poliovirus' 5′ untranslated region between said poliovirus' cloverleaf and said poliovirus' open reading frame, thus eliminating its neurovirulence (Gromeier et al. Proc Natl Acad Sci. 93(6):2370-5(1996); Gromeier et al. Virology. 273(2):248-57(2000)). The nucleic acid sequence of lerapolturev is provided in SEQ ID NO:1. In some embodiments, the chimeric poliovirus administered according to the methods provided herein has a nucleic acid sequence of SEQ ID NO:1, or a nucleic acid sequence at least 80%, at least 85%, at least 90%, at least 95%, at least 96%, at least 97%, at least 98%, or at least 99% identical thereto.

In some embodiments, the chimeric poliovirus is lerapolturev, and is administered at a fixed concentration of between about 4.0×10⁸ TCID₅₀/ml and about 6.0×10⁸ TCID₅₀/ml. In some embodiments, lerapolturev is administered at about 5.33×10⁸ TCID₅₀/ml. In some embodiments, the maximum volume injected at an individual treatment visit will be between about 2.5 ml and about 3.5 ml. In some embodiments, the maximum volume injected at an individual treatment visit is about 3 ml. In some embodiments, the maximum lerapolturev dose administered is about 1.6×10⁹ TCID₅₀. In some embodiments, the maximum lerapolturev dose administered is about 1.0×10¹⁰ TCID₅₀. In some embodiments, the minimum injection volume is 0.5 ml. In some embodiments, the minimum lerapolturev dose administered at an individual treatment visit is about 2.67×10⁸ TCID₅₀. As provided herein, up to 6 lesions may be injected at an individual treatment visit. As provided herein, up to 10 lesions may be injected at an individual treatment visit. In some embodiments, the chimeric poliovirus is administered at a total dose at each administration of between about 1.0×10⁹ TCID₅₀ and about 2.0×10⁹ TCID₅₀. In some embodiments, the chimeric poliovirus is administered at a total dose at each administration of about 1.6×10⁹ TCID₅₀. It is possible that more than 1 syringe may be required to treat a single lesion, due to large lesion size, for example as provided in Table 2 below.

Because of the near-universal vaccination against the poliovirus, the use of lerapolturev is capable of recalling a host-derived immune vaccine generated response. Furthermore, lerapolturev enters cells via the poliovirus receptor, CD155, which is a cell adhesion molecule of the Ig-like superfamily expressed during embryonic development (Gromeier et al. Virology. 273(2):248-57(2000); Brown et al. Sci Transl Med. 9(408)(2017); Holl et al. Oncotarget. 7(48):79828-41(2016)), and plays an important role in cell migration, invasion, and metastasis. As such, CD155 is broadly expressed in a variety of solid tumors, including melanoma and GBM (Chandramohan et al. Arch Pathol Lab Med. 141(12):1697-1704(2017)). Upon binding CD155, lerapolturev viral

RNA enters and replicates within the cytoplasm to initiate direct viral cytotoxicity in tumor cells through engagement of anti-viral interferon response (Brown et al. Sci Transl Med. 9(408)(2017)). Lerapolturev is directly cytotoxic to neoplastic cells, which offers ideal conditions for viral IRES-mediated ribosome recruitment due to the unrestrained protein synthesis required in cancer cells (Brown et al. Cancer. 120(21):3277-86(2014)). Lerapolturev also induces non-lethal infection of antigen presenting dendritic cells (DC) to increase immune effector responses directed against tumor neoantigens (Brown et al. Sci Transl Med. 9(408)(2017)), thus driving a secondary immune response. The principal elements determining lerapolturev tumor tropism, tumor-specific cell killing, neuronal incompetence/safety, and immunogenicity are well established empirically (Brown et al. Sci Transl Med. 9(408)(2017); Brown et al. J Virol. 88(22):13149-60(2014)). Notably, lerapolturev infection in dendritic cells significantly increases PD-L1 expression (Brown et al. Nature Commun, 12(1858):1-16(2021)), providing, in combination with ICI therapy, the ability of the immune system to specifically recognize and destroy a tumor.

Advantageously, the chimeric poliovirus/ICI therapeutic protocol provided herein can be repeatedly administered, as needed. For example, the chimeric poliovirus (e.g., lerapolturev) can be repeatedly administered to, for example, melanoma metastatic lesions intratumorally or other suitable delivery areas, in combination with PD-1 inhibitors or other ICIs (see, e.g., FIG. 1A-B, FIG. 4A-D, FIG. 5A-G, FIG. 6A-C). In some embodiments, the chimeric poliovirus/ICI therapeutic protocol provided herein can provide anti-tumor efficacy in both injected and non-injected lesions (see, e.g., FIG. 4D, FIG. 6B-C), referred to as an abscopal response. In some embodiments, the chimeric poliovirus/ICI therapeutic protocol provided herein can achieve partial and/or complete responses in lesions of patients naïve to previous cancer treatment and those patients refractory to previous therapies (see, e.g., FIG. 4A-D, FIG. 5A-G, FIG. 6A-C). In some embodiments, the methods described herein can be used to treat a solid tumor comprising administering the chimeric poliovirus to between 1 and 6 lesions. In some embodiments, the methods described herein can be used to treat a solid tumor comprising administering the chimeric poliovirus to between 6 and 10 lesions. In some embodiments, the chimeric poliovirus is administered to at least 1 lesion, at least 2 lesions, at least 3 lesions, at least 4 lesions, at least 5 lesions, at least 6 lesions, at least 7 lesions, at least 8 lesions, at least 9 lesions, or 10 or more lesions per administration.

The ability to frequently administer the chimeric poliovirus (e.g., lerapolturev) at high doses in combination with cycling ICI therapy represents a significant advance in cancer treatment regimens, as a major concern with the therapeutic use of ICIs is the significant population of patients with primary or acquired resistance to ICI therapy. As provided herein, the induction phase is administered so that the chimeric poliovirus is frequently administered, for example once a week during the induction, which is followed by additional administrations of the chimeric poliovirus during the maintenance phase. In some embodiments, the maintenance phase comprises between 2-10 cycles. In some embodiments, the maintenance phase comprises up to 10 cycles. In some embodiments, the maintenance phase comprises more than 10 cycles. Unexpectedly, data from non-clinical animal studies suggest that treatment regimens with more frequent lerapolturev injections at higher doses may provide even higher anti-tumor potency when combined with ICI therapy than previous therapy regimes, without an increase in significant adverse events.

The methods described herein can additionally include the repeat administration of a chimeric poliovirus construct as a monotherapy. In one aspect, provided herein is a method of treating a human patient having a solid tumor, wherein the treatment comprises an induction phase and a maintenance phase;

the induction phase comprising:
administering to one or more tumor lesions of the patient an effective amount of a chimeric poliovirus once per a 7-day induction cycle, wherein the 7-day induction cycle is administered one or more times;
the maintenance phase comprising:
administering to one or more tumor lesions of the patient an effective amount of the chimeric poliovirus, wherein the chimeric poliovirus is administered once per a maintenance cycle selected from a 7-day cycle, a 14-day cycle, a 21-day cycle, a 28-day cycle, a 35-day cycle, or a 42-day cycle.
   In some embodiments, the maintenance phase is administered following the cessation of the induction phase. In some embodiments, the chimeric poliovirus construct is lerapolturev. In some embodiments, the administration can be scheduled as an induction/maintenance dosage regime. As provided herein, the induction phase is administered so that the chimeric poliovirus is frequently administered, for example once a week during the induction. In some embodiments, the induction cycle is repeated 2-10 times, or more than 10 times. In some embodiments, the induction phase is followed by additional administrations of the chimeric poliovirus during a maintenance phase. In some embodiments, the maintenance phase comprises maintenance cycles repeated 2-10 times, or more than 10 times. In some embodiments, the methods described herein can be used to treat a solid tumor comprising administering a chimeric poliovirus construct at a dose of between about 2.67.0×10⁸ to about 1.0×10¹⁰ TCID₅₀. In some embodiments, the methods described herein can be used to treat a solid tumor comprising administering a chimeric poliovirus construct at a dose of between about 8.0×10⁸ to about 1.0×10⁹, about 1.0×10⁹ to about 3.0×10⁹, about 3.0×10⁹ to about 5.0×10⁹ TCID₅₀, about 5.0×10⁹ to about 7.0×10⁹ TCID₅₀, or about 7.0×10⁹ to about 2.0×10¹⁰ TCID₅₀. In some embodiments, the methods described herein can be used to treat a solid tumor comprising administering a chimeric poliovirus construct at a dose of about 1.6×10⁹ TCID₅₀ per administration. In some embodiments, the chimeric poliovirus is administered at a total dose at each administration of between about 1.0×10⁹ TCID₅₀ and about 2.0×10⁹ TCID₅₀. In some embodiments, the methods described herein can be used to treat a solid tumor comprising administering the chimeric poliovirus to between 1 and 10 lesions. In some embodiments, the chimeric poliovirus is administered to at least 1 lesion, at least 2 lesions, at least 3 lesions, at least 4 lesions, at least 5 lesions, at least 6 lesions, at least 7 lesions, at least 8 lesions, at least 9 lesions, or up to 10 lesions per administration. In some embodiments, the tumor is a melanoma.

The methods described herein can be used to treat a human patient having a solid tumor, including, but not limited to glioblastoma multiforme (GBM), astrocytoma, oligodendroglioma, astro-oligodendroglioma, renal cell carcinoma, prostate cancer, bladder cancer, esophageal cancer, stomach cancer, pancreas cancer, colorectal cancer, liver cancer, gall bladder cancer, breast cancer, medulloblastoma, lung cancer, head and neck squamous cell carcinoma (HNSCC), melanoma, ovarian cancer, or sarcoma. In some embodiments, the patient's tumor is PD-1/PD-L1 inhibitor resistant. In some embodiments, the solid tumor is a locally advanced, metastatic, or recurrent tumor. In some embodiments, the patient is administered one or more doses of lerapolturev during the induction phase and/or maintenance phase following evidence of apparent disease progression. In some embodiments, disease progression is determined by RECIST 1.1 guidelines.

In some embodiments, the cancer to be treated according to the methods described herein are PD-1 and/or PD-L1 inhibitor refractory or resistant. For example, in some embodiments, the patient has confirmed progression of disease (PD) while receiving at least 6 weeks (>1 dose) of an FDA-approved PD-1/PD-L1 inhibitor therapy (as monotherapy or in combination) for the treatment of the cancer.

The methods described herein can include the administration of one or more ICIs. Suitable ICIs for use in the methods described herein include, but are not limited to, a programmed cell death-1 (PD-1) inhibitor, a programmed cell death-ligan 1 (PD-L1) inhibitor, a cytotoxic T-lymphocyte-associated protein 4 (CTLA-4) inhibitor, a lymphocyte-activation gene 3 (LAG-3) inhibitor, a T-cell immunoglobulin mucin-3 (TIM-3) inhibitor, a T cell immunoreceptor with Ig and ITIM domains (TIGIT) a programmed death-ligand 2 (PD-L2), a V-domain Ig suppressor of T-cell activation (VISTA) inhibitor, a B7-H3/CD276 inhibitor, an indoleamine 2,3-dioxygenase (IDO) inhibitor, a killer immunoglobulin-like receptor (KIR) inhibitor, a carcinoembryonic antigen cell adhesion molecule (CEACAM) inhibitor against molecules such as CEACAM-1, CEACAM-3, and CEACAM-5, a sialic acid-binding immunoglobulin-like lectin 15 (Siglec-15) inhibitor, a CD47 inhibitor, a CD39 inhibitor, or a B and T lymphocyte attenuator (BTLA) protein inhibitor, or a combination thereof. In some embodiments, the ICI administered is a PD-1 inhibitor. In some embodiments, the PD-1 inhibitor is selected from nivolumab, pembrolizumab, pidilizumab, AMP-224, sasanlimab, spartalizumab, cemiplimab, retifanlimab, tislelizumab, camrelizumab, CS1003, or dostarlimab. In some embodiments, the PD-1 inhibitor is pembrolizumab. In some embodiments, the pembrolizumab is administered in a dose of about 200 mg. In some embodiments, the pembrolizumab is administered in a dose of about 400 mg. In some embodiments, the PD-1 inhibitor is nivolumab. In some embodiments, the nivolumab is administered in a dose of about 480 mg. In some embodiments, the nivolumab is administered in a dose of about 360 mg. In some embodiments, nivolumab is administered in a dose of about 240 mg. In some embodiments, the ICI administered is a PD-L1 inhibitor. In some embodiments, the PD-L1 inhibitor is selected from atezolizumab, durvalumab, avelumab, envafolimab, BMS-936559, lodapolimab, cosibelimab, sugemalimab, adebrelimab, CBT-502, or BGB-A333. In some embodiments, the ICI administered is a CTLA-4 inhibitor. In some embodiments, the CTLA-4 inhibitor is selected from ipilimumab or tremelimumab. In some embodiments, the ICI administered is a LAG-3 inhibitor. In some embodiments, the LAG-3 inhibitor is selected from relatlimab, GSK2831781, eftilagimod alpha, leramilimab, MK-4280, REGN3767, TSR-033, BI754111, Sym022, tebotelimab, FS118, LAG-526, favezelimab, CB213, SNA-03, INCAGN02385, RO7247669, IBI323, EMB-02, or AVA-0017. In some embodiments, the TIM-3 inhibitor is selected from TSR-022, MBG453, Sym023, INCAGN2390, LY3321367, BMS-986258, SHR-1702, RO7121661, sabatolimab, cobolimab, RG7769, MAS-825, BGBA425, AZD7789, TQB2618, or NB002. In some embodiments, the ICI administered is a TIGIT inhibitor. In some embodiments, the TIGIT inhibitor is selected from MK-7684, etigilimab/OMP-313 M32, tiragolumab/MTIG7192A/RG-6058, BMS-986207, AB-154, ASP-8374, Vibostolimab, AZD2936, ASP8374, Domvanalimab, IBI939, Ociperlimab, EOS884448, SEA-TGT, COM902, MPH-313, M6223, HLX53, JS006, mAb-7, SHR-1708, BAT6005, GS02, RXI-804, NB6253, ENUM009, CASC-674, AJUD008, or AGEN1777. In some embodiments, the ICI is opdualag. In some embodiments, the ICI is administered no more than 24 hours before or after the administration of the chimeric poliovirus construct. In some embodiments, the ICI is administered no more than 48 hours before or after the administration of the chimeric poliovirus construct. In some embodiments, the ICI is administered no more than 6 hours before or after the administration of the chimeric poliovirus construct. In some embodiments, the ICI is administered no more than 1 hour before or after the administration of the chimeric poliovirus construct. In some embodiments, the ICI is administered no more than 30 minutes before or after the administration of the chimeric poliovirus construct. In some embodiments, the ICI is administered simultaneously with the administration of the chimeric poliovirus construct. In some embodiments, the method further comprises administering to the patient an effective amount of a chemotherapeutic agent. In some embodiments, the method further comprises administering to the patient one or more different ICIs.

In some embodiments, prior to administration of the first dose of the chimeric poliovirus, a patient is first administered a boost immunization of a poliovirus vaccine, for example, at least 1 week, but less than 6 weeks, prior to day 1 of the first induction phase cycle. Suitable poliovirus vaccines for administration prior to the initiation of the induction phase include trivalent IPOL® (Sanofi-Pasteur SA).

In some embodiments, the methods provided herein further comprise administering an anti-cancer therapy. In some embodiments, the anti-cancer therapy is selected from chemotherapy, immunotherapy, viral therapy, or radiation therapy. In some embodiments, the immunotherapy comprises an interleukin 2 (IL-2) drug or prodrug. In some embodiments, the IL-2 drug or prodrug is bempegaldesleukin (NKTR-214).

The administration of a treatment protocol described herein provides enhanced anti-tumor efficacy in patients. In some embodiments, the administration of a treatment protocol described herein provides improved progression free survival (PFS) and/or overall survival (OS) compared to a patient receiving an ICI alone. In some embodiments, an improvement in OS is observed. In some embodiments, an improvement in PFS is observed.

### BRIEF DESCRIPTION OF THE DRAWINGS

FIGS. 1A-1I show combination therapy comprising multiple administrations of PVSRIPO injections with anti-PD-1 antibody reduces tumor size in both injected and non-injected lesions.

FIG. 1A is an experiment schematic. B16-OVA melanoma model mice (n=80) were randomized into different treatment groups (n=10, each) comprising intraperitoneal injections of either anti-PD-1 murine antibody or IgG antibody control, and intratumoral injections of either one or two doses of PVSRIPO or mock control. Tumor volume was measured in both intratumoral injected lesions and non-injected lesions.

FIG. 1B is a box plot showing changes in size of injected tumors. The y-axis is the injected tumor size at day 9 relative to baseline injected tumor size and the x-axis is the different treatment groups.

FIG. 1C is a box plot showing changes in size of non-injected tumors. The y-axis is the non-injected tumor size at day 9 relative to baseline non-injected tumor size and the x-axis is the different treatment groups.

FIG. 1D is a line plot showing changes in injected tumor size progression over the length of the experiment. The y-axis is the percent of baseline injected tumor size and the x-axis is the days post-first dose of PVSRIPO. The arrows represent the timing of PVSRIPO doses (1 or 2).

FIG. 1E is a line plot showing changes in non-injected tumor size progression over the length of the experiment. The y-axis is the percent of baseline non-injected tumor size and the x-axis is the days post-first dose of PVSRIPO. The arrows represent the timing of PVSRIPO doses (1 or 2).

FIG. 1F is a line plot showing changes in injected tumor size progression over the length of the experiment when either anti-PD1 antibody or IgG treatment is combined with PVSRIPO. The y-axis is the percent of baseline injected tumor size and the x-axis is the days post-first dose of PVSRIPO. The arrows represent the timing of PVSRIPO doses (1 or 2).

FIG. 1G is a line plot showing changes in injected tumor size progression over the length of the experiment when either anti-PD1 antibody or IgG treatment is combined with PVSRIPO. The y-axis is the percent of baseline non-injected tumor size and the x-axis is the days post-first dose of PVSRIPO. The arrows represent the timing of PVSRIPO doses (1 or 2).

FIG. 1H is a line plot showing changes in injected tumor size progression over the length of the experiment when anti-PD1 antibody treatment is combined with PVSRIPO or MOCK treatments. The y-axis is the percent of baseline injected tumor size and the x-axis is the days post-first dose of PVSRIPO. The arrows represent the timing of PVSRIPO doses (1 or 2).

FIG. 1I is a line plot showing changes in non-injected tumor size progression over the length of the experiment when either anti-PD1 antibody or IgG treatment is combined with PVSRIPO. The y-axis is the percent of baseline injected tumor size and the x-axis is the days post-first dose of PVSRIPO. The arrows represent the timing of PVSRIPO doses (1 or 2).

FIGS. 2A-2D is a collection of tables illustrating the dose regimen schedules for lerapolturev+pembrolizumab and lerapolturev+nivolumab or other cycling immune checkpoint inhibitors (ICIs). The schedules are conducted in two separate phases: 1) an Induction phase wherein lerapolturev is administered weekly concomitant with ICI therapy cycling at different frequencies; and 2) a Maintenance phase wherein lerapolturev is administered according to the cycling of the ICI therapy. Lerapolturev is administered weekly for 7 weeks to start the dose regimen. Following the 7^{th} weekly lerapolturev injection, lerapolturev is administered Q3W when in combination with a Q3W or Q6W ICI (e.g., pembrolizumab) or administered Q4W when in combination with a Q2W or Q4W ICI (e.g., nivolumab).

FIG. 2A is a table illustrating different dose regimen schedules combining lerapolturev (L) and either Pembrolizumab (P) cycling at either every 3 weeks (3 WK) or 6 weeks (6 WK) or Nivolumab (N) cycling at either 4 weeks (4 WK) or 2 weeks (2 WK). Dose administrations are indicated according to the day the letter of the chimeric poliovirus (L) and ICI (P, N) falls on. Cycle day is determined by the day it occurs within specific phases (e.g., Induction Cycle 1, Induction Cycle 2, Maintenance Cycle 1, etc.). The Maintenance Phase comprises a Maintenance Cycle that may be repeated one or more times. A total of 13 weeks are shown (see, e.g., Example 2).

FIG. 2B is a table showing a 3-week and a 6-week cycling dose schedule. A 21-day Induction Cycle comprises administering a chimeric poliovirus (e.g., lerapolturev, L) is administered weekly and a checkpoint inhibitor (ICI) is administered every 3 weeks (3 WK) or every 6 weeks (6 WK). The Induction Cycle is repeated twice which comprises the Induction Phase. Following the cessation of the Induction Phase, the Maintenance Phase begins, wherein lerapolturev and an ICI are both administered on day 1 of the 21-day Maintenance Cycle, repeated one or more times.

FIG. 2C is a table showing a 2- or 4-week cycling dose schedule. The cycle dose schedule begins with an Induction Phase comprising two consecutive 28-day Induction Cycles. The 28-day Induction Cycle 1 comprises administering a chimeric poliovirus (e.g., lerapolturev, L) weekly together with a checkpoint inhibitor (ICI) administered every 2 weeks (2 WK) or every 4 weeks (4 WK). The 28-day Induction Cycle 2 comprises administering a chimeric poliovirus (e.g., lerapolturev, L) weekly with a checkpoint inhibitor (ICI) cycling every 2 weeks (2 WK) or every 4 weeks (4 WK).

FIG. 2D is a table showing a chimeric poliovirus and ICI dose/frequency schedule with specific examples. The schedule begins with an Induction period in which a chimeric poliovirus (e.g., lerapolturev, L) may be administered weekly together with either Pembrolizumab (P) cycling every 3 weeks (3 WK) at 200 milligrams (mg) dose or every 6 weeks (6 WK) at 400 mg; or Nivolumab (N) cycling every 2 weeks (2 WK) at 240 mg or every 3 weeks (3 WK) at 360 mg or every 4 weeks (4 WK) at 480 mg. Following the cessation of the Induction Phase of administering a chimeric poliovirus (e.g., lerapolturev), a Maintenance Phase begins, wherein a chimeric poliovirus may be administered every 2 weeks (2 WK LERAPOLTUREV) or every 3 weeks (3 WK LERAPOLTUREV) or every 4 weeks (4 WK LERAPOLTUREV) or every 6 weeks (6 WK LERAPOLTUREV). For lerapolturev cycling every 3 weeks or every 6 weeks, administration will be paired with administration of pembrolizumab every 3 weeks at 200 mg or pembrolizumab every 6 weeks at 400 mg or nivolumab every 3 weeks at 360 mg. For lerapolturev cycling every 2 weeks or every 4 weeks, administration will be paired with administration of nivolumab every 4 weeks at 480 mg or nivolumab every 2 weeks at 240 mg.

FIG. 3 shows a clinical trial schematic. In the initial Screening portion of the trial participants will initially receive a booster poliovirus vaccine 1-6 weeks before the initial dose of lerapolturev. Patients then will be randomized and partitioned to two arms, including: Arm 1 (lerapolturev ONLY), and Arm 2 (lerapolturev+PD-1 inhibitor therapy). Both arms will comprise 7 weekly lerapolturev injections of up to 1.6×10⁹ TCID₅₀ injections in up to 6 lesions to initiate the dose regimen. In Arm 2, participants will be administered either pembrolizumab or nivolumab at dosage and frequency advised by the package insert. Following this, subsequent lerapolturev administrations will occur concomitantly with the timing of PD-1 inhibitor dosing (as advised by the package inserts of pembrolizumab or nivolumab). Treatment will continue until disease progression, unacceptable toxicity, or withdrawal of consent. In Arm 1, subsequent lerapolturev administrations will occur every 3 weeks. Treatment will continue until disease progression, unacceptable toxicity, or withdrawal of consent. A segment of Arm 1 may be eligible for crossover, depending on whether a participant experiences: radiologic disease progression per RECIST 1.1, no progression or confirmed partial response (PR) per RECIST 1.1 by week 26 of study, and/or a confirmed PR greater than or equal to 6 months in duration.

FIGS. 4A-4D are a collection of images of a patient with BRAF mutant (BRAF+) melanoma showing lesion changes following increased lerapolturev dose (1.6×10⁹ TCID₅₀ per administration) in combination with anti-PD-1 antibody. This participant was randomized to lerapolturev monotherapy and crossed over to lerapolturev (increased dose) in combination with anti-PD-1 after 3.5 months on study.

FIG. 4A is an image scan at baseline of a right neck BRAF+ melanoma lesion to be injected with lerapolturev.

FIG. 4B is an image scan of the right neck lesion at 1-month of lerapolturev administration.

FIG. 4C is an image scan of the right neck lesion at 5.6 months of lerapolturev administration. The right neck lesion was not palpable at this time point.

FIG. 4D is an image scan of the right neck lesion at 10.5 months of lerapolturev administration.

FIG. 4E is a plot showing lesion size changes over time. Lesion diameter (millimeters, mm) is represented on the y-axis and change in diameter for the injected right neck lesion (triangle) and the non-injected right lung lesion (circle) over time is represented by weeks since the first lerapolturev injection on the x-axis. A straight vertical line indicates the first injection of lerapolturev (week 0), and a dashed vertical line indicates the first combination dose of lerapolturev and pembrolizumab (week 14).

FIGS. 5A-5G are a collection of images showing a complete response in lesions of a patient with BRAF mutant (BRAF+) melanoma during increased lerapolturev dosing.

FIG. 5A is an image showing 4 of 5 lesions on the lower left extremity of the patient (B, bystander lesion; 1, left distal tibial lesion; 2, proximal left tibial lesion; 3, distal left tibial lesion).

FIG. 5B is an image showing the “1” left distal tibial lesion at baseline (pre-treatment) and the “1” left distal tibial lesion at 1.7-months of lerapolturev administration.

FIG. 5C is an image showing the “2” proximal left tibial lesion at baseline (pre-treatment) and the “2” proximal left tibial lesion at 1.7-months of lerapolturev administration.

FIG. 5D is an image showing the “3” distal left tibial lesion at baseline (pre-treatment) and the “3” distal left tibial lesion at 1.7-months of lerapolturev administration.

FIG. 5E is an image showing the “B” bystander lesion at baseline (pre-treatment) and the “B” bystander lesion at 1.7-months of lerapolturev administration.

FIG. 5F is an image showing the “2” proximal left tibial lesion and “3” distal left tibial lesion at 5.6-months of lerapolturev administration. Biopsies of the remaining pigmented areas were negative for melanoma.

FIG. 5G is a plot showing lesion size changes over the course of lerapolturev administration. The y-axis represents lesion diameter (millimeters, mm) changes over the weeks following first lerapolturev injection on the x-axis. Of the 9 injections of lerapolturev received, 4 were monotherapy doses under the new amendment (2.5-fold more concentrated (1.6×10⁹ TCID₅₀ per administration) than previous lerapolturev doses). Left lateral lesion, triangle; left proximal tibial lesion, large circle; left distal tibial lesion, diamond; left hamstring lesion, square; left medial by scar non-injected lesion, small circle.

FIGS. 6A-6C show an abscopal response in lesions of a patient with NRAS mutant (NRAS+) melanoma under an increased lerapolturev dosage regime (1.6×10⁹ TCID₅₀ per administration).

FIG. 6A is a PET scan image prior to the administration of lerapolturev. The patient had 4 total lesions with no visceral disease prior to beginning lerapolturev treatment. Injected lesion (arrow); uninjected lesion 1 (square); uninjected lesion 2 (circle); subcarinal lymph node lesion (triangle).

FIG. 6B is a PET scan image at 2-months of lerapolturev administration. The patient received a maximum allowable 1.6×10⁹ TCID₅₀ (3.0 ml) dose of lerapolturev throughout induction. The injected lesion (arrow) became edematous with a decrease in metabolically active cells in the center of the lesion. The uninjected lesion 1 (square) and lesion 2 (circle) decreased in size and showed decreased metabolic activity. The subcarinal lymph node lesion (triangle) appeared larger.

FIG. 6C is a plot showing lesion diameter (millimeters, mm) represented on the y-axis and change in diameter over time is represented by weeks since the first lerapolturev injection on the x-axis. A dashed line indicates the start of the first lerapolturev injection in combination with anti-PD-1 administration on cycle 1 day 1 (C1D1). Of 3 targeted lesions, 2 (non-injected) lesions (LN in right axilla, triangle; LN posterior to left scapula, circle) regressed by approximately 50% at first scan at 6 weeks following the first lerapolturev injection.

FIGS. 7A-7E show viral replication in samples isolated from individual patients.

FIG. 7A is a plot showing changes of the percent of cells exhibiting lerapolturev viral replication on the y-axis over time expressed as days on the x-axis.

FIG. 7B is a plot showing changes of the percent of CD3+CD8+ Cytotoxic T cells exhibiting lerapolturev viral replication on the y-axis over time expressed as days on the x-axis.

FIGS. 7C-E are split channel images from a single multiplexed immunofluorescence micrograph of a tumor lesion in a patient having unresectable anti-PD-1 melanoma.

FIG. 7C is a micrograph showing immunofluorescence staining of CD3 (a marker of T cells) in the infected lesion at Crossover Day 10.

FIG. 7D is a micrograph showing immunofluorescence staining of CD68 (a marker of macrophages) in the infected lesion at Crossover Day 10.

FIG. 7E is a micrograph showing immunofluorescence staining of the minus strand of lerapolturev (a marker of viral replication) in the infected lesion at Crossover Day 10.

FIG. 8 is an image reviewing key mechanisms of action for lerapolturev. Lerapolturev (formerly PVSRIPO) is a modified poliovirus-based therapy designed to safely activate the immune system to treat cancer. Lerapolturev binds to CD155 (poliovirus receptor), a cell surface TIGIT ligand expressed on a variety of solid tumors, as well as antigen presenting cells, including dendritic cells and macrophages. Lerapolturev contributes directly and indirectly in the activation of the immune system to attack the tumor microenvironment via mechanisms which include but are not limited to: (1) infecting, replicating within, then lysing cancer cells, the remnants of which become antigen sources for antigen presenting cells; (2) activating antigen presenting cells leading to T-cell priming and anticancer immunity, contributing to a systemic immune effect; and, (3) recalling pre-existing polio vaccine-specific T cell amplification of the immune response.

### DETAILED DESCRIPTION OF THE INVENTION

The present invention provides methods for treating a human patient having a cancer or which are unresponsive to previous ICI therapy. For example, multiple administrations of a chimeric poliovirus in combination with ICIs can be administered intratumorally or at another suitable delivery area to a patient having a cancer and/or one or more disease or disorders associated with tumors or which are unresponsive to previous ICI therapy.

### Terminology

Unless defined otherwise, all technical and scientific terms used herein have the same meaning as is commonly understood by one of skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Each of the references cited herein are incorporated by reference in its entirety.

The terms “a” and “an” do not denote a limitation of quantity, but rather denote the presence of at least one of the referenced items. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

As used herein, the term “about” means ±10%.

The “patient” or “subject” or “participant” treated is typically a human patient, although it is to be understood the methods described herein are effective with respect to other animals, such as mammals. More particularly, the term patient can include animals used in assays such as those used in preclinical testing including but not limited to mice, rats, monkeys, dogs, pigs, and rabbits; as well as domesticated swine (pigs and hogs), ruminants, equine, poultry, felines, bovines, murines, canines, and the like.

As used herein, the term “immune checkpoint inhibitor (ICI)” refers to therapy targeting immune checkpoint proteins, key regulators of the immune system that when expressed can dampen the immune response to an immunologic stimulus. Some cancers express ligands for the checkpoint inhibitors and can protect themselves from attack by binding to immune checkpoint targets. ICIs block inhibitory checkpoints, restoring immune system function. ICIs include those targeting immune checkpoint proteins such as PD-1, PD-1 Ligand-1 (PD-L1), PD-1 Ligand-2 (PD-L2), CTLA-4, LAG-3, TIM-3, and V-domain Ig suppressor of T-cell activation (VISTA), B7-H3/CD276, indoleamine 2,3-dioxygenase (IDO), killer immunoglobulin-like receptors (KIRs), carcinoembryonic antigen cell adhesion molecules (CEACAM) such as CEACAM-1, CEACAM-3, and CEACAM-5, sialic acid-binding immunoglobulin-like lectin 15 (Siglec-15), T cell immunoreceptor with Ig and ITIM domains (TIGIT), and B and T lymphocyte attenuator (BTLA) protein. Immune checkpoint inhibitors are known in the art.

An “effective amount” as used herein, means an amount which provides a therapeutic or prophylactic benefit.

To “treat” a disease as the term is used herein, means to reduce the frequency or severity of at least one sign or symptom of a disease, disorder, or side-effect experienced by a patient (i.e., palliative treatment) or to decrease a cause or effect of the disease, disorder (i.e., disease-modifying treatment), or side effect experienced by a patient as a result of the administration of a therapeutic agent.

As used herein, the term “response evaluation criteria in solid tumors version 1.1 (RECIST 1.1)” refers to a revised guideline that describes a standard approach to solid tumor measurements and definitions for objective change in tumor size for use in trials in which an immunotherapy is used (Eisenhauer et al. Eur J Cancer. 45:228-47(2009)).

As used herein, the term “iRECIST” refers to a consensus guideline that describes a standard approach to solid tumor measurements and definitions for objective change in tumor size for use in trials in which an immunotherapy is used (Seymour et al. Lancet Oncol. 18(3):30074-8(2019)).

As used herein, the term “complete response (CR)” refers to the disappearance of all target lesions per RECIST 1.1.

As used herein, the term “partial response (PR)” refers to greater than or equal to 30% decrease in the sum of the longest diameters of target lesions compared with baseline per RECIST 1.1.

As used herein, the term “progressive disease (PD)” refers to a 5-mm absolute increase of the sum of the longest diameters of the target lesions in addition to greater than or equal to 20% increase in the sum of the longest diameter of target lesions compared with the smallest-sum longest diameter recorded or the appearance of one or more new lesions per RECIST 1.1.

As used herein, the term “stable disease (SD)” refers to neither PR or PD occurring when evaluating target lesions per RECIST 1.1.

As used herein, the term “overall survival (OS)” refers to the time from treatment group assignment until death from any cause.

As used herein, the term “duration of response (DOR)” refers to time from confirmed objective response (CR or PR per RECIST 1.1) until unequivocal disease progression or death, whichever occurs first.

As used herein, the term “disease control rate (DCR)” refers to the proportion of patients achieving confirmed CR, confirmed PR, or SD per RECIST 1.1 as best response.

As used herein, the term “disease control rate-6months (DCR-6mo)” refers to the proportion of patients achieving confirmed CR (for any duration), confirmed PR (for any duration), or SD (greater than or equal to 6 months) per RECIST 1.1 as best response.

As used herein, the term “durable response rate” refers to the proportion of patients with confirmed CR or PR (per RECIST 1.1) last at least 6 months.

As used herein, the term “progression-free survival (PFS)” refers to the time (i.e., number of months) from treatment group assignment until date of documented radiologic disease progression per RECIST 1.1 or death due to any cause, whichever comes first.

The terms “percent identical,” “percent homologous,” or “percent similarity”, and the like, when used in the context of nucleic acid sequences refers to the residues in the two sequences being compared which are the same when aligned for maximum correspondence. The length of sequence identity comparison may be over the full-length of the sequence, or, or alternatively a fragment of at least about 50 to 2500 nucleotides. Similarly, the terms “percent identical,” “percent homologous,” or “percent similarity”, may be readily determined for amino acid sequences, over the full-length of a protein, or a fragment thereof. Suitably, a fragment is at least about 8 amino acids in length and may be up to about 7500 amino acids. Examples of suitable fragments are described herein. Generally, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments can be performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

For live viral products, such as PVSRIPO, dosage is typically expressed in plaque forming units or TCID₅₀. As used herein TCID₅₀ refers to 50% Tissue Culture Infectious Dose. Assays for determining TCID₅₀ are well known (see, e.g., Souf, S. Recent advances in diagnostic testing for viral infections. Biosci. Horizons Int. J. Student Res. 9, (2016); Pellet, E. P. et al. Basics of virology. Handb. Clin. Neurol. 123, 45-66 (2014); Gelderblom, H. R. Structure and Classification of Viruses. Medical Microbiology. 4^{th} edition, chapter 41, (1996); Reed, L. J.; Muench, H. A Simple Method of Estimating Fifty Per Cent Endpoints. Am. J. Epidemiol. 27, 493-497 (1938); Lei, C. et al. On the Calculation of TCID₅₀ for Quantitation of Virus Infectivity. Virol. Sin. 36(1), 141-144 (2021)). The TCID₅₀ is analogous (and often quantitatively similar) to the plaque-forming units (PFU) assay. A particular assay suitable for determining TCID₅₀ herein is described in U.S. Pat. No. 10,954,492, incorporated by reference herein. Another suitable assay for determining TCID₅₀ herein is described in the NIH Biopharmaceutical Development Program (BDP) Standard Operating Procedure (SOP) 22165 TCID₅₀ Assay for Poliovirus using Hep-2C Cells.

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Immune Checkpoint Inhibitors

The methods described herein include the administration of one or more ICIs. Suitable ICIs for use in the methods described herein include, but are not limited to, a programmed cell death-1 (PD-1) inhibitor, a programmed cell death-ligand (PD-L1) inhibitor, a cytotoxic T-lymphocyte-associated protein 4 (CTLA-4) inhibitor, a lymphocyte-activation gene 3 (LAG-3) inhibitor, a T-cell immunoglobulin mucin-3 (TIM-3) inhibitor, or a T cell immunoreceptor with Ig and ITIM domains (TIGIT) inhibitor, a programmed death-ligand 2 (PD-L2) inhibitor, a V-domain Ig suppressor of T-cell activation (VISTA) inhibitor, a B7-H3/CD276 inhibitor, an indoleamine 2,3-dioxygenase (IDO) inhibitor, a killer immunoglobulin-like receptor (KIR) inhibitor, a carcinoembryonic antigen cell adhesion molecule (CEACAM) against molecules such as CEACAM-1, CEACAM-3, and CEACAM-5, a sialic acid-binding immunoglobulin-like lectin 15 (Siglec-15) inhibitor, a CD47 inhibitor, a CD39 inhibitor, or a B and T lymphocyte attenuator (BTLA) protein inhibitor, or a combination thereof.

### PD-1 Inhibitors

In some embodiments, the administered immune checkpoint inhibitor is a PD-1 inhibitor that blocks the interaction of PD-1 and PD-L1 by binding to the PD-1 receptor, and in turn inhibits immune suppression. In some embodiments, the immune checkpoint inhibitor is a PD-1 immune checkpoint inhibitor selected from nivolumab (Opdivo®), pembrolizumab (Keytruda®), pidilizumab (Medivation), AMP-224 (Amplimmune); sasanlimab (PF-06801591; Pfizer), spartalizumab (PDR001; Novartis), cemiplimab (Libtayo®; REGN2810; Regeneron), retifanlimab (MGA012; MacroGenics), tislelizumab (BGB-A317; BeiGene), camrelizumab (SHR-1210; Jiangsu Hengrui Medicine Company and Incyte Corporation), CS1003 (Cstone Pharmaceuticals), and dostarlimab (TSR-042; Tesaro).

In some embodiments, the PD-1 inhibitor is nivolumab (Opdivo®) administered in an effective amount. In some embodiments, nivolumab is administered at 240 mg every 2 weeks or 480 mg every 4 weeks. In some embodiments, the PD-1 inhibitor is pembrolizumab (Keytruda®) administered in an effective amount. In some embodiments, pembrolizumab is administered at 200 mg every 3 weeks or 400 mg every 6 weeks. In some embodiments, the PD-1 inhibitor is cemiplimab (Libtayo®) administered in an effective amount. In some embodiments, cemiplimab is administered at 350 mg as an intravenous infusion over 30 minutes every 3 weeks.

### PD-L1 Inhibitors

In some embodiments, the immune checkpoint inhibitor is a PD-L1 inhibitor that blocks the interaction of PD-1 and PD-L1 by binding to the PD-L1 receptor, and in turn inhibits immune suppression. PD-L1 inhibitors include, atezolizumab (Tecentriq®, Genentech), durvalumab (Imfinzi®, AstraZeneca); avelumab (Bavencio®; Merck), envafolimab (KN035; Alphamab), BMS-936559 (Bristol-Myers Squibb), lodapolimab (LY3300054; Eli Lilly), cosibelimab (CK-301; Checkpoint Therapeutics), sugemalimab (CS-1001; Cstone Pharmaceuticals), adebrelimab (SHR-1316; Jiangsu HengRui Medicine), CBT-502 (CBT Pharma), and BGB-A333 (BeiGene).

In some embodiments, the immune checkpoint inhibitor is the PD-L1 immune checkpoint inhibitor atezolizumab (Tecentriq®) administered in an effective amount. In some embodiments, atezolizumab is administered at 840 mg every 2 weeks, 1200 mg every 3 weeks, or 1680 mg every 4 weeks. In some embodiments, atezolizumab is administered prior to chemotherapy. In another aspect of this embodiment, the immune checkpoint inhibitor is durvalumab (Imfinzi®) administered in an effective amount. In some embodiments, durvalumab is administered at 10 mg/kg every 2 weeks or 1500 mg every 4 weeks for patients that weigh more than 30 kg and 10 mg/kg every 2 weeks for patients who weigh less than 30 kg. In another aspect of this embodiment, the immune checkpoint inhibitor is avelumab (Bavencio®) administered in an effective amount. In some embodiments, avelumab is administered at 800 mg every 2 weeks. In yet another aspect of the embodiment, the immune checkpoint inhibitor is KN035 (Alphamab) administered in an effective amount. An additional example of a PD-L1 immune checkpoint inhibitor is BMS-936559 (Bristol-Myers Squibb).

### T Cell Immunoreceptor With Immunoglobulin and ITIM Domain (TIGIT) Inhibitors

In some embodiments, the immune checkpoint inhibitor is a T cell immunoreceptor with immunoglobulin and ITIM domain (TIGIT) inhibitor. TIGIT is a promising new target for cancer immunotherapy. TIGIT is upregulated by immune cells, including activated T cells, natural killer cells, and regulatory T cells. TIGIT binds to two ligands, CD155 (PVR) and CD112 (PVRL2, nectin-2), that are expressed by tumor cells and antigen-presenting cells in the tumor microenvironment (Stanietsky et al., The interaction of TIGIT with PVR and PVRL2 inhibits human NK cell cytotoxicity. Proc Natl Acad Sci USA 2009; 106: 17858-63).

TIGIT (also called WUCAM, Vstm3, VSIG9) is a receptor of the Ig superfamily, which plays a critical role in limiting adaptive and innate immunity (Boles et al., A novel molecular interaction for the adhesion of follicular CD4 T cells to follicular DC. Eur J Immunol 2009; 39:695-703). TIGIT participates in a complex regulatory network involving multiple inhibitory receptors (e.g., CD96/TACTILE, CD112R/PVRIG), one competing costimulatory receptor (DNAM-1/CD226), and multiple ligands (e.g., CD155 (PVR/NECL-5), CD112 (Nectin-2/PVRL2) (Levin et al., Vstm3 is a member of the CD28 family and an important modulator of T-cell function. Eur J Immunol 2011; 41: 902-15; Bottino et al., Identification of PVR (CD155) and nectin-2 (CD112) as cell surface ligands for the human DNAM-1 (CD226) activating molecule. J Exp Med 2003; 198: 557-67; Seth et al., The murine pan T cell marker CD96 is an adhesion receptor for CD155 and nectin-1. Biochem Biophys Res Commun 2007; 364: 959-65; Zhu et al., Identification of CD112R as a novel checkpoint for human T cells. J Exp Med 2016; 213: 167-76).

TIGIT is expressed by activated CD8+ T and CD4+ T cells, natural killer (NK) cells, regulatory T cells (Tregs), and follicular T helper cells in humans (Joller et al., Cutting edge: TIGIT has T cell-intrinsic inhibitory functions. J Immunol 2011; 186: 1338-42; Wu et al., Follicular regulatory T cells repress cytokine production by follicular helper T cells and optimize IgG responses in mice. Eur J Immunol 2016; 46: 1152-61). In sharp contrast with DNAM-1/CD226, TIGIT is weakly expressed by naive T cells. In cancer, TIGIT is co-expressed with PD-1 on tumor antigen-specific CD8+ T cells and CD8+ tumor-infiltrating lymphocytes (TILs) in mice and humans (Chauvin et al., Tigit and PD-1 impair tumor antigen-specific CD8⁺ T cells in melanoma patients. J Clin Invest 2015; 125: 2046-58; Johnston et al., The immunoreceptor TIGIT regulates antitumor and antiviral CD8(+) T cell effector function. Cancer Cell 2014; 26: 923-37). It is also co-expressed with other inhibitory receptors, such as T cell immunoglobulin and mucin domain-containing molecule-3 (TIM-3) and lymphocyte activation gene 3 (LAG-3), on exhausted CD8+ T cell subsets in tumors (Chauvin et al., Tigit and PD-1 impair tumor antigen-specific CD8⁺ T cells in melanoma patients. J Clin Invest 2015; 125: 2046-58; Johnston et al., The immunoreceptor TIGIT regulates antitumor and antiviral CD8(+) T cell effector function. Cancer Cell 2014; 26: 923-37). Further, TIGIT is highly expressed by Tregs in peripheral blood mononuclear cells of healthy donors and patients with cancer and further upregulated in the TME (Joller et al., Treg cells expressing the coinhibitory molecule TIGIT selectively inhibit proinflammatory Th1 and Th17 cell responses. Immunity 2014; 40: 569-81; Zhang et al., Genome-Wide DNA methylation analysis identifies hypomethylated genes regulated by FOXP3 in human regulatory T cells. Blood 2013; 122: 2823-36).

In some embodiments, the immune checkpoint inhibitor is a TIGIT inhibitor that blocks the interaction of TIGIT and CD155 by binding to the TIGIT receptor, and in turn inhibits immune suppression. TIGIT inhibitors include, but are not limited to, Etigilimab (OMP-313M32; Oncomed Pharmaceuticals); Tiragolumab (MTIG7192A; RG6058; Roche/Genentech); Vibostolimab (MK-7684; Merck); BMS-986207 (Bristol-Myers Squibb); AZD2936 (AstraZeneca); ASP8374 (Astellas/Potenza Therapeutics); Domvanalimab (AB154; Arcus Biosciences); IBI939 (Innovent Biologics); Ociperlimab (BGB-A1217; BeiGene); EOS884448 (iTeos Therapeutics); SEA-TGT (Seattle Genetics); COM902 (Compugen); MPH-313 (Mereo Biopharma); M6223 (EMD Serono); HLX53 (Shanghai Henlius Biotech); JS006 (Junshi Bio); mAb-7 (Stanwei Biotech); SHR-1708 (Hengrui Medicine); BAT6005 (Bio-Thera Solutions); GS02 (Suzhou Zelgen/Qilu Pharma); RXI-804 (Rxi Pharmaceuticals); NB6253 (Northern Biologics); ENUM009 (Enumreal Biomedical); CASC-674 (Cascadian Therapeutics); AJUD008 (AJUD Biopharma); and AGEN1777 (Agenus, Bristol-Myers Squibb)).

### T-Cell Immunoglobulin and Mucin Domain 3 (TIM-3) Inhibitors

In some embodiments, the immune checkpoint inhibitor is a T-cell immunoglobulin and mucin domain 3 (TIM-3) inhibitor. TIM-3 is an immunoglobulin (Ig) and mucin domain-containing cell surface molecule that was originally discovered as a cell surface marker specific to interferon (IFN-γ) producing CD4⁺ T helper 1 (Th1) and CD8⁺ T cytotoxic 1 (Tc1) cells (Monney et al., Th1-specific cell surface protein Tim-3 regulates macrophage activation and severity of an autoimmune disease. Nature 2002; 415: 536-41). Tim-3 is coregulated and co-expressed along with other immune checkpoint receptors (PD-1, Lag-3, and TIGIT) on CD4⁺ and CD8⁺ T cells (Chihara et al., Induction and transcriptional regulation of the co-inhibitory gene module in T cells. Nature 2018; 558: 454-9; DeLong et al., Il-27 and TCR stimulation promote T cell expression of multiple inhibitory receptors. ImmunoHorizons 2019; 3: 13-25). In cancer, Tim-3 expression specifically marks the most dysfunctional or terminally exhausted subset of CD8⁺ T cells (Fourcade et al., Upregulation of Tim-3 and PD-1 expression is associated with tumor antigen-specific CD8+ T cell dysfunction in melanoma patients. J Exp Med 2010; 207: 2175-86; Sakuishi et al., Targeting Tim-3 and PD-1 pathways to reverse T cell exhaustion and restore anti-tumor immunity. J Exp Med 2010; 207: 2187-94). Four ligands for Tim-3 have been identified: galectin-9, phosphatidylserine (PtdSer), high-mobility group protein B1 (HMGB1), and CEACAM-1.

In some embodiments, the ICI is a TIM-3 inhibitor that blocks the interaction of TIM-3 and galectin-9, phosphatidylserine (PtdSer), high-mobility group protein B1 (HMGB1), and/or CEACAM-1 by binding to the TIM-3 receptor, and in turn inhibits immune suppression. TIM-3 inhibitors include, but are not limited to, Sabatolimab (MGB453; Novartis Pharmaceuticals); Cobolimab (TSR-022; Tesaro/GSK); RG7769 (Genentech); MAS-825 (Novartis); Sym023 (Symphogen A/S); BGBA425 (BeiGene); R07121661 (Hoffmann-La Roche); LY3321367 (Eli Lilly and Company); INCAGN02390 (Incyte Corporation); BMS-986258 (ONO7807, Bristol-Myers Squibb); AZD7789 (AstraZeneca); TQB2618 (Chia Tai Tianqing Pharmaceutical Group Co., Ltd.); and NB002 (Neologics Bioscience).

### Lymphocyte Activation Gene-3 (LAG-3) Inhibitors

In some embodiments, the immune checkpoint inhibitor is a LAG-3 inhibitor. LAG-3 (CD223) is encoded by the LAG-3 gene. LAG-3 is a member of the immunoglobulin superfamily (IgSF) and exerts a wide variety of biologic impacts on T cell function (Triebel et al., LAG-3, a novel lymphocyte activation gene closely related to CD4. J Exp Med 1990; 171: 1393-405). LAG-3 is expressed on cell membranes of natural killer cells (NK), B cells, tumor-infiltrating lymphocytes (TIL), a subset of T cells, and dendritic cells (DC) (Triebel et al., LAG-3, a novel lymphocyte activation gene closely related to CD4. J Exp Med 1990; 171: 1393-405); Kisielow et al., Expression of lymphocyte activation gene 3 (LAG-3) on B cells is induced by T cells. Eur J Immunol 2005; 35: 2081-8; Grosso et al., LAG-3 regulates CD8+ T cell accumulation and effector function in murine self- and tumor-tolerance systems. J Clin Invest 2007; 117: 3383-92; Workman et al., LAG-3 regulates plasmacytoid dendritic cell homeostasis. J Immunol 2009; 182: 1885-91; Andreae et al., Maturation and activation of dendritic cells induced by lymphocyte activation gene-3 (CD223). J Immunol 2002; 168: 3874-80). The LAG-3 protein binds a nonholomorphic region of major histocompatibility complex 2 (MHC class II) with greater affinity than CD 4 (Baixeras et al., Characterization of the lymphocyte activation gene 3-encoded protein. A new ligand for human leukocyte antigen class II antigens. J Exp Med 1992; 176: 327-37). LAG-3 is one of the various immune-checkpoint receptors that are coordinately upregulated on both regulatory T cells (Tregs) and anergic T cells, and the simultaneous blockade of these receptors can result in an enhanced reversal of this anergic state relative to the blockade of one receptor alone (Grosso et al., Functionally distinct LAG-3 and PD-1 subsets on activated and chronically stimulated CD8 T cells. J Immunol 2009; 182: 6659-69). The LAG-3/MHC class II molecule interaction leads to the downregulation of CD4+ Ag-specific T cell clone proliferation and cytokine secretion (Huard et al., T cell major histocompatibility complex class II molecules down-regulate CD4+ T cell clone responses following LAG-3 binding. Eur J Immunol 1996; 26: 1180-6).

In some embodiments, the checkpoint inhibitor is a LAG-3 inhibitor that blocks the interaction of LAG-3 with major histocompatibility complex 2 (MHC class II) by binding to the LAG-3 receptor, and in turn inhibits immune suppression. LAG-3 inhibitors include, but are not limited to, relatlimab (BMS 986016/Ono 4482; Bristol-Myers Squibb); tebotelimab (MGD013; Macrogenics); LAG525 (Immutep, Novartis); TSR-033 (Tesaro, GlaxoSmithKline); Eftilagimod alpha (IMP321, Immutep); REGN3767 (Regeneron); INCAGN02385 (Incyte); RO7247669 (Hoffman-LaRoche); Favezelimab (Merck Sharp & Dohme); CB213 (Crescendo Biologics); FS118 (F-star Therapeutics); SYM022 (Symphogen); GSK2831781 (GlaxoSmithKline); IBI323 (Innovent Biologics (Suzhou) Co. Ltd.); EMB-02 (Shanghai EpimAb Biotherapeutics Co., Ltd.); SNA03 (Microbio Group); and AVA021 (Avacta).

### Additional Immune Checkpoint Inhibitors

In some embodiments, the patient is administered a B7-H3/CD276 immune checkpoint inhibitor such as enoblituzumab (MGA217, Macrogenics) MGD009 (Macrogenics), 131I-8H9/omburtamab (Y-mabs), and I-8H9/omburtamab (Y-mabs), an indoleamine 2,3-dioxygenase (IDO) ICI such as Indoximod and INCB024360, a killer immunoglobulin-like receptors (KIR) immune checkpoint inhibitor such as Lirilumab (BMS-986015), a carcinoembryonic antigen cell adhesion molecule (CEACAM) inhibitor (e.g., CEACAM-1, -3 and/or -5). Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146) or cross-reacts with CEACAM-1 and CEACAM-5 as described in, e.g., WO 2013/054331 and US 2014/0271618.

In some embodiments, the patient is administered an ICI directed to CD47, including, but not limited to, Hu5F9-G4 (Stanford University/Forty Seven), TI-061 (Arch Oncology), TTI-622 (Trillum Therapeutics), TTI-621 (Trillum Therapeutics), SRF231 (Surface Oncology), SHR-1603 (Hengrui), OSE-172 (Boehringer Ingelheim/OSE Immunotherapeutics), NI-1701 (Novimmune TG Therapeutics), IBI188 (Innovent Biologics); CC-95251 (Celgene), CC-90002 (Celgene/Inibrx), AO-176 (Arch Oncology), ALX148 (ALX Oncology), IMM01 (ImmuneOnco Biopharma), IMM2504 (ImmuneOnco Biopharma), IMM2502 (ImmuneOnco Biopharma), IMM03 (ImmuneOnco Biopharma), IMC-002 (ImmuneOncia Therapeutics), IBI322 (Innovent Biologics), HMBD-004B (Hummingbird Bioscience), HMBD-004A (Hummingbird Bioscience), HLX24 (Henlius), FSI-189 (Forty Seven), DSP107 (KAHR Medical), CTX-5861 (Compass Therapeutics), BAT6004 (Bio-Thera), AUR-105 (Aurigene), AUR-104 (Aurigene), ANTI-CD47 (Biocad), ABP-500 (Abpro), ABP-160 (Abpro), TJC4 (I-MAB Biopharma), TJC4-CK (I-MAB Biopharma), SY102 (Saiyuan), SL-172154 (Shattuck Labs), PSTx-23 (Paradigm Shift Therapeutics), PDL1/CD47BsAb (Hanmi Pharmaceuticals), NI-1801 (Novimmune), MBT-001 (Morphiex), LYN00301 (LynkCell), and BH-29xx (Beijing Hanmi).

In some embodiments, the ICI is an inhibitor directed to CD39, including, but not limited to TTX-030 (Tizona Therapeutics), IPH5201 (Innate Pharma/AstraZeneca), SRF-617 (Surface Oncology), ES002 (Elpisciences), 9-8B (Igenica), and an antisense oligonucleotide (Secarna).

In some embodiments, the immune checkpoint inhibitor is an inhibitor directed to B and T lymphocyte attenuator molecule (BTLA), for example as described in Zhang et al., Monoclonal antibodies to B and T lymphocyte attenuator (BTLA) have no effect on in vitro B cell proliferation and act to inhibit in vitro T cell proliferation when presented in a cis, but not trans, format relative to the activating stimulus, Clin Exp Immunol. 2011 January; 163(1): 77-87, and TAB004/JS004 (Junshi Biosciences).

In some embodiments, the immune checkpoint inhibitor is a sialic acid-binding immunoglobulin-like lectin 15 (Siglec-15) inhibitor, including, but not limited to, NC318 (an anti-Siglec-15 mAb).

In some embodiments, the ICI is opdualag, a combination of the LAG-3 checkpoint inhibitor relatimab and the PD-1 inhibitor nivolumab.

### Cancer or Tumor Types

As contemplated herein, the specifically-timed, frequently administered, high dose of a chimeric poliovirus in combination with an ICI can be used in the treatment of a subject having a cancer or tumor. In some embodiments, the cancer is a solid cancer or tumor. In some embodiments, the cancer or tumor is a non-solid cancer or tumor. In some embodiments, the solid tumor expresses PD-L1 or is considered a PD-L1 positive tumor. In some embodiments, the cancer is a cancer previously treated with PD-1 inhibitor and/or PD-L1 inhibitor therapy. In some embodiments, the cancer is a cancer previously treated with chemotherapy. In some embodiments, the cancer is a PD-1 refractory cancer. In some embodiments, the cancer is a PD-L1 refractory cancer. In some embodiments, the cancer is a PD-L1+cancer. In some embodiments, the cancer is an advanced metastatic cancer. In some embodiments, the cancer is an unresectable cancer. In some embodiments, the cancer is a resectable cancer. In some embodiments, the methods described herein are used as a first line therapy to treat a patient with a cancer. In some embodiments, the methods described herein are used as a second line therapy to treat a patient with a cancer. In some embodiments, the human patient is an adult with a low risk of developing cancer. In some embodiments, the human patient is an adult with a high risk of developing cancer. In some embodiments, the chimeric poliovirus administration is an intratumoral administration. In some embodiments, the chimeric poliovirus administration is an intravesical administration. In some embodiments, the chimeric poliovirus administration is a topical administration. In some embodiments, the chimeric poliovirus is administered as a neoadjuvant. In some embodiments, the chimeric poliovirus is administered as an adjuvant to a surgical sight following resection. In some embodiments, the chimeric poliovirus is administered by injection to deep visceral lesions. In some embodiments, the chimeric poliovirus is administered by convection enhanced delivery. In some embodiments, the chimeric poliovirus is administered by intracerebral infusion with convection enhanced delivery. In some embodiments, the chimeric poliovirus administration is stereotactically guided. In some embodiments, the PD-1 and or PD-L1 inhibitor(s) is administered by intravenous injection. In some embodiments, the PD-1 and or PD-L1 inhibitor(s) is administered by intratumoral injection. In some embodiments, the PD-1 and or PD-L1 inhibitor(s) is administered by subcutaneous injection. In some embodiments, the PD-1 and or PD-L1 inhibitor(s) is administered by intravesical injection.

In some embodiments, the cancer is selected from glioblastoma multiforme (GBM), astrocytoma, oligodendroglioma, astro-oligodendroglioma, renal cell carcinoma, prostate cancer, bladder cancer, esophageal cancer, stomach cancer, pancreas cancer, colorectal cancer, liver cancer, gall bladder cancer, breast cancer, medulloblastoma, lung cancer, head and neck squamous cell carcinoma (HNSCC), melanoma, ovarian cancer, or sarcoma.

In some embodiments, the methods described herein are used to treat a human patient with glioblastoma multiforme (GBM). In some embodiments, the GBM is recurrent GBM in adults. In some embodiments, the GBM is post-surgery recurrent GBM. In some embodiments, the GBM is a GBM previously treated with PD-1 inhibitor and/or PD-L1 inhibitor therapy.

In some embodiments, the methods described herein are used to treat a human patient with astrocytoma. In some embodiments, the astrocytoma is an astrocytoma previously treated with PD-1 inhibitor and/or PD-L1 inhibitor therapy.

In some embodiments, the methods described herein are used to treat a human patient with oligodendroglioma. In some embodiments, the oligodendroglioma is an oligodendroglioma previously treated with PD-1 inhibitor and/or PD-L1 inhibitor therapy.

In some embodiments, the methods described herein are used to treat a patient with astro-oligodendroglioma. In some embodiments, the astro-oligodendroglioma is an astro-oligodendroglioma previously treated with PD-1 inhibitor and/or PD-L1 inhibitor therapy.

In some embodiments, the methods described herein are used to treat a human patient with medulloblastoma. In some embodiments, the medulloblastoma is a medulloblastoma previously treated with PD-1 inhibitor and/or PD-L1 inhibitor therapy.

In some embodiments, the methods described herein are used to treat a human patient with renal cell carcinoma. In some embodiments, the renal cell carcinoma is a renal cell carcinoma previously treated with PD-1 inhibitor and/or PD-L1 inhibitor therapy.

In some embodiments, the methods described herein are used to treat a human patient with prostate cancer. In some embodiments, the prostate cancer is a prostate cancer previously treated with PD-1 inhibitor and/or PD-L1 inhibitor therapy.

In some embodiments, the methods described herein are used to treat a human patient with bladder cancer. In some embodiments, the bladder cancer is a resectable cisplatin-ineligible/refusal muscle invasive bladder cancer (MIBC). In some embodiments, the bladder cancer is a locally advanced or metastatic bladder cancer that has not progressed with first-line platinum-containing chemotherapy. In some embodiments, the bladder cancer is a Bacillus-Calmette-Guerin (BCG)-unresponsive, high-risk, non-muscle invasive bladder cancer (NMIBC) with carcinoma in situ (CIS) with or without papillary tumors who are ineligible for or have elected not to undergo cystectomy. In some embodiments, the bladder cancer is a carcinoma in situ (CIS) of the urinary bladder. In some embodiments, the bladder cancer is a primary or recurrent stage Ta and/or T1 papillary bladder cancer tumors following transurethral resection (TUR). In some embodiments, the human patient is an adult with a low risk of developing NMIBC. In some embodiments, the human patient is an adult with a high risk of developing NMIBC. In some embodiments, the bladder cancer is a bladder cancer previously treated with PD-1 inhibitor and/or PD-L1 inhibitor therapy. In some embodiments, the chimeric poliovirus administration is an intratumoral administration. In some embodiments, the chimeric poliovirus administration is an intravesical administration.

In some embodiments, the methods described herein are used to treat a human patient with esophageal cancer. In some embodiments, the esophageal cancer is a high-grade dysplasia in Barrett esophagus in patients who do not undergo esophagectomy. In some embodiments, the esophageal cancer is a low-grade dysplasia in Barrett esophagus in patients who do not undergo esophagectomy. In certain embodiments, the chimeric poliovirus administration is an intratumoral administration. In certain embodiments, the chimeric poliovirus administration is a topical administration. In some embodiments, the esophageal cancer is an esophageal cancer previously treated with PD-1 inhibitor and/or PD-L1 inhibitor therapy.

In some embodiments, the methods described herein are used to treat a human patient with stomach cancer. In some embodiments, the stomach cancer is a stomach cancer previously treated with PD-1 inhibitor and/or PD-L1 inhibitor therapy.

In some embodiments, the methods described herein are used to treat a human patient with pancreas cancer. In some embodiments, the pancreas cancer is a pancreas cancer previously treated with PD-1 inhibitor and/or PD-L1 inhibitor therapy.

In some embodiments, the methods described herein are used to treat a human patient with colorectal cancer. In some embodiments, the colorectal cancer is a colorectal cancer previously treated with fluoropyrimidine-, oxaliplatin- and irinotecan-based chemotherapy, an anti-VEGF therapy, and, if RAS wild-type, an anti-EGFR therapy. In some embodiments, the colorectal cancer is a metastatic colorectal cancer previously treated with fluoropyrimidine- and/or oxaliplatin-based chemotherapy. In some embodiments, the colorectal cancer is a colorectal cancer previously treated with PD-1 inhibitor and/or PD-L1 inhibitor therapy. In some embodiments, the colorectal cancer is a BRAF mutant colorectal cancer.

In some embodiments, the methods described herein are used to treat a human patient with liver cancer. In some embodiments, the liver cancer is a liver cancer previously treated with PD-1 inhibitor and/or PD-L1 inhibitor therapy.

In some embodiments, the methods described herein are used to treat a human patient with gall bladder cancer. In some embodiments, the gall bladder cancer is a gall bladder cancer previously treated with PD-1 inhibitor and/or PD-L1 inhibitor therapy.

In some embodiments, the methods described herein are used to treat a human patient with breast cancer. In some embodiments, the breast cancer is a triple negative breast cancer (TNBC). In some embodiments, the breast cancer is a breast cancer previously treated with PD-1 inhibitor and/or PD-L1 inhibitor therapy.

In some embodiments, the methods described herein are used to treat a human patient with medulloblastoma. In some embodiments, the GBM is a GBM previously treated with PD-1 inhibitor and/or PD-L1 inhibitor therapy.

In some embodiments, the methods described herein are used to treat a human patient with lung cancer. In some embodiments, the lung cancer is a lung cancer previously treated with PD-1 inhibitor and/or PD-L1 inhibitor therapy.

In some embodiments, the methods described herein are used to treat a patient with head and neck squamous cell carcinoma (HNSCC). In some embodiments, the HNSCC is a metastatic or unresectable, recurrent HNSCC with PD-L1+ tumors [Combined Positive Score (CPS)≥1] as determined by an FDA-approved test. In some embodiments, the HNSCC is a locally advanced resectable Stage II-Iva HNSCC. In some embodiments, the HNSCC is a HNSCC previously treated with PD-1 inhibitor and/or PD-L1 inhibitor therapy.

In some embodiments, the methods described herein are used to treat a patient with melanoma. In some embodiments, the melanoma is a PD-1 refractory melanoma. In some embodiments, the melanoma is an advanced PD-1 refractory melanoma. In some embodiments, the melanoma is a PD-L1 refractory melanoma. In some embodiments, the melanoma is PD-1/PD-L1 inhibitor refractory Stage 0-II Melanoma. In some embodiments, the melanoma is resectable Stage 0-II Melanoma. In some embodiments, the melanoma is resectable metastatic Stage III-IV Melanoma. In some embodiments, the melanoma is unresectable metastatic Stage III-IV Melanoma. In some embodiments, the melanoma is an PD-1/PD-L1 inhibitor refractory unresectable metastatic Stage III-IV Melanoma. In some embodiments, the melanoma is PD-1/PD-L1 inhibitor refractory resectable, metastatic Stage III-IV Melanoma. In some embodiments, the melanoma is refractory unresectable, metastatic Stage III-IV Melanoma. In some embodiments, the melanoma is a melanoma previously treated with PD-1 inhibitor and/or PD-L1 inhibitor therapy. In some embodiments, the melanoma is a BRAF (B-Raf Proto-Oncogene, Serine/Threonine Kinase)-mutant melanoma. In some embodiments, the melanoma is a NRAS (NRAS Proto-Oncogene, GTPase)-mutant melanoma. In some embodiments, the melanoma is a KIT-mutant melanoma. In some embodiments, the melanoma is a GNAQ (Guanine Nucleotide Binding Protein (G Protein), Q Polypeptide)-mutant melanoma. In some embodiments, the melanoma is a GNA11 (G Protein Subunit Alpha 11)-mutant melanoma. In some embodiments, the melanoma is a MEK (Mitogen-Activated Protein Kinase Kinase 1)-mutant melanoma.

In some embodiments, the methods described herein are used to treat a patient with ovarian cancer. In some embodiments, the ovarian cancer is a platinum-resistant ovarian cancer. In some embodiments, the ovarian cancer is an ovarian cancer previously treated with PD-1 inhibitor and/or PD-L1 inhibitor therapy.

In some embodiments, the methods described herein are used to treat a patient with sarcoma. In some embodiments, the sarcoma is a sarcoma previously treated with PD-1 inhibitor and/or PD-L1 inhibitor therapy.

### Improved Patient Outcomes

In some embodiments, the administration of a chimeric poliovirus and ICI treatment regimen described herein provides enhanced objective response rate (ORR) in the patients receiving the treatment. ORR is generally defined as the proportion of patients achieving a complete response (CR) or partial response (PR) per RECIST 1.1. Examples of an objective response (OR) includes a complete response (CR), which is the disappearance of all signs of the tumor in response to treatment and a partial response (PR), which is a decrease in the size of a tumor in response to treatment. In some embodiments, the OR is a CR. In some embodiments, the OR is a PR. The ORR is an important parameter to demonstrate the efficacy of a treatment and it serves as a primary or secondary endpoint in clinical trials. Methods of assessing ORR are well known in the art and include, for example RECIST v1.1 (Eisenhauer et al. Eur J Cancer. 45:228-47(2009)) and World Health Organization (WHO) (World Health Organization. WHO Handbook for Reporting Results of Cancer Treatment. World Health Organization Offset Publication No. 48; Geneva (Switzerland), 1979).

In some embodiments, the administration of a chimeric poliovirus and ICI treatment regimen described herein provides a greater proportion of patients achieving confirmed CR according to RECIST 1.1 criteria. In some embodiments, the administration of a chimeric poliovirus and ICI combination treatment regimen described herein provides a greater proportion of patients achieving confirmed PR according to RECIST 1.1 criteria. In some embodiments, the administration of a chimeric poliovirus and ICI combination treatment regimen described herein provides a significant change from baseline in the number of CD8+tumor infiltrating lymphocytes (TILs) in the tumor microenvironment (TME) of injected and non-injected lesions. In some embodiments, the administration of a chimeric poliovirus and ICI combination treatment regimen described herein provides a significant increase from baseline in PD-L1 expression in cells in the

TME of injected and non-injected lesions. In some embodiments, the administration of a chimeric poliovirus and ICI combination treatment regimen described herein provides extended overall survival (OS). Overall survival (OS) is generally defined as the time from treatment group assignment until death from any cause. In some embodiments, the administration of a chimeric poliovirus and ICI combination treatment regimen described provides improved duration of response (DOR). Duration of response (DOR) is generally defined as the time from OR per RECIST 1.1 until unequivocal disease progression or death, whichever occurs first.

In some embodiments, the administration of a chimeric poliovirus and ICI combination treatment regimen described herein provides improved disease control rate (DCR). Disease control rate (DCR) is generally defined as the proportion of patients achieving CR, PR, or stable disease (SD) per RECIST 1.1, as best response. In some embodiments, the administration of a chimeric poliovirus and ICI combination treatment regimen described herein provides improved disease control rate-6months (DCR-6mo). Disease control rate-6months (DCR-6mo) is generally defined as the proportion of patients achieving CR (for any duration), PR (for any duration), or SD (greater than or equal to 6 months) per RECIST 1.1 as best response.

In some embodiments, the administration of a chimeric poliovirus and ICI combination treatment regimen described herein provides improved durable response rate (DRR). Durable response rate (DRR) is generally defined as the proportion of patients with CR or PR (per RECIST 1.1) lasting at least 6 months.

In some embodiments, the administration of a chimeric poliovirus and ICI combination treatment regimen described herein provides extended progression-free survival (PFS). Progression-free survival (PFS) is generally defined as the time (number of months) from treatment group assignment until date of documented radiologic disease progression per RECIST 1.1 or death due to any cause, whichever comes first.

In some embodiments, the administration of a chimeric poliovirus and ICI combination treatment regimen described herein provides alterations in immune markers (e.g., immune cell density, T cell receptor repertoire, chemokine profile, cytokine profile) in blood samples and/or tissue samples. In some embodiments, the administration of a chimeric poliovirus and ICI combination treatment regimen described herein provides changes in genetic, cytologic, histologic, and/or other markers in tumor biopsies and peripheral blood mononuclear cells (PBMCs) that correlate with response. In some embodiments, the administration of a chimeric poliovirus construct and ICI combination treatment regimen described herein provides improved ORR/DOR, DCR, and DCR-6-mo based on iRECIST criteria. In some embodiments, the administration of a chimeric poliovirus and ICI combination treatment regimen described herein provides altered ORR, DOR, DRR, DCR, and DCR-6-mo in the following melanoma patient subgroups: acquired versus primary PD-1/PD-L1 inhibitor resistant patients as previously defined (Kluger et al. J Immunother Cancer. 8(1):e000398(2020)), BRAF wild type and mutant patients, LDH levels at baseline patients, time since last dose of PD-1/PD-L1 inhibitor therapy prior to randomization (less than or equal to or greater than 6 weeks), and those previously treated with chimeric poliovirus monotherapy. In some embodiments, the administration of a chimeric poliovirus and ICI combination treatment regimen described herein provides increased OS and PFS in the following melanoma subgroups: according to treatment arm and AJCC stage at baseline, acquired versus primary PD-1/PD-L1 inhibitor resistant patients as previously defined (Kluger et al. J Immunother Cancer. 8(1):e000398(2020)), BRAF wild type and mutant patients, LDH levels at baseline patients, time since last dose of PD-1/PD-L1 inhibitor therapy prior to randomization (less than or equal to or greater than 6 weeks), and crossover to combination arm from chimeric poliovirus construct monotherapy.

### Pharmaceutical Compositions and Dosage Forms

The chimeric poliovirus for administration in the methods described herein can be administered, for example, as a pharmaceutical composition that includes an effective amount for a patient, typically a human, in need of such treatment in a pharmaceutically acceptable carrier.

Carriers include excipients and diluents and should be of sufficiently high purity and sufficiently low toxicity to render them suitable for administration to the patient being treated. The carrier can be inert, or it can possess pharmaceutical benefits of its own. The amount of carrier employed in conjunction with the compound is sufficient to provide a practical quantity of material for administration per unit dose of the compound.

Classes of carriers include, but are not limited to adjuvants, binders, buffering agents, coloring agents, diluents, disintegrants, excipients, emulsifiers, flavorants, gels, glidents, lubricants, preservatives, stabilizers, surfactants, solubilizer, tableting agents, wetting agents, or solidifying material.

Exemplary pharmaceutically acceptable carriers include sugars, starches, celluloses, powdered tragacanth, malt, gelatin, talc, petroleum jelly, lanoline, polyethylene glycols, alcohols, transdermal enhancers, and vegetable oils. Optional active agents may be included in a pharmaceutical composition, which do not substantially interfere with the activity of the compound of the present invention.

Some excipients include, but are not limited, to liquids such as water, saline, glycerol, polyethylene glycol, hyaluronic acid, ethanol, and the like. The compound can be provided, for example, in the form of a solid, a liquid, spray dried material, a microparticle, nanoparticle, controlled release system, etc., as desired according to the goal of the therapy. Suitable excipients for non-liquid formulations are also known to those of skill in the art. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable, and which provides the formulation with the desired pH, i.e., a pH in the physiologically acceptable range. Examples of buffer solutions include saline, phosphate buffered saline, Tris buffered saline, Hank's buffered saline, and the like.

In yet another embodiment provided is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and thiolated polymers (carboxymethyl cellulose-cysteine, polycarbophil-cysteine, chitosan-thiobutylamidine, chitosan-thioglycolic acid, chitosan-glutathione conjugates).

In certain embodiments the excipient is selected from butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Typically, sterile injectable suspensions are formulated according to techniques known in the art using suitable carriers, dispersing or wetting agents and suspending agents. The sterile injectable formulation can also be a sterile injectable solution or a suspension in an acceptably nontoxic parenterally acceptable diluent or solvent. Among the acceptable vehicles and solvents that can be employed are water, Ringer's solution, and isotonic sodium chloride solution. In addition, sterile, fixed oils, fatty esters, or polyols are conventionally employed as solvents or suspending media. In addition, parenteral administration can involve the use of a slow release or sustained release system such that a constant level of dosage is maintained.

Injectable formulations can be prepared in conventional forms, either as liquid solutions or suspensions, solid forms suitable for solubilization or suspension in liquid prior to injection, or as emulsions. Typically, sterile injectable suspensions are formulated according to techniques known in the art using suitable carriers, dispersing or wetting agents and suspending agents. The sterile injectable formulation can also be a sterile injectable solution or a suspension in an acceptably nontoxic parenterally acceptable diluent or solvent. Among the acceptable vehicles and solvents that can be employed are water, Ringer's solution, and isotonic sodium chloride solution. In addition, sterile, fixed oils, fatty esters, or polyols are conventionally employed as solvents or suspending media. In addition, parenteral administration can involve the use of a slow release or sustained release system such that a constant level of dosage is maintained.

The pharmaceutical composition comprising the chimeric poliovirus may be administered in a therapeutically effective amount by any desired mode of administration, but is typically administered as intratumoral injection or infusion, or alternatively, topically applied to a tumor lesion. Administration via intratumoral injection can involve introducing the formulations of the disclosure into one or more tumor lesions of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

In some embodiments, when the chimeric poliovirus is lerapolturev, lerapolturev is formulated in 50 mM sodium phosphate in 0.9% sodium chloride, pH 7.4 with 0.2% human serum albumin (HSA) in phosphate buffered saline (PBS). Lerapolturev can be provided in sterile, single use glass vials with a flip off top containing approximately 0.5 mL of stock lerapolturev (for example, about 2.24×10⁹ TCID₅₀).

In some embodiments, the chimeric poliovirus is lerapolturev, and is administered at a fixed concentration of between about 4.0×10⁸ TCID₅₀/ml and about 6.0×10⁸ TCID₅₀/ml, wherein a minimum of approximately 0.5 ml is administered per tumor lesion. In some embodiments, lerapolturev is administered at about 5.33×10⁸ TCID₅₀/ml. In some embodiments, the maximum volume injected at an individual treatment visit will be between about 2.5 ml and about 3.5 ml. In some embodiments, the maximum volume injected at an individual treatment visit is about 3 ml. In some embodiments, the maximum lerapolturev dose administered is between about 1.0×10⁹ TCID₅₀ to about 2.0×10⁹ TCID₅₀. In some embodiments, the maximum lerapolturev dose administered is about 1.6×10⁹ TCID₅₀. In some embodiments, the maximum lerapolturev dose administered is up to about 1.0×10¹⁰ TCID₅₀. In some embodiments, the chimeric poliovirus is lerapolturev, and is administered at a fixed concentration of between about 1.6×10⁹ TCID₅₀/ml and about 2.0×10¹⁰ TCID₅₀/ml. In some embodiments, the maximum lerapolturev dose administered is about 1.0×10¹⁰ TCID₅₀. In some embodiments, the minimum injection volume is 0.5 ml. In some embodiments, the minimum lerapolturev dose administered at an individual treatment visit is about 2.67×10⁸ TCID₅₀. As provided herein, up to 6 lesions may be injected at an individual treatment visit. In some embodiments, up to 10 lesions may be injected at an individual treatment visit. It is possible that more than 1 syringe may be required to treat a single lesion, due to large lesion size, for example as provided in Table 2 below.

### EXAMPLES

The claimed invention is further described by way of the following non-limiting examples. Further aspects and embodiments of the present invention will be apparent to those of ordinary skill in the art, in view of the above disclosure and following experimental exemplification, included by way of illustration and not limitation, and with reference to the attached figures.

### Example 1. Multiple Doses of PVSRIPO Increases Efficacy of Anti-Tumor Response in Combination With Anti-PD-1 Treatment

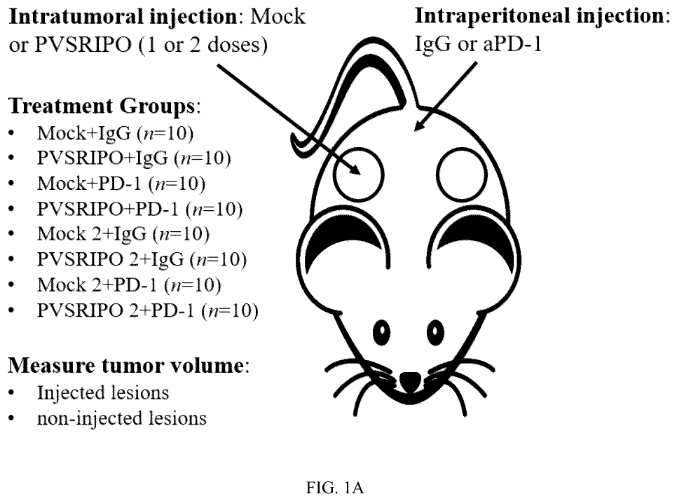

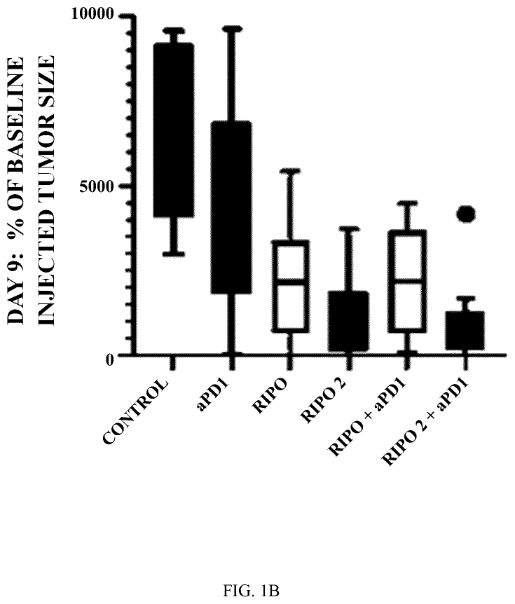

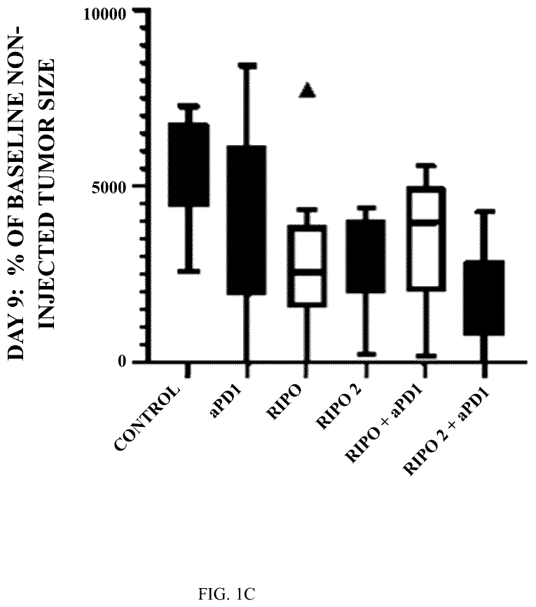

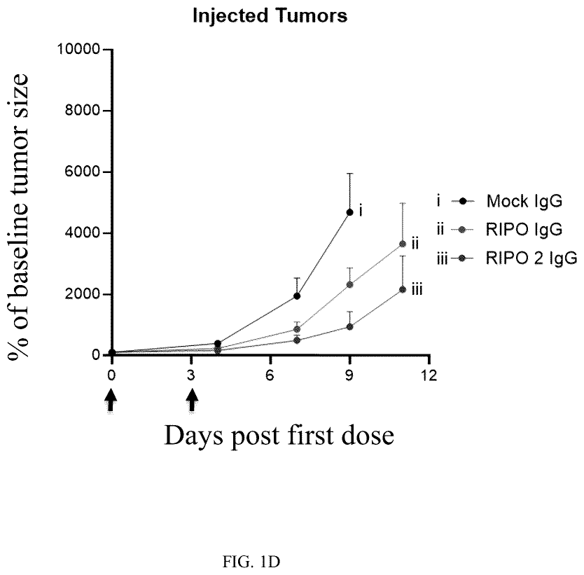

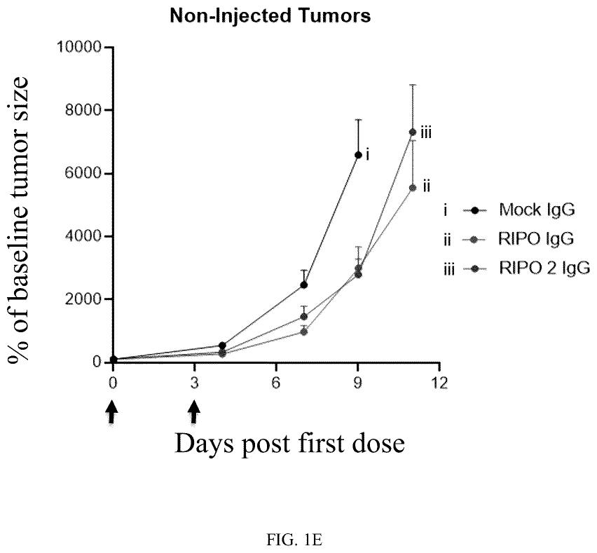

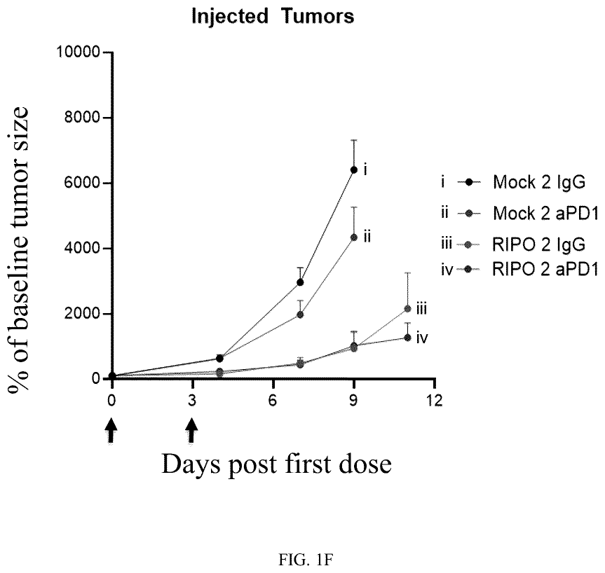

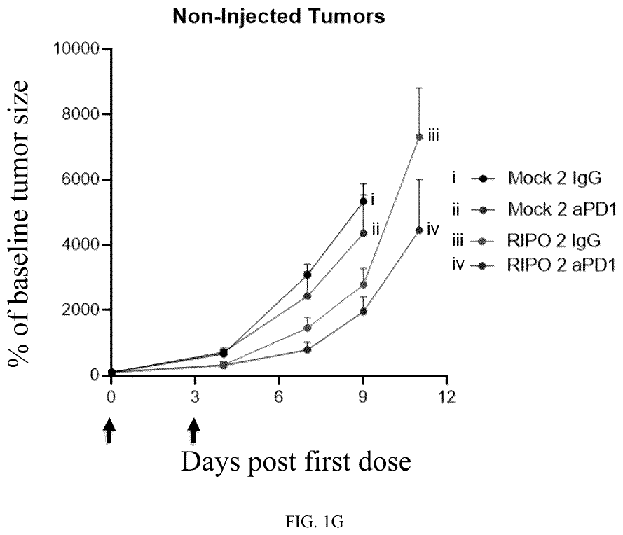

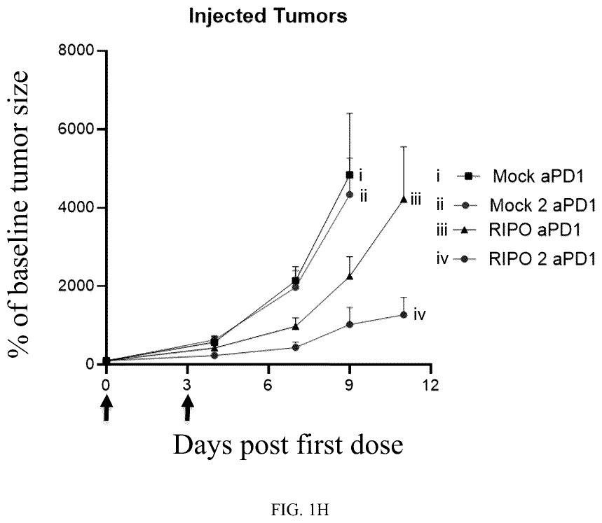

One day prior to cell implantation, mice (n=80) were shaved on bilateral flanks. The next day, mice were implanted with 2.5×10⁵ BV16-PVR cells in 50 μL PBS on each flank. One week following implantation, tumor sizes were measured using calipers, and treatment groups were randomized based upon the treated tumor volume. Beginning on day 7 following implantation, mice were administered intratumoral injections of either Mock (500 ul PBS) or PVSRIPO (1×10⁸ TCID₅₀/10 ul; Tox lot conc=2×10¹⁰ TCID₅₀/ml, lot #L0603006, diluted 1:1 in PBS; 250 ul PBS, 250 ul PVSRIPO) and intraperitoneal injections of either anti-PD-1 antibody (Bioxcell cat #BE0146; Lot #780120N1; Clone: RMP1-14; Stock concentration 10.57 mg/ml; Diluted to 2.5 mg/ml in PBS) or IgG (Bioxcell catalog #BE0089; Lot #686318F1; Clone: 2A3; Stock concentration: 7.35 mg/ml; Diluted to 2.5 mg/ml in PBS) control. See experimental plan summary in FIG. 1A.

TABLE 1
Exemplary Lerapolturev/PVSRIPO Sequences

| SEQ ID NO: | Sequence |
|---|---|
| 1- | ttaaaacagctctggggttgtacccaccccagaggcccacgtggcggctagtactccggtattgcggtacccttgtacgcctgttttatactcccttccc |
| Lerapolturev/ | gtaacttaggaattcaacttagaagtttttcacaaagaccaatagccggtaatcagccagattactgaaggtcaagcacttctgtttccccggtcaatgttg |
| PVSRIPO | atatgctccaacagggcaaaaacaactgcgatcgttaaccgcaaagcgcctacgcaaagcttagtagcatctttgaaatcgtttggctggtcgatccgc |
| | catttcccctggtagacctggcagatgaggctagaaataccccactggcgacagtgttctagcctgcgtggctgcctgcacaccctatgggtgtgaag |
| | ccaaacaatggacaaggtgtgaagagccccgtgtgctcgctttgagtcctccggcccctgaatgtggctaaccttaaccctgcagctagagcacgtaa |
| | cccaatgtgtatctagtcgtaatgagcaattgcgggatgggaccaactactttgggtgtccgtgtttcactttctcctttatatttgcttatggtgacaatatat |
| | acaatatatatattggcaccatgggagctcaggtttcatcacagaaagtgggcgcacatgaaaactcaaatagagcgtatggtggttctaccattaatta |
| | caccaccattaattattatagagattcagctagtaacgcggcttcgaaacaggacttctctcaagacccttccaagttcaccgagcccatcaaggatgtc |
| | ctgataaaaacatccccaatgctaaactcgccaaacatagaggcttgcgggtatagcgatagagtactgcaattaacactgggaaactccactataacc |
| | acacaggaggcggctaattcagtagtcgcttatgggcgttggcctgaatatctgagggacagcgaagccaatccagtggaccagccgacagaacca |
| | gacgtcgctgcatgcaggttttatacgctagacaccgtgtcttggacgaaagagtcgcgagggtggtggtggaagttgcctgatgcactgcgggacat |
| | gggactctttggccaaaatatgtactaccactacctaggtaggtccgggtacaccgtgcatgtacagtgtaacgcctccaaattccaccagggggcact |
| | aggggtattcgccgtaccagagatgtgtctggccggggatagcaacaccactaccatgcacaccagctatcaaaatgccaatcctggcgagaaagg |
| | aggcactttcacgggtacgttcactcctgacgacaaccagacatcacctgcccgtaggttctgcccggtggattacctctttggaaatggcacgttattg |
| | gggaatgcctttgtgttcccgcaccagataataaacctacggaccaacaactgtgctacactggtactcccttacgtgaactccctctcgatagatagtat |
| | ggtaaagcacaataattggggaattgcaatattaccattggccccattaaattttgctagtgagtcctccccagagattccaatcaccttgaccatagccc |
| | ctatgtgctgtgagttcaatggattaagaaacattaccctgccacgcttacagggcctgccggtcatgaacacccctggtagcaatcaatatcttactgc |
| | agacaacttccagtcaccgtgtgcgctgcctgaatttgatgtgaccccacctattgacatacccggtgaagttaagaacatgatggaattggcagaaat |
| | cgacaccatgattccctttgacttaagtgcaaaaaaaaagaacaccatggaaatgtatagggttcggttaagtgacaaaccacatacagacgatcccat |
| | actctgcctgtcactctctccagcttcagatcctaggttgtcacatactatgcttggagaaatcctaaattactacacacactgggcaggatccctgaagtt |
| | cacgtttctgttctgtggatccatgatggcaactggcaaactgttggtgtcatacgcgcctcctggagccgacccaccaaagaagcgtaaggaggcga |
| | tgttgggaacacatgtgatctgggacataggactgcagtcctcatgtactatggtagtgccatggattagcaacaccacgtatcggcaaaccatagatg |
| | atagtttcaccgaaggcggatacatcagcgtcttctaccaaaccagaatagtcgtccctctttcgacacccagagagatggacatccttggttttgtgtca |
| | gcgtgtaatgacttcagcgtgcgcttgatgcgagataccacacatatagagcaaaaagcgctagcacaggggttaggtcagatgcttgaaagcatgat |
| | tgacaacacagtccgtgaaacggtgggggcggcaacgtctagagacgctctcccaaacactgaagccagtggaccagcacactccaaggaaattc |
| | cggcactcaccgcagtggaaactggggccacaaatccactagtcccttctgatacagtgcaaaccagacatgttgtacaacataggtcaaggtcaga |
| | gtctagcatagagtctttcttcgcgcggggtgcatgcgtggccattataaccgtggataactcagcttccaccaagaataaggataagctatttacagtgt |
| | ggaagatcacttataaagatactgtccagttacggaggaaattggagttcttcacctattctagatttgatatggaatttacctttgtggttactgcaaatttca |
| | ctgagactaacaatgggcatgccttaaatcaagtgtaccaaattatgtacgtaccaccaggcgctccagtgcccgagaaatgggacgactacacatgg |
| | caaacctcatcaaatccatcaatcttttacacctacggaacagctccagcccggatctcggtaccgtatgttggtatttcgaacgcctattcacacttttac |
| | gacggtttttccaaagtaccactgaaggaccagtcggcagcactaggtgactccctctatggtgcagcatctctaaatgacttcggtattttggctgttag |
| | agtagtcaatgatcacaacccgaccaaggtcacctccaaaatcagagtgtatctaaaacccaaacacatcagagtctggtgcccgcgtccaccgagg |
| | gcagtggcgtactacggccctggagtggattacaaggatggtacgcttacacccctctccaccaaggatctgaccacatatggattcggacaccaaa |
| | acaaagcggtgtacactgcaggttacaaaatttgcaactaccatttggccactcaggaagatttgcaaaacgcagtgaacgtcatgtggaatagagac |
| | ctcttagtcacagaatcaagagcccagggcaccgattcaatcgcaaggtgcaattgcaacgcaggggtgtactactgcgagtctagaaggaaatact |
| | acccagtatccttcgttggcccaacgttccagtacatggaggctaataactattacccagctaggtaccagtcccatatgctcattggccatggattcgca |
| | tctccaggggattgtggtggcatactcagatgtcaccacggggtgatagggatcattactgctggtggagaagggttggttgcatttacagacattaga |
| | gacttgtatgcctacgaagaagaagccatggaacaaggcatcaccaattacatagagtcacttggggccgcatttggaagtggatttactcagcagatt |
| | ggagacaaaataacagagttgactaatatggtgaccagtaccatcactgaaaagctacttaagaacttgatcaagatcatatcctcactagttattataac |
| | taggaattatgaagacaccacaacagtgctcgctaccctggcccttcttgggtgtgatgcttcaccatggcagtggcttagaaagaaagcatgcgatgt |
| | tctggagataccttatgtcaccaagcaaggtgacagttggttgaagaagtttactgaagcatgcaacgcagctaagggactggagtgggtgtcaaaca |
| | aaatctcaaaattcattgattggctcaaggagaaaattatcccacaagctagagataagttggaatttgtaacaaaacttagacaactagaaatgctgga |
| | aaaccaaatctcaactatacaccaatcatgccctagtcaggaacaccaggaaattctattcaataatgtcagatggttatccatccagtctaagaggtttg |
| | cccctctttacgcagtggaagccaaaagaatacagaaactagagcataccattaacaactacatacagttcaagagcaaacaccgtattgaaccagtat |
| | gtttgctagtacatggcagccccggaacaggtaaatctgtagcaaccaacctgattgctagagccatagctgaaagagaaaacacgtccacgtactcg |
| | ctacccccggatccatcacacttcgacggatacaaacaacagggagtggtgattatggacgacctgaatcaaaacccagatggtgcggacatgaag |
| | ctgttctgtcagatggtatcaacagtggagtttataccacccatggcatccctggaggagaaaggaatcctgtttacttcaaattacgttctagcatccac |
| | gaactcaagcagaatttccccccccactgtggcacacagtgatgcattagccaggcgctttgcgttcgacatggacattcaggtcatgaatgagtattct |
| | agagatgggaaattgaacatggccatggctactgaaatgtgtaagaactgtcaccaaccagcaaactttaagagatgctgtcctttagtgtgtggtaag |
| | gcaattcaattaatggataaatcttccagagttagatacagtattgaccagatcactacaatgattatcaatgagagaaacagaagatccaacattggcaa |
| | ttgtatggaggctttgttccaaggaccactccagtataaagacttgaagattgacatcaagacgagtccccctcctgaatgtatcaatgacttgctccaag |
| | cagttgactcccaggaggtgagagattactgtgagaagaagggttggatagtcaacatcaccagccaggttcaaacagaaaggaacatcaacaggg |
| | caatgacaattctacaagcggtgacaaccttcgccgcagtggctggagttgtctatgtcatgtataaactgtttgctggacaccagggagcatacactg |
| | gtttaccaaacaaaaaacccaacgtgcccaccattaggacagcaaaggtacaagggccagggttcgattacgcagtggctatggctaaaagaaacat |
| | tgttacagcaactactagcaagggagagttcactatgttaggagtccacgacaacgtggctattttaccaacccacgcttcacctggtgaaagcattgtg |
| | atcgatggcaaagaagtggagatcttggatgccaaagcgctcgaagatcaagcaggaaccaatcttgaaatcactataatcactctaaagagaaatga |
| | aaagttcagagacattagaccacatatacctactcaaatcactgagacaaatgatggagtcttgatcgtgaacactagcaagtaccccaatatgtatgttc |
| | ctgtcggtgctgtgactgaacagggatatctaaatctcggtgggcgccaaactgctcgtactctaatgtacaactttccaaccagagcaggacagtgtg |
| | gtggagtcatcacatgtactgggaaagtcatcgggatgcatgttggtgggaacggttcacacgggtttgcagcggccctgaagcgatcatacttcact |
| | cagagtcaaggtgaaatccagtggatgagaccttcgaaggaagtgggatatccaatcataaatgccccgtccaaaaccaagcttgaacccagtgcttt |
| | ccactatgtgtttgaaggggtgaaggaaccagcagtcctcactaaaaacgatcccaggcttaagacaaactttgaggaggcaattttctccaagtacgt |
| | gggtaacaaaattactgaagtggatgagcacatgaaagaggcagtagaccactatgctggccagctcatgtcactagacatcaacacagaacaaatg |
| | tgcttggaggatgccatgtatggcactgatggtctagaagcacttgatttgtccaccagtgctggctacccttatgtagcaatgggaaagaagaagaga |
| | gatatcttgaacaaacaaaccagagacactaaggaaatgcaaaaactgctcgacacatatggaatcaacctcccactggtgacttatgtaaaggatga |
| | acttagatccaaaacaaaggttgagcaggggaaatccagattaattgaagcttctagtttgaatgactcagtggcaatgagaatggcttttgggaaccta |
| | tatgctgcttttcacaaaaacccaggagtgataacaggttcagcagtagggtgcgatccagatttgttttggagcaaaattccggtattgatggaagaga |
| | agctgtttgcctttgactacacagggtatgatgcatctctcagccctgcttggttcgaggcactaaagatggtgcttgagaaaatcggattcggagacag |
| | agttgactacatcgactacctaaaccactcacaccacctgtacaagaataaaacatactgtgtcaagggcggtatgccatctggttgctcaggcacttca |
| | atttttaactcaatgattaacaacttgattatcaggacactcttactgaaaacctacaagggcatagatttagaccacctaaaaatgattgcctatggtgatg |
| | atgtaattgcttcctacccccatgaagttgacgctagtctcctagcccaatcaggaaaagactatggactaactatgactccagctgacaaatcagctata |
| | tttgaaacagtcacatgggagaatgtaacattcttgaagagattcttcagggcagacgagaaatacccatttcttattcatccagtaatgccaatgaagga |
| | aattcatgaatcaattagatggacaaaagatcctaggaacactcaggatcacgttcgctctctgtgcctattagcttggcacaatggcgaagaagaatat |
| | aacaaattcctagctaaaatcaggagtgtgccaattggaagagctttattgctcccagagtactcaacattgtaccgccgttggcttgactcattttagtaa |
| | ccctacctcagtcgaattggattgggtcatactgctgtaggggtaaatttttctttaattcggagaaaaaaaaaaaaaccc |
| 2-5′ | ttaaaacagctctggggttgtacccaccccagaggcccacgtggcggctagtactccggtattgcggtacccttgtacgcctgttttatactcccttccc |
| cloverleaf | gtaacttag |
| 3- | gaattcaacttagaagtttttcacaaagaccaatagccggtaatcagccagattactgaaggtcaagcacttctgtttccccggtcaatgttgatatgctcc |
| human | aacagggcaaaaacaactgcgatcgttaaccgcaaagcgcctacgcaaagcttagtagcatctttgaaatcgtttggctggtcgatccgccatttcccc |
| rhinovirus 2 | tggtagacctggcagatgaggctagaaataccccactggcgacagtgttctagcctgcgtggctgcctgcacaccctatgggtgtgaagccaaacaa |
| (HRV2) | tggacaaggtgtgaagagccccgtgtgctcgctttgagtcctccggcccctgaatgtggctaaccttaaccctgcagctagagcacgtaacccaatgt |
| internal | gtatctagtcgtaatgagcaattgcgggatgggaccaactactttgggtgtccgtgtttcactttctcctttatatttgcttatggtgacaatatatacaatatat |
| ribosomal | atattggcacc |
| entry site | |
| (IRES) | |

Four days later, tumor sizes were measured, then mice were administered intratumoral injections of either Mock or PVSRIPO with repeat anti-PD-1 antibody or IgG dosing. After three days, tumor sizes were measured, then a final repeat anti-PD-1 antibody or IgG dosing was performed. Four consecutive tumor size measurements were obtained at day 9, day 11, day 14, day 17 post-initial administrations. Following this, mice were euthanized upon ulceration or total tumor size (both flanks) exceeding 1000 mm³. Results at Day 9 post-first Mock/PVSRIPO dose administration show that PVSRIPO in combination with anti-PD-1 antibody treatment provides reduced tumor progression in injected lesions than either monotherapy alone (FIG. 1B). Importantly, two doses of PVSRIPO in combination with anti-PD-1 antibody provides better tumor growth suppression in injected tumors when compared to all other treatment groups. Not only were excellent results observed for tumors multiply injected with PVSRIPO, but other non-treated metastatic lesions distant from the injected tumor sites also experienced reduced tumor growth at Day 9 following two doses of PVSRIPO in combination with anti-PD-1 antibody (FIG. 1C), referred to as an “abscopal effect”. The responses observed in untreated (non-infused/non-injected) lesions of B16/OVA melanoma model mice were unexpected since the PVSRIPO was administered to a distant lesion, i.e., a distinct tumor, and suggest the combination of PVSRIPO and ICI therapy provokes a systemic anti-tumor immune response. Two doses of PVSRIPO demonstrated better efficacy in injected lesions (FIG. 1D) when compared to a single dose. No difference was observed between one dose and two doses of PVSRIPO monotherapy in non-injected tumors (FIG. 1E), which showed significant reduction compared to mock administration. However, when PVSRIPO is administered in combination with anti-PD-1 antibody treatment tumor potency is increased (FIG. 1F-G). The addition of anti-PD-1 antibody to PVSRIPO treatment enhances efficacy in non-injected lesions (FIG. 1G). When considering all treatment groups that include anti-PD-1 antibody administration, two doses of PVSRIPO provides the best efficacy in both injected (FIG. 1H) and non-injected (FIG. 1I) lesions.

These data suggest administration of multiple doses of PVSRIPO are feasible and unexpectedly provide efficacious anti-tumor responses that are further enhanced when combined with anti-PD-1 treatment regimens.

### Example 2. Trial Involving an Increased Lerapolturev Dosage and Dosing Frequency Treatment Regimen

A multi-center, open-label, randomized, Phase 2 study will investigate the efficacy and safety of lerapolturev/PVSRIPO alone (Arm 1) or in combination with an anti-PD-1 inhibitor (Arm 2). Following a 6-participant safety run-in period, up to approximately 50 participants with unresectable cutaneous melanoma who previously failed an PD-1/PD-L1 inhibitor-based therapy will be randomized 1:1 to receive either lerapolturev or lerapolturev plus an PD-1 inhibitor. Based on updated eligibility criteria, participants will be stratified based on type of PD-1/PD-L1 inhibitor resistance (i.e., primary versus secondary) (Kluger et al. J Immunother Cancer. 8(1):e000398(2020)) and baseline lactate dehydrogenase (LDH) levels (normal versus>ULN).

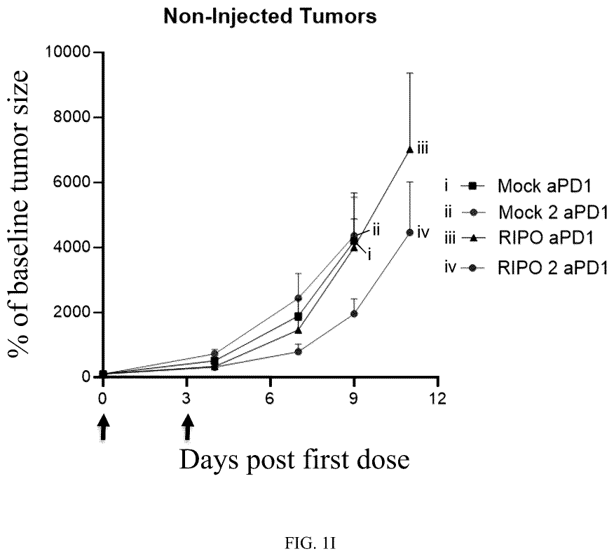

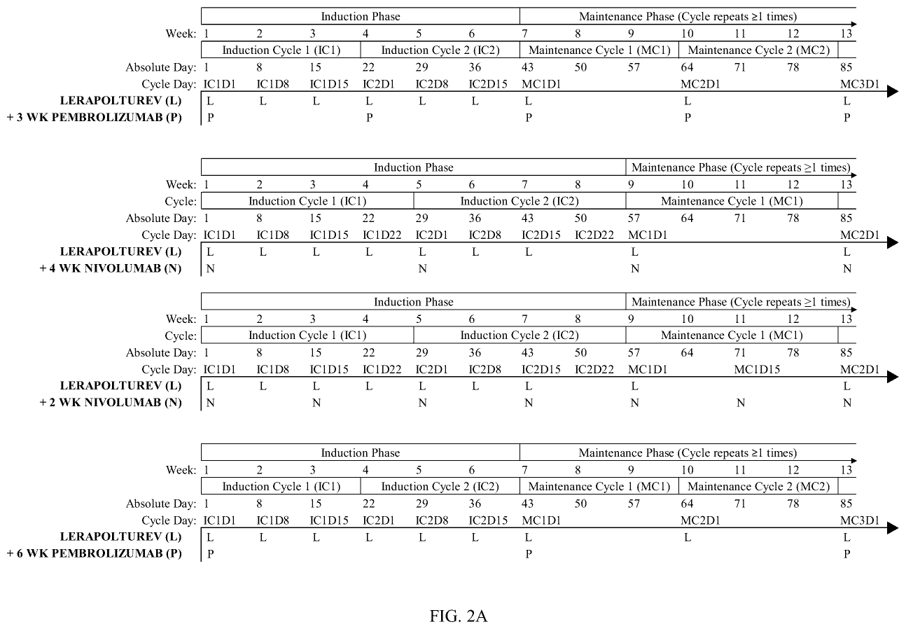

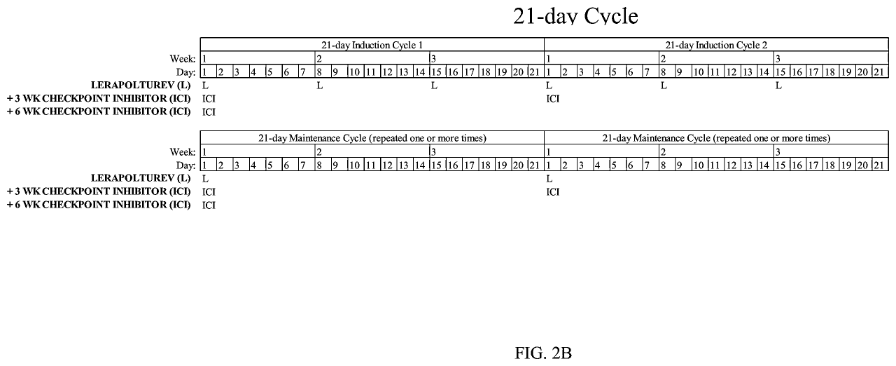

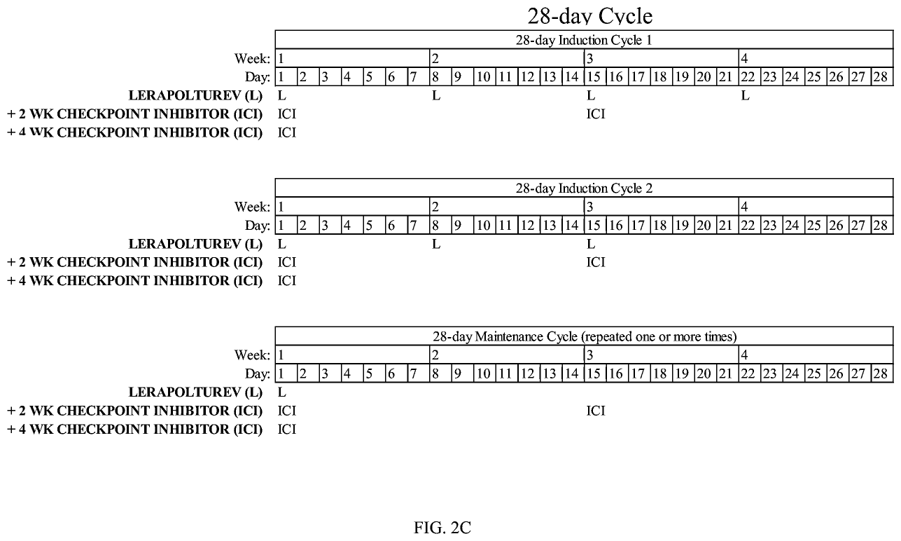

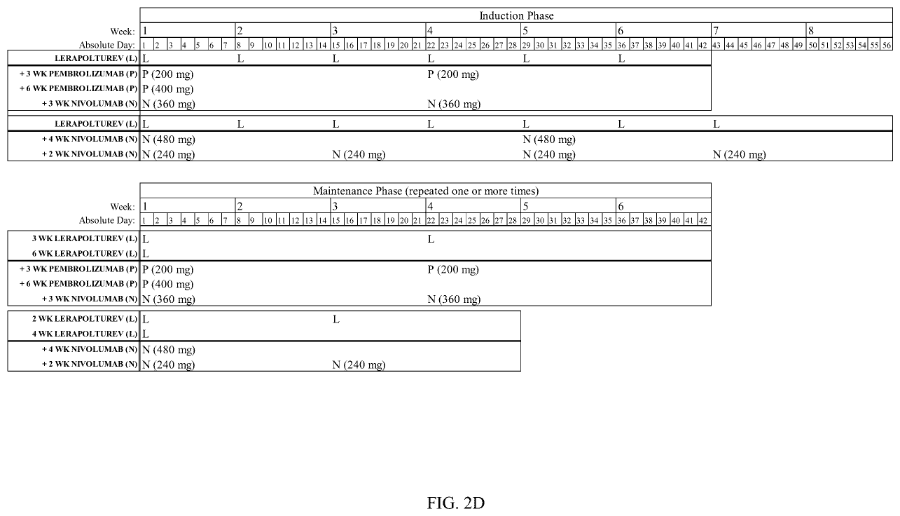

The total lerapolturev dose to be administered will be a maximum of 1.6×10⁹ TCID₅₀/visit, with up to 6 lesions injected/visit. Previously, lerapolturev doses were administered at a maximum of 6×10⁸ TCID₅₀/visit. Lerapolturev injection volumes will be stratified depending on the size of the lesion to be injected (Table 2). The dose schedule of administration will include 7 weekly lerapolturev injections followed by either every three-week dosing (lerapolturev monotherapy or in combination with pembrolizumab) or every 4-week dosing (combination with nivolumab) or every two-week dosing (combination with nivolumab) or every six-week dosing (combination with pembrolizumab) (FIG. 2A). The dose schedules of administration are separated into two different phases including an Induction Phase and a Maintenance Phase. The Induction Phase includes either 21-day Induction Cycles (FIG. 2B) or 28-day Induction Cycles (FIG. 2C) wherein lerapolturev is administered weekly with an ICI cycling at 2 weeks, 3 weeks, or 4 weeks, or 6 weeks. Different possible schedules of lerapolturev with different cycling/dosages of pembrolizumab or nivolumab are illustrated in FIG. 2D. The Maintenance Phase comprises one or more cycles of a lerapolturev administration with a cycling ICI (e.g., pembrolizumab, nivolumab) per the package insert of the label of the ICI(s).

TABLE 2
Recommended Lerapolturev Injection Volumes

| | Maximum | Lerapolturev |
|---|---|---|
| Lesion Size | Injection Volume | Concentration |
| (longest dimension [cm]) | Per Lesion (ml) | (TCID₅₀/ml) |
| ≥5 | 3.0 | 5.33 × 10⁸ |
| ≥4 and <5 | 2.0 | |
| ≥3 and <4 | 1.5 | |
| ≥2 and <3 | 1.0 | |
| 0.5 to <2.0 | 0.5 | |

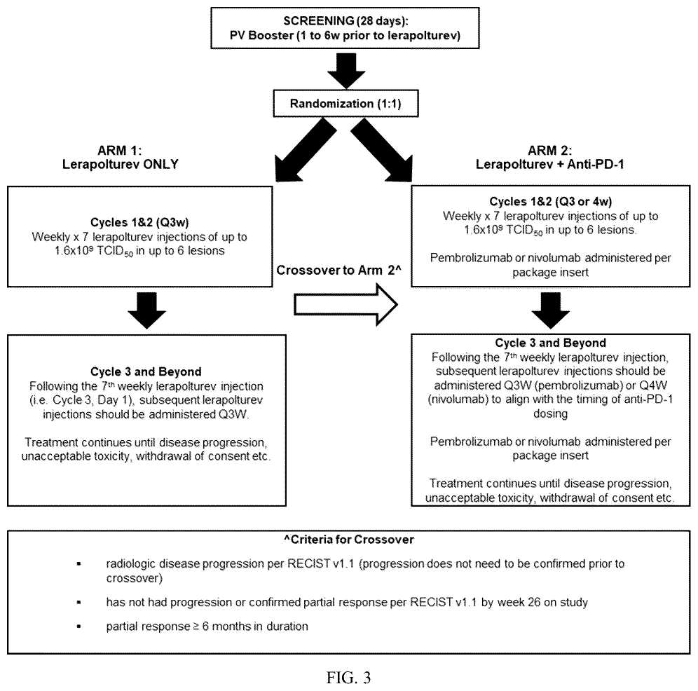

Participants enrolled in a safety run-in, or those randomized to Arm 1 (lerapolturev only), are allowed to crossover to Arm 2 following disease progression (per RECIST 1.1), once a PR lasts ≥6 months, or after 26 weeks on study without RECIST 1.1-defined progression (i.e., SD or unconfirmed PR or CR) (FIG. 3). In Arm 2, PD-1 inhibitor will be administered per the manufacturer's prescribing information concurrently with lerapolturev beginning Day 1. After 12 participants have been randomized and on study approximately 21 days (i.e., once n=6 participants per treatment arm receiving lerapolturev weekly have completed the DLT evaluation period), an additional safety review will be performed by a data safety monitoring committee to confirm the safety of lerapolturev with or without PD-1 inhibitor and/or to make recommendations related to lerapolturev dose and/or schedule adjustments.

Participants will be evaluated for improved patient outcomes for example, key primary and secondary anti-tumor response endpoints based on RECIST 1.1. Additional primary endpoints include characterization of the immunologic response (e.g., changes in CD8+ TIL levels and PD-L1 expression) to lerapolturev with and without PD-1 inhibitor and safety. Because lerapolturev is an immunotherapeutic, participant management with respect to treatment decisions (e.g., confirmation of PD for study discontinuation) will occur based on iRECIST criteria. However, participants in the safety run-in or Arm 1 are allowed to crossover to Arm 2 upon RECIST-defined PD, in which the any one of the following criteria are met: (i) radiologic disease progression per RECIST 1.1 (progression does not need to be confirmed prior to crossover), (ii) have not had progression or confirmed PR per RECIST 1.1 by week 26 on study, (iii) PR≥6 months in duration. All participants crossing over to combination therapy will have a complete assessment of tumor burden (i.e., scans and caliper/ruler measurement of skin lesions with photographic documentation)≤28 days prior to starting the combination. The dose of lerapolturev administered in combination will be the same as monotherapy. The schedule of lerapolturev injections at the time of initiation of combination therapy will depend on the timing of crossover (FIG. 3). ORR, DOR, and DRR based on iRECIST are exploratory endpoints.

### Example 3. Lesion Changes in Patients Receiving Improved Lerapolturev Dosage Regimen

The new protocol amendment was implemented in January 2022 with patients receiving 2.5-fold more lerapolturev per visit (1.6×10⁹ TCID₅₀) relative to the previous amendment. The protocol (LUMINOS-102) was originally designed to test lerapolturev injections in up to 6 lesions (or max dose of 6×10⁸ TCID₅₀) given every 3 to 4 weeks (Q3/4W schedule) with or without anti-PD-1 antibody therapy. As of a 10 Dec. 2021 data cutoff, all AEs related to lerapolturev or anti-PD-1 therapy remained Grade 1 or 2. No dose-limiting toxicities, treatment-related SAEs, or signs and symptoms of cytokine release syndrome (CRS) were reported in the 18 participants treated despite a 6-fold increase in lerapolturev dose relative to the original dose evaluated in the Phase 1 study. While the data from LUMINOS-102 had shown lerapolturev to be safe and tolerable, the level of clinical activity observed in the Phase 1 study was not yet duplicated (as of December 2021). Therefore, to improve the probability of patient benefit and the resultant benefit/risk profile, both the eligibility criteria and dose/dosing regimen were updated in the improved protocol implemented during January 2022.

TABLE 3
Protocol amendment implementation (as of January 2023)

| | Initially | | |
|---|---|---|---|
| | consented to | | New patients |
| | previous | Reconsented to | consented to new |
| | amendment | new amendment | amendment |
| Patients | 20 | 6 of 20 | 4 |
| Treated (n) | | | |
| Max Lera dose | 6 × 10⁸ TCID₅₀ | 1.6 × 10⁹ TCID₅₀ | 1.6 × 10⁹ TCID₅₀ |
| Lera Schedule | Q3 or 4 weeks | Q3 or 4 weeks | 7× weekly followed |
| | | | by Q3 or 4W |
| | | | maintenance |

### Abscopal Response in 25-Year-Old Male With BRAF Mutant (BRAF+) Melanoma

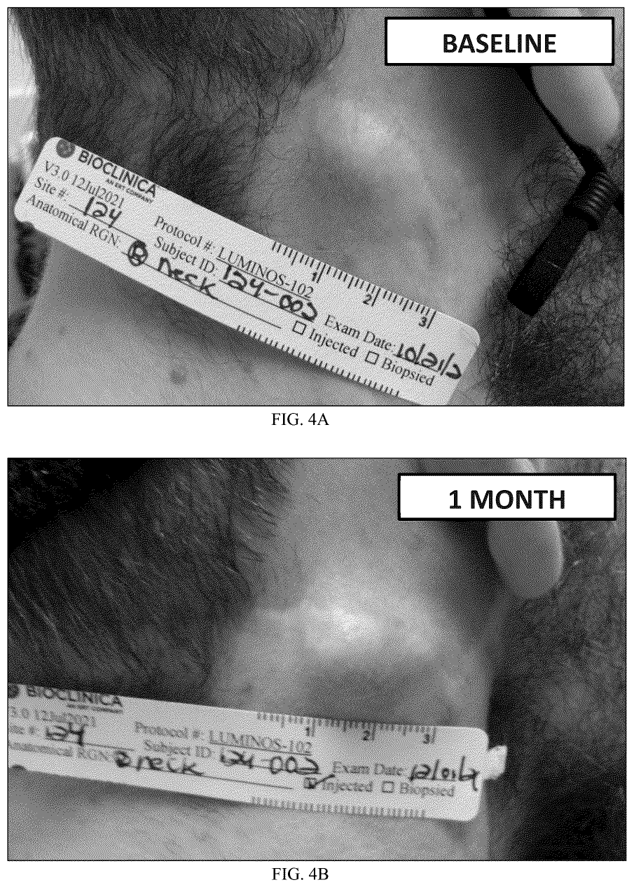

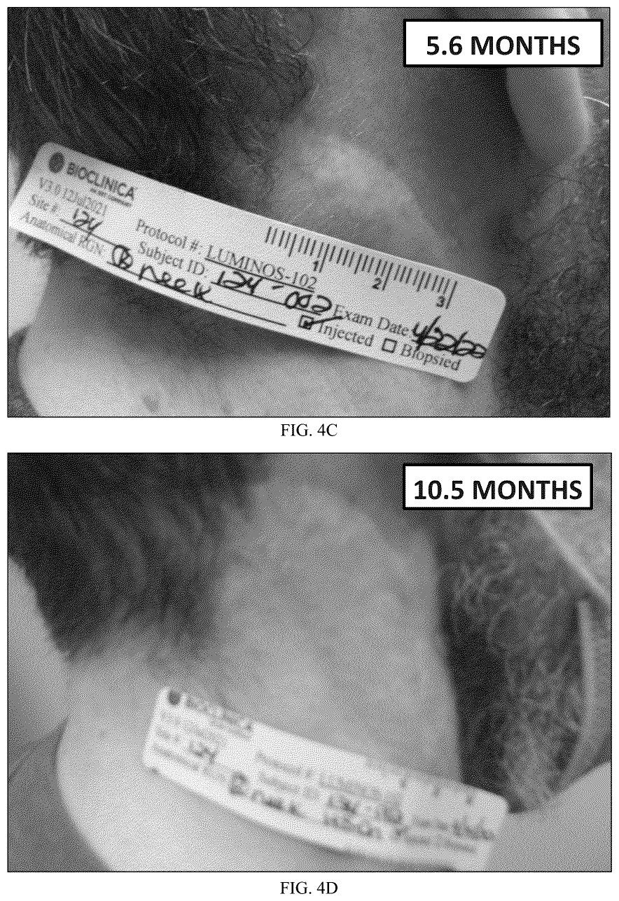

Between November 2019 and April 2020, a patient with initial Stage IIIB melanoma received wide local excision with right anterolateral neck dissection followed by adjuvant nivolumab. Between June 2020 and June 2021, the patient experienced recurrence in the right side of the neck and was treated with deep cervical lymph node excision and adjuvant BRAK/MEK inhibitors. In September 2021, the patient experienced recurrence with Stage IV MIB melanoma lesions in the right neck and right lung. Under the previous protocol amendment, the patient began lerapolturev monotherapy as a part of Arm 1 on cycle 1 day 1 (C1D1) in November 2021. The injected lesion initially increased in size (FIG. 4A-B). After spending 3.5 months on lerapolturev monotherapy at the original dose level, this patient was crossed over to combination therapy with an increase dose of lerapolturev in February 2022. The patient began to see improvements in the injected neck lesion at 5.6 months on study (FIG. 4C) with the right neck lesion not being palpable. After being on the study for 10.5 months, the right neck lesion continued to improve and was still not palpable (FIG. 4D). An abscopal response was also observed in the patient, with the disappearance of a lesion in his right lung (FIG. 4E). The patient has received 5 monotherapy injections and 4 injections in combination with pembrolizumab.

### Complete Response in 66-Year-Old Male Diagnosed With BRAF Mutant (BRAF+) Melanoma

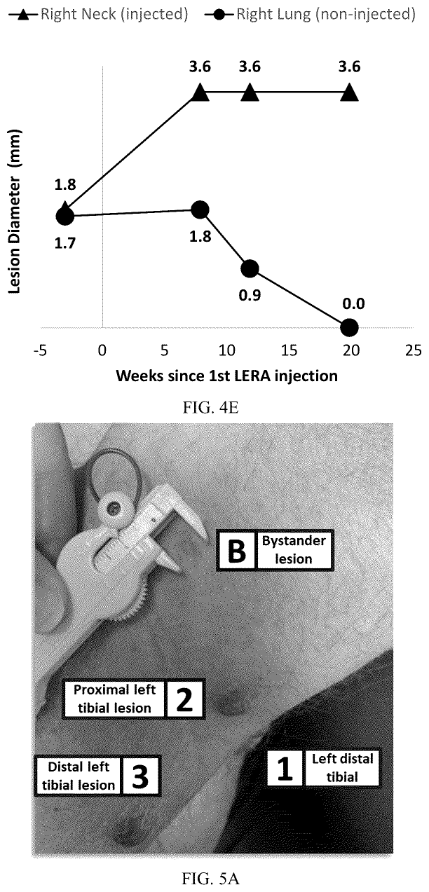

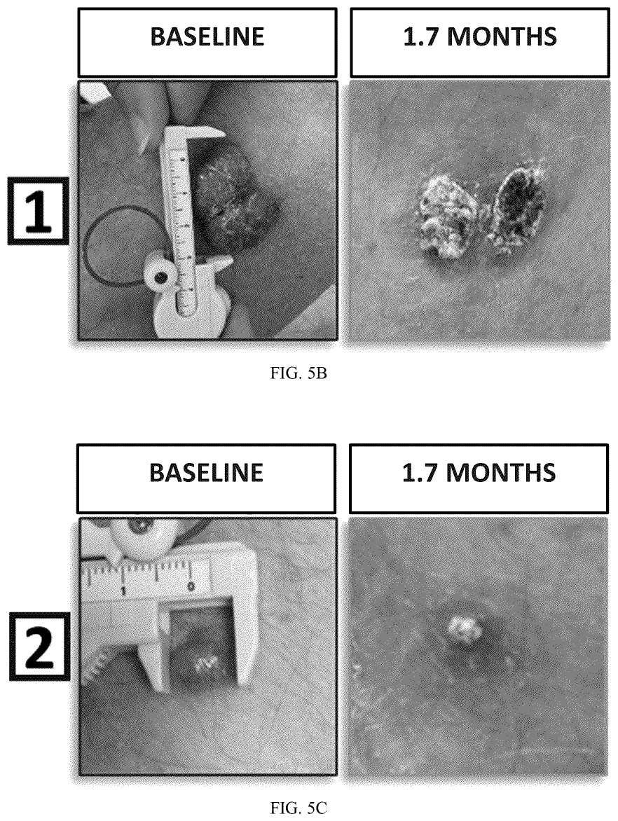

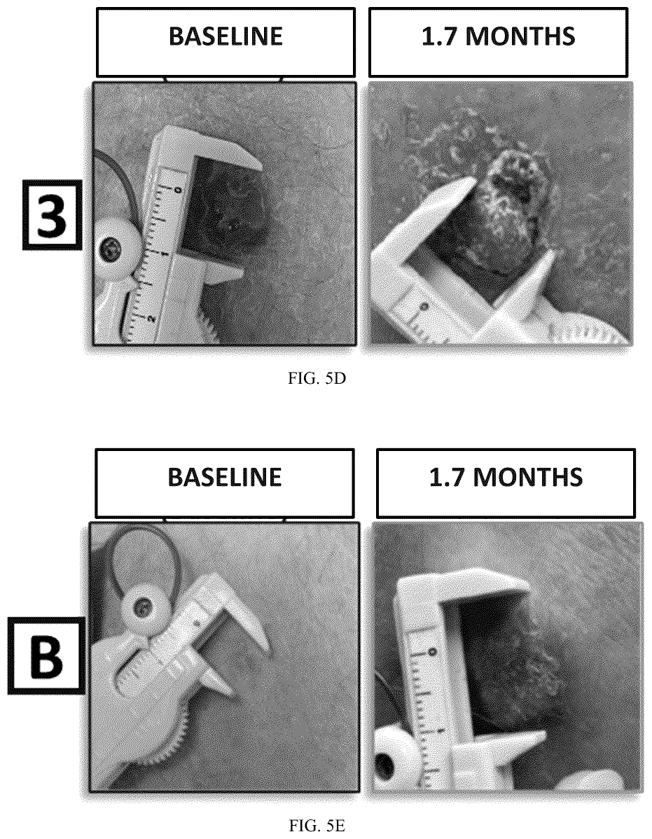

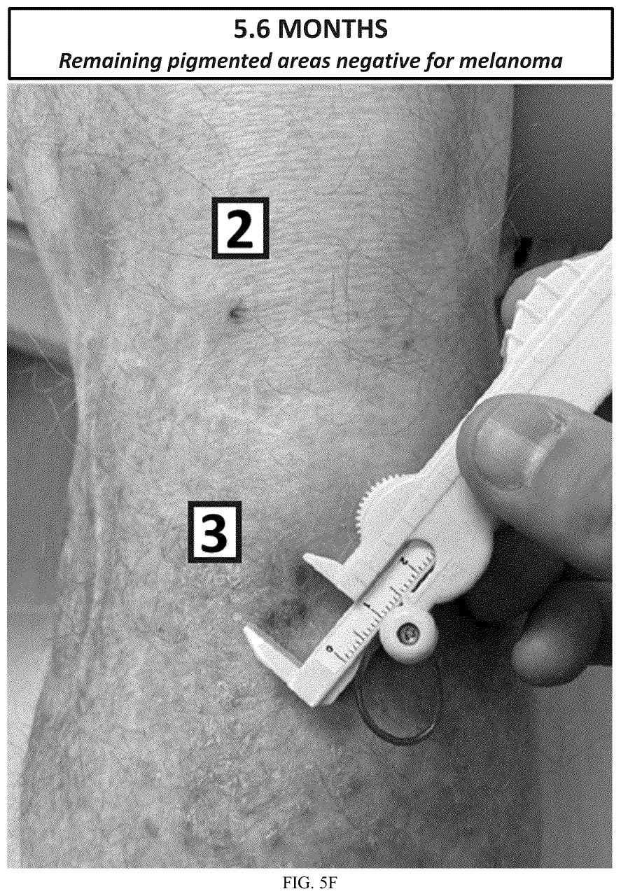

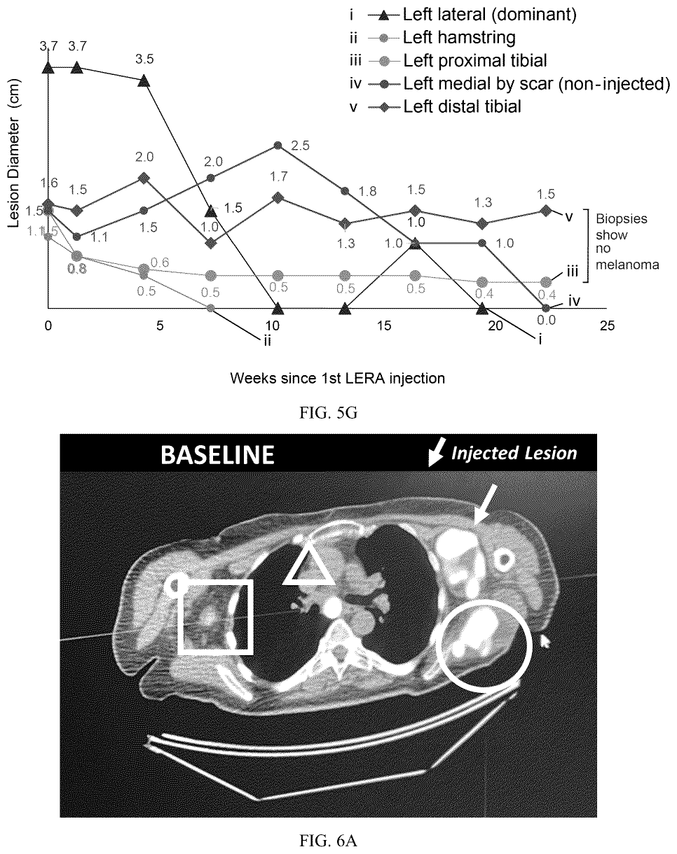

In 2011, a patient with BRAF mutant (BRAF+) melanoma had his left second toe amputated. Between January 2020 and September 2020, the patient experienced in transit disease recurrence with a left knee lesion treated with wide local excision and adjuvant pembrolizumab. In September 2020, the patient experienced recurrence and a left leg lesion was resected. Between May 2021 and August 2021, the patient once again experienced recurrence with satellite lesions that were treated with resection and ipilimumab and nivolumab. In November 2021, the patient began cycle 1 day 1 (C1D1) lerapolturev combination therapy in Arm 2 of the previous protocol amendment. Between November 2021 and April 2022, the patient had received a total of 9 lerapolturev injections with all lesions having received at least one lerapolturev injection except the hamstring lesion (FIG. 5A-E). The patient switched over to the increased dose of lerapolturev monotherapy resulting in a complete response, as 3 of 5 in-transit lesions completely disappeared, and the other two lesions testing negative for melanoma as described below. During the patient's 3 months on lerapolturev monotherapy at the original dose, this patient began to see a response in injected and non-injected lesions. Biopsies of the remaining 2 show no evidence of melanoma (FIG. 5F-G).

### Partial Response in 56-Year-Old Female With NRAS Mutant (NRAS+) Melanoma

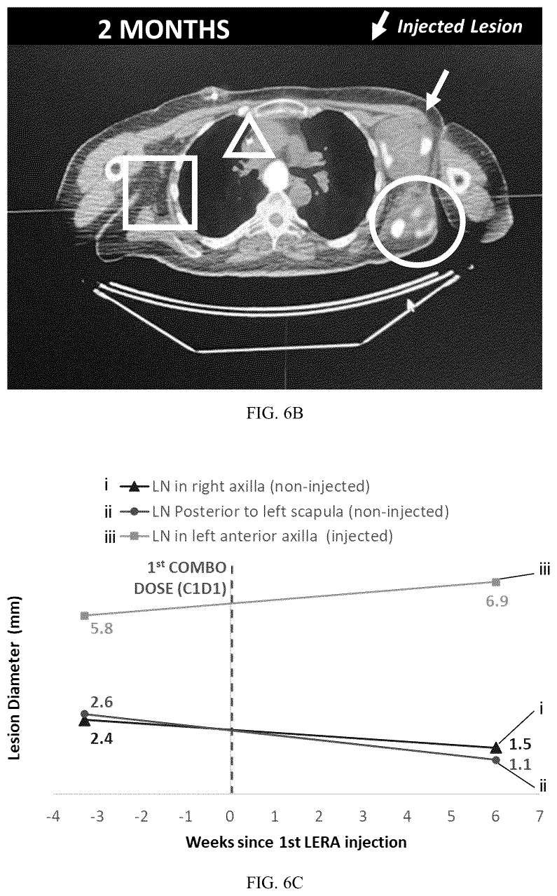

Between January 2021 and January 2022, a patient with NRAS mutant (NRAS+) melanoma had masses excised in the left mid-back and left axilla, followed by adjuvant nivolumab therapy. The patient then received two cycles of ipilimumab concurrent with nivolumab, which was discontinued due to arthralgia (FIG. 6A). In March of 2022, the patient began lerapolturev induction treatment (7× weekly injections) in combination with anti-PD-1 therapy. In April 2022, the patient had completed the 7 weeks of induction therapy in which a maximum allowable dose of 1.6×10⁹ TCID₅₀ (3.0 ml) was administered throughout induction. The patient has a total of 4 lesions with no sites of visceral disease (FIG. 6B). The injected lesion became edematous with a decrease in metabolically active cells in the center, suggesting necrosis. A total of 2 of 3 target lesions (both non-injected) regressed ˜50% at first scan (FIG. 6C), suggesting an abscopal response. The subcarinal lymph node (LN) lesion, however, appeared larger.

### Validation of Lerapolturev Viral Replication in the Tumor Microenvironment

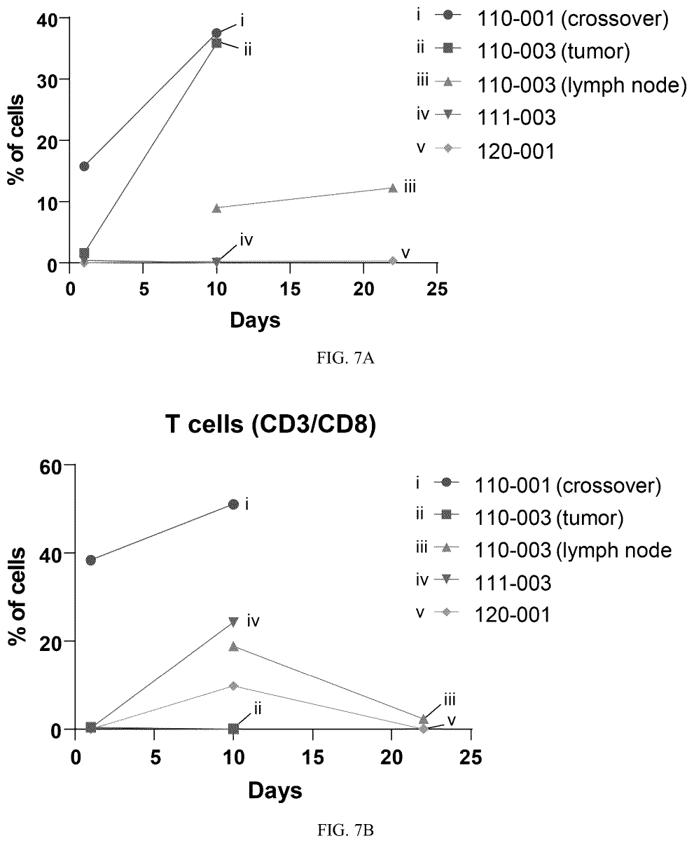

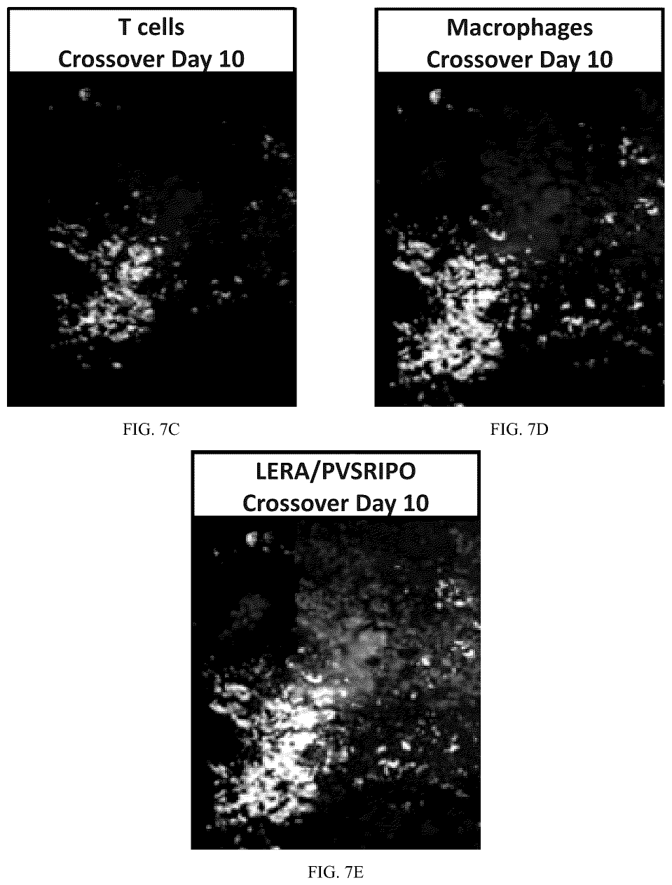

Viral replication analysis was tested in samples isolated from four patients having unresectable anti-PD-1 refractory melanoma (110-001, 110-003, 111-003, 120-001) over 22 days (Table 4, FIGS. 7A-7E). Two patients began on lerapolturev monotherapy (110-001, 120-001), whereas the other two patients began on lerapolturev and PD-1 inhibitor combination therapy (110-003, 111-003). Patient 110-003 received nivolumab while Patient 111-003 received pembrolizumab in addition to lerapolturev.

TABLE 4
Sample collection times of FIG. 7A-7E

| | | Day 1 | | Visit Date | Study |
|---|---|---|---|---|---|
| Subject | Sample | (mm-dd-yyyy) | Timepoints | (mm-dd-yyyy) | Day |
| 110-001 | Tumor | Mar. 30, 2021 | Crossover | Sep. 28, 2021 | 183 |
| | | | Day 0 | | |
| | | | Crossover | Oct. 8, 2021 | 193 |
| | | | Day 10 | | |
| 110-003 | Tumor | Sep. 15, 2021 | Baseline Day 0 | Sep. 15, 2021 | 1 |
| | | | Cycle 2 Day 10 | Sep. 24, 2021 | 10 |
| 110-003 | Lymph | Sep. 15, 2021 | Cycle 2 Day 10 | Sep. 24, 2021 | 10 |
| | node | | Cycle 2 Day 22 | Oct. 4, 2021 | 20 |
| 111-003 | Tumor | Aug. 12, 2021 | Baseline Day 0 | Jul. 22, 2021 | −20 |
| | | | Cycle 2 Day 10 | Aug. 23, 2021 | 12 |
| | | | Cycle 2 Day 22 | Sep. 2, 2021 | 22 |
| 120-001 | Tumor | Aug. 12, 2021 | Baseline Day 0 | Jul. 22, 2021 | −20 |
| | | | Cycle 2 Day 10 | Aug. 23, 2021 | 12 |
| | | | Cycle 2 Day 22 | Sep. 2, 2021 | 22 |

Samples were formalin fixed and paraffin embedded (FFPE) and sectioned at 5 μm for downstream immunolabel-based image analysis. Briefly, the samples were de-waxed by incubating in XS-3 for 30 minutes for two cycles, then rehydrated in a stepwise gradient with 100%, 95%, 70%, and 50% reagent alcohol for 10 minutes each. The slides were then incubated in Milli-Q water for five minutes for three cycles to complete hydration. The samples were then heated in 1× citrate buffer in boiling water for 20 minutes to expose antigen binding sites, followed by two washes with TBS-T (TBS+0.2% Triton X) and three washes with TBS. Cell counts for each population of total cells and immune cell subpopulations were conducted by labeling with tumor epithelial stromal marker PanCK. In addition to this, the plus (+) and minus (−) RNA strands of lerapolturev (PVSRIPO) used in the therapy was probed, amplified, and labeled in situ on the FFPE section to detect viral infection and replication respectively.

The immunolabeling workflow began with blocking the tissue in 6% donkey serum in TBST (TBS+0.2% Triton X) with 0.3 M glycine for 1 hour, followed by overnight incubation of the primary antibody and 1-hour of secondary antibody incubation. The concentrations used for the primary antibodies was 1:100 except for CD3 which was 1:50. Whenever directly conjugated primary antibodies were used in the panels, it was done after secondary labeling and double blocking with 6% normal rabbit serum and normal mouse serum for 30 minutes. Details are included below (Table 5).

TABLE 5
Panels for immunolabeling FFPE samples

| Immuno- | Primary | Vendor and | | |
|---|---|---|---|---|
| Target | Ab | SKU | Secondary Ab | Vendor and SKU |
| Macrophages | CD68 | NOVUS, | Directly conjugated to 488 | N/A |
| | | NB110- | | |
| | | 89474AF594 | | |
| Cytotoxic T | CD3 | Abcam, | Donkey anti rabbit 555 | ThermoFisher, |
| cells | | ab135372 | | A31572 |
| | CD8a | Abcam, | Donkey anti rabbit 555 | ThermoFisher, |
| | | ab209775 | | A31572 |
| Tumor | PanCK | ORIGENE, | Donkey Anti guinea pig 555 | SIGMA- |
| Epithelial Cells | | BP5069 | | SAB4600297 |
| Lerapolturev | Probe B2 | Molecular | Poliovirus_PV_S1_minus_B2 488 | Molecular |
| Viral | | Instruments | | Instruments |
| Replication | | | | |

The concentrations used for all secondaries was 2.5 μg/ml. Hoechst (1:25,000) was used for the nuclear stain during the secondary antibody incubation and after staining, the slides were cover slipped with an aqueous based mounting medium Fluorogel (Electron Microscopy Sciences: 50-247-04). After imaging of the first panel, the antibodies were removed using Visikol's proprietary EasyPlex technology that removes the primary and secondary antibodies from the tissue for further immunolabeling and multiplexing of the tissues. After antibody stripping, the tissue sections were then reblocked in 6% donkey serum in TBS-T (TBS+0.2% Triton X) with 0.3 M of glycine for 1 hour, where the samples were multiplexed using the previously mentioned protocol.

For HCR-RNA FISH a modified protocol from Molecular Instruments was used to label the samples. The HCR RNA FISH Probe B2 (Poliovirus_PV_S1_minus_B2_488), which detects the minus strand of PVSRIPO, was used as an indicator of replication. During pretreatment of the samples and prior to probe hybridization the samples were permeabilized stepwise in 50%, 70% and 100% methanol in 1×PBS for 10 mins each and rehydrated stepwise in 100%, 70% and 50% methanol in 1×PBS. Following this, the samples were further permeabilized in 1×PBS-T, then in 1×SSCT for 10 mins at RT. The probes were diluted at the suggested 16 nM concentration in probe hybridization buffer and the hybridization of the probes on the samples was performed overnight at 37° C. with gentle shaking in a humidified chamber. The next day, prior to amplification, the excess probes were washed and removed in a stepwise manner by washing the slides in 100% probe wash buffer for 15 min, then they were washed in 75% probe wash buffer/25% 5×SSCT for 15 mins, then in 50% probe wash buffer/50% 5×SSCT for 15 mins then again 25% probe wash buffer/75% 5×SSCT for 15 mins. The final wash was done in 100% 5×SSCT for 15 mins.

Prior to probe amplification, pre-amplification was performed by adding amplification buffer on top of the tissue samples in a humidified chamber for 30 mins at RT. The Alexa Fluor conjugated amplification hairpins, hairpin h1 and hairpin h2 were snap cooled and diluted to a 3 uM stock, then heated at 95° C. for 90 seconds. The slides continued to be incubated at room temperature in a dark drawer for 30 mins. After the RT incubation, the hairpin solution was prepared by adding the snap-cooled h1 and h2 hairpins to amplification buffer at room temperature. After removing the pre amplification buffer from the samples, the hairpin solution was added on top of the tissue sample, where the amount of hairpin solution used depended on the size of the tissue. The amplification was performed by incubating the samples overnight in a dark humidified chamber at room temperature. The next day, the excess hairpins were removed by washing the slides five times in 5×SSCT at room temperature for 10 mins each. Hoechst (1:25,000) was used for the nuclear staining the slides and then were cover-slipped with aqueous based mounting medium Fluorogel in 1×TBS (Electron Microscopy Sciences: 50-247-04).

The slides were imaged using an Aperio Leica Versa 8 (Leica Biosystems) Slide Scanner at 40× magnification (0.1625 um/pixel, NA=0.85). All images were uploaded to BitSlide for viewing and downloading.

Patients 110-001 and 110-003 (tumor sample) exhibited an increased percent of cells replicating the lerapolturev virus in the lesion sample (FIG. 7A) from day 1 to day 10 of the examination period. In the other sample tested (lymph node) of Patient 110-003, approximately 10% of cells exhibited viral replication and this remained steady until day 22. Patients 111-003 and 120-001 exhibited no substantial viral replication over the period tested.

Viral replication analysis was also examined in CD3+CD8+ Cytotoxic T cells from the same lesion samples in the aforementioned patients. Viral replication was observed in T cells in all patients, with replication peaking at Day 10 of the examination period in all four patients compared to days 1 and 22 (FIG. 7B).

Multiplex immunofluorescence of a sample tumor section isolated during Crossover Day 10 of a patient with unresectable anti-PD-1 refractory melanoma undergoing lerapolturev monotherapy treatment confirms viral replication (FIG. 7C-7E) in the tumor microenvironment, showing co-localization of lerapolturev (FIG. 7E) with T cells (FIG. 7C) and macrophages (FIG. 7D) in the tumor lesion section. Lerapolturev staining was predominantly observed in macrophages (FIG. 7D). These results suggest lerapolturev is infecting and replicating in a variety of immune cells in the tumor microenvironment.

### Preliminary Dose Increase Results

The new protocol amendment was implemented in January 2022 with patients beginning to receive 2.5-fold more lerapolturev per visit (1.6×10⁹ TCID₅₀) relative to the previous amendment. The protocol (LUMINOS-102) was originally designed to test lerapolturev injections in up to 6 lesions (or max dose of 6×10⁸ TCID₅₀) given every 3 to 4 weeks (Q3/4W schedule) with or without anti-PD-1 antibody therapy. Until January 2022, only one patient exhibited a clinically beneficial response.

TABLE 6
Demography and Efficacy

| | Initial | | Current | |
|---|---|---|---|---|
| | (January 2022) | | (As of September 2022) | |
| Dosing | 6.0 × 10⁸ TCID⁵⁰ | | 1.6 × 10⁹ TCID⁵⁰ | |
| Number of Patients | 14 | | 7 | |
| Age, median (range) | 63 | (41, 84) | 66 | (25, 74) |
| Sex, % male/% female | 50/50 | | 43/57 | |
| Complete Response, n (%) | 0 | | 1 | (14) |
| Disease Control Rate, n (%) | 1 | (7) | 5 | (71) |

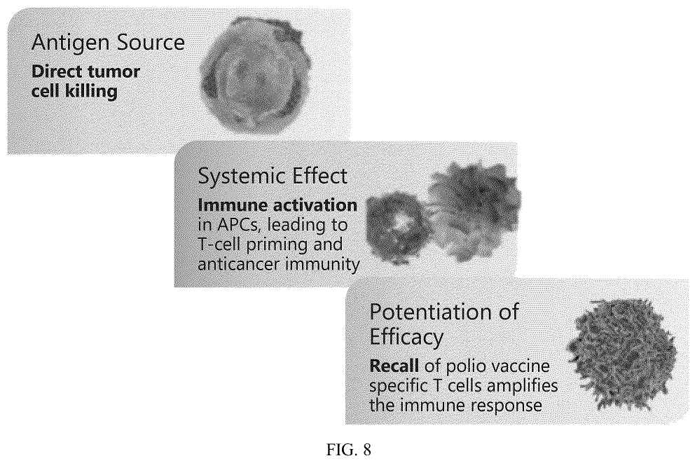

As of September 2022, 9 months after adopting the protocol amendment incorporating the dosage and frequency increase, 4 patients (71%) had exhibited a clinical beneficial response, with one patient having a complete response (14%) (Table 6). In addition, no dose-limiting toxicities (DLTs) or treatment-related serious adverse events (SAEs) were reported. These promising results suggest the increased dose and frequency of administration is increasing treatment efficacy of lerapolturev (FIG. 8).

What is claimed is:

1. A method of treating a human patient having a solid tumor, wherein the treatment comprises an induction phase and a maintenance phase;
i) the induction phase comprising one or more 7-day induction cycles, wherein lerapolturev is administered to one or more tumor lesions of the patient once per each 7-day induction cycle;
ii) the maintenance phase comprising one or more maintenance cycles, wherein lerapolturev is administered to one or more tumor lesions of the patient once per each maintenance cycle, and wherein the maintenance phase is administered following the cessation of the induction phase;
wherein lerapolturev is administered at a dose of between about 2.67×10⁸ TCID₅₀ and about 1.0×10¹⁰ TCID₅₀ at each administration.

2. The method of claim 1, wherein the maintenance cycle is selected from a 7-day cycle, a 14-day cycle, a 21-day cycle, a 28-day cycle, a 35-day cycle, or a 42-day cycle.

3. The method of claim 1, wherein the patient is PD-1/PD-L1 inhibitor intolerant.

4. The method of claim 1, further comprising administering an effective amount of an immune checkpoint inhibitor (ICI).

5. The method of claim 4, wherein the ICI is a PD-1 inhibitor.

6. The method of claim 5, wherein the PD-1 inhibitor is selected from nivolumab, pembrolizumab, pidilizumab, AMP-224, sasanlimab, spartalizumab, cemiplimab, retifanlimab, tislelizumab, camrelizumab, CS1003, or dostarlimab.

7. The method of claim 4, wherein the ICI is a PD-L1 inhibitor.

8. The method of claim 7, wherein the PD-L1 inhibitor is selected from atezolizumab, durvalumab, avelumab, envafolimab, BMS-936559, lodapolimab, cosibelimab, sugemalimab, adebrelimab, CBT-502, or BGB-A333.

9. The method of claim 1, wherein lerapolturev is administered at a dose of about 1.6×10⁹ TCID₅₀ at each administration.

10. The method of claim 1, wherein lerapolturev is administered at a dose of greater than about 1.6×10⁹ TCID₅₀ at each administration.

11. The method of claim 1, wherein the 7-day induction cycle is repeated between 2-10 times.

12. The method of claim 1, wherein the 7-day induction cycle is repeated 7 times.

13. The method of claim 1, wherein the maintenance cycle is repeated between 2-10 times.

14. The method of claim 1, wherein lerapolturev is administered to between 2-10 tumor lesions at each administration.

15. The method of claim 1, wherein the solid tumor is selected from glioblastoma multiforme (GBM), astrocytoma, oligodendroglioma, astro-oligodendroglioma, renal cell carcinoma, prostate cancer, bladder cancer, esophageal cancer, stomach cancer, pancreas cancer, colorectal cancer, liver cancer, gall bladder cancer, breast cancer, medulloblastoma, lung cancer, head and neck squamous cell carcinoma (HNSCC), melanoma, ovarian cancer, or sarcoma.

16. The method of claim 16, wherein the solid tumor is a melanoma.

17. The method of claim 17, wherein the melanoma is selected from a BRAF-mutant melanoma, a NRAS-mutant melanoma, a MEK-mutant melanoma, a KIT-mutant melanoma, a GNAQ-mutant melanoma, or a GNA11-mutant melanoma.

18. The method of claim 15, wherein the solid tumor is GBM.

19. A method of treating a human patient having a solid tumor, wherein the treatment comprises an induction phase and a maintenance phase;
i) the induction phase comprising one or more 7-day induction cycles, wherein:
(a) a chimeric poliovirus is administered to one or more tumor lesions of the patient once per each 7-day induction cycle; and
(b) an effective amount of an immune checkpoint inhibitor is administered once every three cycles or once every six cycles;
ii) the maintenance phase comprising one or more maintenance cycles, wherein:
(a) the chimeric poliovirus is administered to one or more tumor lesions of the patient once per each maintenance cycle; and
(b) an effective amount of an immune checkpoint inhibitor is administered to the patient;
wherein the maintenance cycle is selected from a 7-day cycle, a 14-day cycle, a 21-day cycle, a 28-day cycle, a 35-day cycle, or a 42-day cycle, and wherein the maintenance phase is administered following the cessation of the induction phase;
wherein the chimeric poliovirus is administered at a dose of between about 2.67×10⁸ TCID₅₀ and about 1.0×10¹⁰ TCID₅₀ at each administration.

20. A method of treating a human patient having a solid tumor, wherein the treatment comprises an induction phase and a maintenance phase;
i) the induction phase comprising one or more 7-day induction cycles, wherein:
(a) a chimeric poliovirus is administered to one or more tumor lesions of the patient once per each 7-day induction cycle; and
(b) an effective amount of an immune checkpoint inhibitor is administered once every two cycles or once every four cycles;
ii) the maintenance phase comprising one or more maintenance cycles, wherein:
(a) the chimeric poliovirus is administered to one or more tumor lesions of the patient once per each maintenance cycle; and
(b) an effective amount of an immune checkpoint inhibitor is administered to the patient;
wherein the maintenance cycle is selected from a 7-day cycle, a 14-day cycle, a 21-day cycle, a 28-day cycle, a 35-day cycle, or a 42-day cycle, and wherein the maintenance phase is administered following the cessation of the induction phase;
wherein the chimeric poliovirus is administered at a dose of between about 2.67×10⁸ TCID₅₀ and about 1.0×10¹⁰ TCID₅₀ at each administration.


SEQUENCE LISTING (3 sequences)

SEQ ID NO: 1 - DNA, 7336 bp, synthetic construct
  source 1..7336: mol_type = other DNA
ttaaaacagctctggggttgtacccaccccagaggcccacgtggcggctagtactccggtattgcggtacccttgtacgcctgttttatactcccttcccgtaacttaggaattcaacttagaagtttttcacaaagaccaatagccggtaatcagccagattactgaaggtcaagcacttctgtttccccggtcaatgttgatatgctccaacagggcaaaaacaactgcgatcgttaaccgcaaagcgcctacgcaaagcttagtagcatctttgaaatcgtttggctggtcgatccgccatttcccctggtagacctggcagatgaggctagaaataccccactggcgacagtgttctagcctgcgtggctgcctgcacaccctatgggtgtgaagccaaacaatggacaaggtgtgaagagccccgtgtgctcgctttgagtcctccggcccctgaatgtggctaaccttaaccctgcagctagagcacgtaacccaatgtgtatctagtcgtaatgagcaattgcgggatgggaccaactactttgggtgtccgtgtttcactttctcctttatatttgcttatggtgacaatatatacaatatatatattggcaccatgggagctcaggtttcatcacagaaagtgggcgcacatgaaaactcaaatagagcgtatggtggttctaccattaattacaccaccattaattattatagagattcagctagtaacgcggcttcgaaacaggacttctctcaagacccttccaagttcaccgagcccatcaaggatgtcctgataaaaacatccccaatgctaaactcgccaaacatagaggcttgcgggtatagcgatagagtactgcaattaacactgggaaactccactataaccacacaggaggcggctaattcagtagtcgcttatgggcgttggcctgaatatctgagggacagcgaagccaatccagtggaccagccgacagaaccagacgtcgctgcatgcaggttttatacgctagacaccgtgtcttggacgaaagagtcgcgagggtggtggtggaagttgcctgatgcactgcgggacatgggactctttggccaaaatatgtactaccactacctaggtaggtccgggtacaccgtgcatgtacagtgtaacgcctccaaattccaccagggggcactaggggtattcgccgtaccagagatgtgtctggccggggatagcaacaccactaccatgcacaccagctatcaaaatgccaatcctggcgagaaaggaggcactttcacgggtacgttcactcctgacgacaaccagacatcacctgcccgtaggttctgcccggtggattacctctttggaaatggcacgttattggggaatgcctttgtgttcccgcaccagataataaacctacggaccaacaactgtgctacactggtactcccttacgtgaactccctctcgatagatagtatggtaaagcacaataattggggaattgcaatattaccattggccccattaaattttgctagtgagtcctccccagagattccaatcaccttgaccatagcccctatgtgctgtgagttcaatggattaagaaacattaccctgccacgcttacagggcctgccggtcatgaacacccctggtagcaatcaatatcttactgcagacaacttccagtcaccgtgtgcgctgcctgaatttgatgtgaccccacctattgacatacccggtgaagttaagaacatgatggaattggcagaaatcgacaccatgattccctttgacttaagtgcaaaaaaaaagaacaccatggaaatgtatagggttcggttaagtgacaaaccacatacagacgatcccatactctgcctgtcactctctccagcttcagatcctaggttgtcacatactatgcttggagaaatcctaaattactacacacactgggcaggatccctgaagttcacgtttctgttctgtggatccatgatggcaactggcaaactgttggtgtcatacgcgcctcctggagccgacccaccaaagaagcgtaaggaggcgatgttgggaacacatgtgatctgggacataggactgcagtcctcatgtactatggtagtgccatggattagcaacaccacgtatcggcaaaccatagatgatagtttcaccgaaggcggatacatcagcgtcttctaccaaaccagaatagtcgtccctctttcgacacccagagagatggacatccttggttttgtgtcagcgtgtaatgacttcagcgtgcgcttgatgcgagataccacacatatagagcaaaaagcgctagcacaggggttaggtcagatgcttgaaagcatgattgacaacacagtccgtgaaacggtgggggcggcaacgtctagagacgctctcccaaacactgaagccagtggaccagcacactccaaggaaattccggcactcaccgcagtggaaactggggccacaaatccactagtcccttctgatacagtgcaaaccagacatgttgtacaacataggtcaaggtcagagtctagcatagagtctttcttcgcgcggggtgcatgcgtggccattataaccgtggataactcagcttccaccaagaataaggataagctatttacagtgtggaagatcacttataaagatactgtccagttacggaggaaattggagttcttcacctattctagatttgatatggaatttacctttgtggttactgcaaatttcactgagactaacaatgggcatgccttaaatcaagtgtaccaaattatgtacgtaccaccaggcgctccagtgcccgagaaatgggacgactacacatggcaaacctcatcaaatccatcaatcttttacacctacggaacagctccagcccggatctcggtaccgtatgttggtatttcgaacgcctattcacacttttacgacggtttttccaaagtaccactgaaggaccagtcggcagcactaggtgactccctctatggtgcagcatctctaaatgacttcggtattttggctgttagagtagtcaatgatcacaacccgaccaaggtcacctccaaaatcagagtgtatctaaaacccaaacacatcagagtctggtgcccgcgtccaccgagggcagtggcgtactacggccctggagtggattacaaggatggtacgcttacacccctctccaccaaggatctgaccacatatggattcggacaccaaaacaaagcggtgtacactgcaggttacaaaatttgcaactaccatttggccactcaggaagatttgcaaaacgcagtgaacgtcatgtggaatagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagctaggtaccagtcccatatgctcattggccatggattcgcatctccaggggattgtggtggcatactcagatgtcaccacggggtgatagggatcattactgctggtggagaagggttggttgcatttacagacattagagacttgtatgcctacgaagaagaagccatggaacaaggcatcaccaattacatagagtcacttggggccgcatttggaagtggatttactcagcagattggagacaaaataacagagttgactaatatggtgaccagtaccatcactgaaaagctacttaagaacttgatcaagatcatatcctcactagttattataactaggaattatgaagacaccacaacagtgctcgctaccctggcccttcttgggtgtgatgcttcaccatggcagtggcttagaaagaaagcatgcgatgttctggagataccttatgtcaccaagcaaggtgacagttggttgaagaagtttactgaagcatgcaacgcagctaagggactggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactagagcataccattaacaactacatacagttcaagagcaaacaccgtattgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacgaactcaagcagaatttccccccccactgtggcacacagtgatgcattagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggataaatcttccagagttagatacagtattgaccagatcactacaatgattatcaatgagagaaacagaagatccaacattggcaattgtatggaggctttgttccaaggaccactccagtataaagacttgaagattgacatcaagacgagtccccctcctgaatgtatcaatgacttgctccaagcagttgactcccaggaggtgagagattactgtgagaagaagggttggatagtcaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattaggacagcaaaggtacaagggccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacaaactttgaggaggcaattttctccaagtacgtgggtaacaaaattactgaagtggatgagcacatgaaagaggcagtagaccactatgctggccagctcatgtcactagacatcaacacagaacaaatgtgcttggaggatgccatgtatggcactgatggtctagaagcacttgatttgtccaccagtgctggctacccttatgtagcaatgggaaagaagaagagagatatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtagggtgcgatccagatttgttttggagcaaaattccggtattgatggaagagaagctgtttgcctttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggttgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctatatttgaaacagtcacatgggagaatgtaacattcttgaagagattcttcagggcagacgagaaatacccatttcttattcatccagtaatgccaatgaaggaaattcatgaatcaattagatggacaaaagatcctaggaacactcaggatcacgttcgctctctgtgcctattagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactgctgtaggggtaaatttttctttaattcggagaaaaaaaaaaaaaccc

SEQ ID NO: 2 - DNA, 109 bp, synthetic construct
  source 1..109: mol_type = other DNA
ttaaaacagctctggggttgtacccaccccagaggcccacgtggcggctagtactccggtattgcggtacccttgtacgcctgttttatactcccttcccgtaacttag

SEQ ID NO: 3 - DNA, 513 bp, synthetic construct
  source 1..513: mol_type = other DNA
gaattcaacttagaagtttttcacaaagaccaatagccggtaatcagccagattactgaaggtcaagcacttctgtttccccggtcaatgttgatatgctccaacagggcaaaaacaactgcgatcgttaaccgcaaagcgcctacgcaaagcttagtagcatctttgaaatcgtttggctggtcgatccgccatttcccctggtagacctggcagatgaggctagaaataccccactggcgacagtgttctagcctgcgtggctgcctgcacaccctatgggtgtgaagccaaacaatggacaaggtgtgaagagccccgtgtgctcgctttgagtcctccggcccctgaatgtggctaaccttaaccctgcagctagagcacgtaacccaatgtgtatctagtcgtaatgagcaattgcgggatgggaccaactactttgggtgtccgtgtttcactttctcctttatatttgcttatggtgacaatatatacaatatatatattggcacc